(19) United States
(12) Patent Application Publication
(10) Pub. No.: US 2004/0053234 A1
(43) Pub. Date: Mar. 18, 2004

(54) Polynucletides that control delta-6 desaturase genes and methods for identifying compounds for modulating delta-6 desaturase

(76) Inventors: Michael David Winther, Vancouver (CA); Heidi Lynn Smith-King, Hantsport (CA); Andre Ponton, St. Hubert (CA); Roberto Justo De Antueno, Coldbrook (CA); Stephen John Allen, Coldbrook (CA)

Correspondence Address: David J Heller, Ridout & Maybee, Suite 2400, One Queen Street East, Toronto, ON, M5C 3B1, CA

(21) Appl. No.: 10/239,652

(22) PCT Filed: Mar. 26, 2001
(86) PCT No.: PCT/CA01/00398

(30) Foreign Application Priority Data
Mar. 24, 2000 (CA) 2301158

(51) Int. Cl. C12Q 1/68; C07H 21/04; C12N 9/04; A61K 38/44

(52) U.S. Cl. 435/6; 435/69.1; 435/190; 435/320.1; 435/325; 536/23.2; 424/94.4

(57) ABSTRACT

The present invention relates to polynucleotides that control desaturase genes and to drug screening assays for identifying pharmaceutially active compounds for use in the treatment of diseases involving abnormal lipid metabolism including diabetic neuropathy, by utilizing fatty acid desaturase enzymes and the genes which encode them as targets for intervention. The drug screening method identifies nucleotides, proteins, compounds and/or other pharmacological agents, which effectively modulate the activity of desaturase enzymes or regulate the level of expression of the desaturase genes.

### FIELD OF THE INVENTION

This invention relates to the identification of nucleotides, proteins, compounds and/or pharmacological agents that either inhibit or enhance the activity of fatty acid delta-6-desaturase enzymes involved in lipid metabolism and/or effectively regulate the level of expression of the delta-6-desaturase genes, and to compounds so identified.

### BACKGROUND OF THE INVENTION

The first committed step in the biosynthetic pathway for polyunsaturated fatty acids (PUFAs) is catalyzed by an enzyme known as delta-6-desaturase (D6D) which catalyzes the synthesis of GLA from LA. This occurs in the n-6 metabolic pathway. In addition, D6D also converts ALA into stearidonic acid (SDA) in the n-3 metabolic pathway. GLA is subsequently converted as a substrate to DGLA through an elongation process, which is then converted to AA through desaturation by a different desaturase enzyme known as delta-5-desaturase (D5D). AA and DGLA are essential precursors of various important eicosanoids. These PUFAs are subsequently incorporated into membrane phospholipids and used for eicosanoid biosynthesis.

The amino acid and nucleotide sequences for human delta-6-desaturase have been found, (Cho et al., 1999a, J. Biol. Chem., 274: 471-477), and the rat delta-6-desaturase characterized to some extent (Aki et al., 1999, Biochem. Biophys. Res. Commun., 255: 575-579).

It has been reported that endogenous GLA formation is impaired in a number of disease states and subsequent administration of GLA has therapeutic effect (Horrobin D. F., 1990, Rev. Contemp. Pharmacother., 1: 1-45 and Horrobin D. F., 1992, Prog. Lipid Res., 31: 163-194). The types of diseases which have been studied include the following: atopic eczema, diabetic neuropathy, mastalgia, rheumatoid arthritis, Sjogren's syndrome, gastrointestinal disorders, viral infections and postviral fatigue, endometriosis, schizophrenia, alcoholism, Alzheimer's syndrome, cardiovascular disease, renal disease, cancer and liver disease. Also, in several other human diseases (e.g. cystic fibrosis, Cohn's disease and congenital liver disease) abnormal patterns of PUFAs attributable to insufficient dietary LA or to altered metabolism such as diminished capabilities involving desaturation or chain elongation have been described (Cook H. W., 1996, Fatty Acid Desaturation and Chain Elongation in Eukaryotes: in Biochemistry of Lipids, Lipoproteins and Membranes, Vance D. E. and Vance J. E. (eds.) Elsevier, Amsterdam, pp.150).

Furthermore, severe effects observed in experimental animals and humans in the absence of dietary essential fatty acids include a dramatic decrease in weight, dermatosis and increased permeability to water, enlarged kidneys and reduced adrenal and thyroid glands, cholesterol accumulation and altered fatty acyl composition in many tissues, impaired reproduction and ultimate death (Sinclair H. M., 1984, Hum. Nutr. Clin. Nutr., 38: 245-260).

The pathophysiology of diabetic peripheral neuropathy appears to be associated with the abnormal metabolism of essential fatty acids (Julu P., 1997, in Essential Fatty Acids and Eicosanoids, pp.168-175). This abnormal or altered lipid metabolism is reflected in the lack of incorporation of n-6 fatty acids in membrane phospholipids (Coste et al., 1999, J. Nutr. Biochem., 10: 411-420). Evidence from experimental diabetes studies in animals indicates that the formation of fatty acids by the desaturation and elongation systems is impaired which may lead to an abnormal polyunsaturated fatty acid metabolism. Based on these findings, it has been proposed that if the rate-limiting step of the reaction involving the delta-6-desaturation of LA is bypassed by way of administration of the product, GLA, it may be possible to control or reduce some of the pathophysiological symptoms associated with diabetic neuropathy (Cotter M. A. and Cameron N. E., 1997, Diabetic Neuropathy, Marius Press, Carnforth, U.K., pp. 97-119). Oils containing substantial amounts of n-6 fatty acids, in particular GLA, have prevented nerve conduction velocity deficits. Indeed, GLA treatment in diabetic rats prevented nerve conduction velocity deficits probably by the repletion of a discrete pool of arachidonic acid in phospholipids which is critical for normal nerve function (Kuruvilla et al., 1998, Prostaglandins Leukot. Essent. Fatty Acids, 59: 196-202 and Coste et al., 1999, J. Nutr. Biochem., 10: 411-420). The precise mechanism by which GLA brings about these improvements has not yet been established.

In human diabetics, trends observed in results derived from several clinical trials resemble those found in animal models. For example, multicenter clinical trials have shown promising results with GLA treatment in that the administration of GLA partially normalizes nerve conduction velocity and other neurophysiological parameters, thereby reducing symptoms of diabetic neuropathy (Keen et al. 1993, Diabetes Care, 16: 8-15). Recent studies have also shown that the therapeutic effect of GLA in diabetes can be enhanced by the addition of other compounds that affect lipid oxidation (Tomlinson D. R, 1998, Diabetes and Metabolism (Paris), 24 (suppl) 3: 79-83). Data compiled from different laboratory studies suggest that GLA is the main active n-6 PUFA for treatment of diabetic neuropathy.

However, fatty acids of the n-3 family are not as effective when tested in animal models of diabetes. Furthermore, it has been shown that the administration of n-3 fatty acids in combination with GLA can actually reduce the incorporation of GLA and subsequently impair the effectiveness of GLA in its ability to reverse nerve conduction velocity deficits (Dines et al., 1993, Diabetologia, 30: 1132-1138).

U.S. Pat. Nos. 4,806,569 and 4,826,877 teach that the conversion of LA and ALA is deficient in certain disease conditions including diabetes. The deficiency has been identified as a lack of activity of the first enzyme in the pathway, being D6D. As a consequence, diabetic patients have a higher concentration of LA with a concomitant reduction in the AA concentration. These results have been confirmed and expanded upon with the present inventors' work

Eczema is a superficial inflammation of the skin, which affects both the dermis and the epidermis. The role of polyunsaturated fatty acids in the treatment of atopic eczema was initially proposed (Hansen A. E., 1933, Proc. Soc. Exp. Biol. Med. 31: 160-161) after it was discovered that patients who suffered from eczema also had elevated serum levels of LA, but reduced levels of delta-6-desaturase products, such as GLA. These findings were later supported and expanded by other studies in which eczema patients showed low levels of serum arachidonic acid (Manku et al., 1984, Br. J. Dermatol, 110: 643-680). A placebo-controlled clinical study confirmed the therapeutic usefulness of GLA supplementation in atopic eczema (Wright et al., 1982, Lancet, 2: 1120-1122). However, it has subsequently been reported that while providing oils rich in GLA produces a symptomatic improvement for atopic eczema, the treatment does not change the underlying disease state (Chapman & Hall ed., 1992, Unsaturated Fatty Acids: Nutritional and Physiological Significance, British Nutrition Foundation, London, pp. 175).

The cytotoxic effects of GLA and EPA have been shown to be selective for cancer cells without affecting normal cells in vitro (Begin et al., 1986, J. Nat. Cancer Inst., 77: 1053-1062 and Vartak et al., 1997, Br. J. Cancer, 77: 1612-1620). In addition, through elongation and desaturation steps, GLA and EPA are precursor molecules of other PUFAs of relevant importance in oncology, such as dihomo-gamma-linolenic acid (DGLA) and docosahexaenoic acid (DHA). In this regard, several studies have shown that treatment of malignant cells with EPA, GLA and/or their metabolites leads to cell cycle arrest, induction of apoptosis, inhibition of mitosis (Seegers et al., 1997, Prostaglandins Leukot. Essent. Fatty Acids, 56: 271-280 and Lai et al., 1996, Br. J. Cancer, 74: 1375-1383) and cell proliferation (Calviello et al., 1998, Int. J. Cancer, 75: 699-705), anti-metastatic mechanisms, regulation of a metastasis-suppressor gene and occluding expression (Jiang et al, 1998a, Br. J. Cancer, 77: 731-738 and Jiang et al., 1998b, Biochem. Biophys. Res. Commun., 244: 414-420), reduction of tumor-endothelium adhesion, improvement of gap junction communications of the endothelium (Jiang et al., 1997, Prostaglandins Leukot. Essent. Fatty Acids, 56: 307-316), inhibition of urokinases (du Toit et al., 1994, Prostaglandins Leukot. Essent. Fatty Acids, 51: 121-124) and induction of E-cadherin (Jiang et al., 1995a, Br. J Cancer, 71: 5043-5048), reduction of the effects of growth factors on cancer cells (Jiang et al., 1995b, Br. J. Cancer, 71: 744-752), reversion of multi-drug resistance (Weber et al., 1994, J. Nat. Cancer Inst., 86: 638-639), and increase of the cytotoxic effects of chemotherapeutic agents (Plumb et al., 1993, Br. J. Cancer, 67: 728-733 and Anderson et al., 1998, Anticancer Res., 18: 791-800). In particular, EPA has been reported to significantly inhibit the growth of human pancreatic cancer cell lines in vitro (Falconer et al., 1994, Br. J. Cancer, 69: 826-832) and down-regulate the acute-phase response in patients with pancreatic cancer cachexia (Wigmore et al., 1997, Clin. Sci., 92: 215-221). It has also been shown that following exposure to GLA or EPA, malignant cells generate much higher levels of potentially cytotoxic superoxide radicals and lipid peroxidation products (Takeda et al., 1993, Anticancer Res., 13: 193-200).

In view of the beneficial effects of GLA, and the other essential products of D6D, there is a need for drug screening methods which identify test components which will modulate fatty acid desaturase activity or the level of desaturase gene expression for their subsequent utilization in the treatment and/or prevention of certain pathological disorders associated with abnormal lipid metabolism.

### SUMMARY OF INVENTION

The present invention is directed to mammalian fatty acid desaturase enzymes and the use of their nucleic acid and amino acid sequences in expression vectors and host systems for drug screening methods. Test components identified through these methods can be used as a basis for the formulation or innovation of therapeutic drugs, or as lead compounds to design or search for other drugs.

The invention teaches (1) the isolation, cloning and identification of the control region (i.e. promoter and other regulatory elements) of both a human and a rat fatty acid desaturase gene and (2) the use of the desaturase gene control region in drug screening methods to identify test components which can effectively modulate desaturase gene expression. The present invention incorporates the knowledge that the particular genetic elements, which are responsible for controlling desaturase gene expression, can be isolated independently of the desaturase gene encoding region (i.e. amino acid coding sequences) and, therefore, be employed to assay for agents that modulate desaturase gene expression,

The invention thus provides an isolated polynucleotide segment, comprising a polynucleotide sequence which is selected from the group consisting of: (a) a sequence comprising SEQ ID NO: 1; (b) a sequence comprising SEQ ID NO: 2; (c) a sequence which is at least 80% homologous with a sequence of any of (a) to (b); (d) a sequence which is at least 90% homologous with a sequence of any of (a) to (b); (e) a sequence which is at least 95% homologous with a sequence of any of (a) to (b); (f) a sequence which is at least 98% homologous with a sequence of any of (a) to (b); (g) a sequence which is at least 99% homologous with a sequence of any of (a) to (b); and; (h) a sequence which hybridizes to any of (a) to (g) under stringent conditions.

The invention also teaches an isolated polynucleotide segment of the invention, wherein the isolated polynucleotide segment is genomic DNA. The invention teaches a vector comprising a polynucleotide segment of the invention in a suitable vector. Also provided is a host cell comprising a polynucleotide segment of the invention in a host cell which is heterogeneous to the segment.

The invention provides a method for producing a polypeptide encoded by a gene operably linked to a polynucleotide segment of the invention comprising the step of culturing the host cell of the invention under conditions sufficient for the production of the polypeptide.

The invention includes an isolated polynucleotide fragment selected from the group consisting of: (a) a sequence having at least 15 sequential bases of nucleotides of a segment of the invention; (b) a sequence having at least 30 sequential bases of nucleotides of a segment of the invention ; and (c) a sequence having at least 50 sequential bases of nucleotides of a segment of the invention.

Also taught is a vector comprising a polynucleotide segment of the invention contained in a vector which is heterogeneous to the segment. Also taught is an isolated polynucleotide segment, comprising a polynucleotide sequence which retains substantially the same biological function or activity as the polynucleotide encoded by a segment of the invention.

The invention teaches a method for identifying a compound which inhibits or promotes the activity of a polynucleotide segment of the invention, comprising the steps of: (a) selecting a control animal having the segment and a test animal having the segment; (b) treating the test animal using a compound; and, (c) determining the relative quantity of an expression product of an operably linked polynucleotide to the segment, as between the control animal and the test animal. The animals may be mammals. The mammals may be rats.

The invention further teaches a method for identifying a compound which inhibits or promotes the activity of a polynucleotide segment of the invention, comprising the steps of: (a) selecting a host cell of the invention; (b) cloning the host cell and separating the clones into a test group and a control group; (c) treating the test group using a compound; and (d) determining the relative quantity of an expression product of a polynucleotide operably linked to the polynucleotide segment, as between the test group and the control group.

The invention teaches a method for identifying a compound which inhibits or promotes the activity of a polynucleotide segment of the invention comprising the steps of: (a) selecting a test group having a host cell of the invention a part thereof or an isolated polynucleotide thereof and a control group; (b) treating the test group using a compound; and (c) determining the relative quantity of an expression product of an operably linked polynucleotide to the segment, as between the test group and the control group.

Also taught is a composition for treating a lipid metabolism disorder comprising a compound which modulates a segment according to the invention and a pharmaceutically acceptable carrier.

The invention further teaches a method for diagnosing the presence of or a predisposition for a lipid metabolic disorder in a subject by detecting a germline alteration in a segment of the invention in the subject, comprising comparing the germline sequence of a segment of the invention from a tissue sample from the subject with the germline sequence of a wild-type of the segment, wherein an alteration in the germline sequence of the subject indicates the presence of or a predisposition to the lipid metabolic disorder. The comparing may be performed by a method selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

The invention further teaches a method for identifying a compound which inhibits or promotes the activity of a polynucleotide, wherein the polynucleotide encodes a mammalian delta-6-desaturase, comprising the steps of (a) selecting a control animal having the polynucleotide and a test animal having the polynucleotide; (b) treating the test animal using a compound; and, (c) determining the relative quantity of an expression product of the polynucleotide, as between the control animal and the test animal.

The invention teaches a method for identifying a compound which inhibits or promotes the activity of a mammalian delta-6-desaturase, comprising the steps of: (a) selecting a host cell of the invention; (b) cloning the host cell and separating the clones into a test group and a control group; (c) treating the test group using a compound; and (d) determining the relative quantity of an expression product of an expression polynucleotide operably linked to the polynucleotide segment, as between the test group and the control group.

The invention teaches a method for identifying a compound which inhibits or promotes the activity of a mammalian delta-6-desaturase, comprising the steps of: (a) selecting a test group haying a host cell of the invention a part thereof or an isolated polynucleotide thereof and a control group; (b) treating the test group using a compound; and (c) determining the relative quantity or relative activity of a product of the polynucleotide segment or of the polynucleotide segment, as between the test group and the control group.

The invention teaches a method for identifying a compound which inhibits or promotes the activity of a mammalian delta-6-desaturase, comprising the steps of: (a) selecting a control animal having a polypeptide segment of the invention and a test animal having the polypeptide segment; (b) treating the test animal using a compound; (c) determining the relative quantity or relative activity of an expression product of the polypeptide segment or of the the polypeptide segment, as between the control animal and the test animal.

The relative activity of the expression product may be determined by assaying for a conversion of 18:2n6 to 22:5n6. The relative activity of the expression product may be determined by assaying for a conversion of 18:3n3 to 22:6n3. The relative activity of the expression product may be determined by assaying for a conversion of 16:0 to 22:4n9.

The invention further teaches a use of a method according to the invention for identifying a modulator that modulates lipid metabolism disorders.

The invention also teaches a composition for treating a lipid metabolism disorder comprising a compound identified by any one of the methods of the invention and a pharmaceutically acceptable carrier.

The invention teaches a method for diagnosing the presence of or a predisposition for a lipid metabolism disorder in a subject by detecting a germline alteration in a polynucleotide of the invention in the subject, wherein the polynucleotide encodes a mammalian delta-6-desaturasecomprising comparing the germline sequence of the polynucleotide from a tissue sample from the subject with the germline sequence of a wild-type of the polynucleotide, wherein an alteration in the germline sequence of the subject indicates the presence of or a predisposition to the lipid metabolism disorder. The method may be selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

The invention further teaches a method for diagnosing the presence of or a predisposition for a lipid metabolic disorder in a subject, comprising comparing the polypeptide sequence of a control region of delta-6desaturase from a tissue sample from the subject with the sequence of a wild-type of the delta-6desaturase, wherein an alteration in the sequence of the subject as compared to the wild-type indicates the presence of or a predisposition to the lipid metabolic disorder.

The invention further teaches a method for identifying a compound which inhibits or promotes the activity of control regions of mammalian delta-6- and/or delta-5-desaturases, comprising the steps of. (a) selecting one or more host cells comprising the polynucleotides, wherein such host cells are heterogeneous to the polynucleotides; (b) cloning the host cells and separating the clones into a test group and a control group; (c) treating the test group using a compound; and (d) determining the relative quantities of expression products of operably linked polynucleotides to the control regions, as between the test group and the control group. Also taught is a composition for treating a lipid metabolism disorder comprising a compound identified by a method of the invention and a pharmaceutically acceptable carrier.

The invention also teaches a compound identified by the methods of the invention. The invention further teaches the use of a compound as the inventioned in the invention for treating a lipid metabolism disorder.

A host cell of the invention may be a spheroplast. The spheroplast may be a Saccharomyces cerevisiae.

The disorders of the invention may be selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications. The diabetic complication may be selected from the group consisting of diabetic neuropathy, nephropathy and retinopathy.

The compounds of the invention may be selected from the group consisting of small organic molecules, peptides, polypeptides, antisense molecules, oligonucleotides, polynucleotides, fatty acids and derivatives thereof.

The invention further teaches the use of a composition of the invention for treating a lipid metabolic disorders.

These and other advantages and features of novelty, which characterize the invention, are pointed out with particularity in the inventions annexed hereto and forming a part hereof. For a better understanding of the invention, its advantages, and objects obtained by its use, reference may be made to the accompanying drawings and descriptive matter, in which there is illustrated and described preferred embodiments of the invention.

### BRIEF DESCRIPTION OF THE DRAWINGS

In the following description, the invention will be explained in detail with the aid of the accompanying figures, which illustrate preferred embodiments of the present invention and in which:

FIG. 1 shows the nucleic acid sequence (SEQ ID NO:1) of the rD6D-1 control region;

FIG. 2 shows the nucleic acid sequence (SEQ ID NO:2) of the hD6D-1 control region;

FIG. 3 shows the nucleic acid sequence of the rD6D-1 coding portion of the fatty acid desaturase gene;

FIG. 4 shows the nucleic acid sequence of the hD6D-1 coding portion of the fatty acid desaturase gene;

FIG. 5 shows the amino acid sequences of the native rD6D-1 and C-terminal tagged enzymes, respectively;

FIG. 6 shows the amino acid sequences of the native hD6D-1 and C-terminal tagged enzymes, respectively,

FIG. 7 shows the relative locations of the human “desaturase 1” (hD5D), “desaturase 2” (hD6D-2) and “desaturase 3” (hD6D1) genes on a segment of DNA from chromosome 11;

FIG. 8 shows alternative splicing sites for exon 1 of the hD5D gene. “CRE” means cAMP responsive element.

FIG. 9 shows the genomic exon-intron organization of hD5D, hD6D-2 and hD6D-1;

FIG. 10 illustrates a transmembrane hidden Markov model prediction for the hD5D gene;

FIG. 11 shows the multiple alignments for fatty acid desaturases of different organisms highlighting the cytochrome b₅ motif and conserved histidine boxes. Identical or highly conserved residues are shaded;

FIG. 12 is a dendogram showing the similarities or relatedness of the three human fatty acid desaturases to fatty acid desaturases from other organisms;

FIG. 13 is a schematic representation of plasmid pYr5003.1 (7104 bp). The rat delta-6-desaturase-1 coding sequence is shown between restriction sites for XbaI and HindIII;

FIG. 14 is a schematic representation of plasmid pTr5404.1 (7207 bp) that contains the N-terminal tags. The rat delta-6-desaturase 1 coding sequence is shown between restriction sites for XbaI and HindIII;

FIG. 15 is a schematic representation of plasmid pYh5001.2 (7116 bp). The human delta-6-desaturase 1 coding sequence is shown between restriction sites for XbaI and HindIII;

FIG. 16 is a schematic representation of plasmid pTh5002.1 (7207 bp), which contains the N-terminal tags. The human delta-6-desaturase 1 coding sequence is shown between restriction sites for XbaI and HindIII;

FIG. 17 is a schematic representation of plasmid pRr4001.1. The rat delta-6-desaturase 1 control region is shown between restriction sites for XhoI and SacI;

FIG. 18 is a schematic representation of plasmid pRh4002.1. The human delta-6-desaturase 1 control region is shown between the two restriction sites for KpnI;

FIG. 19 is a schematic representation of plasmid pGh4015.1 . The human delta-6-desaturase 1 control region is shown between restriction sites for XhoI and KpnI;

FIG. 20 illustrates the expression of the CAT reporter gene under the control of the rat (pRr4001.1) or the human (pRh4002.1) delta-6-desaturase 1 gene control region as compared to its expression from the SV40 promoter (pCAT-3-CTL) after transfection in ZR-75-1 cells. The levels of expression were determined by the CAT enzymatic activity and expressed relative to the pCAT-3-CTL. The empty vector (pCAT-3-Enhancer) was also transfected as a negative control. Bars indicate standard deviation from three experiments;

FIG. 21 shows a High Performance Liquid Chromatographic (HPLC) analysis of radiolabelled methyl esters of fatty acids from yeast transformed with pYES2 (panel A) or pYr5003.1 (panel B) incubated with linoleic acid, [1-¹⁴C]-18:2n-6;

FIG. 22 shows a gas chromatographic analysis of methyl esters of fatty acids from yeast transformed with pYES2 (panel A) or pYr5003.1 (panel B) incubated with linoleic acid, 18:2n-6. The arrow indicates the presence of a new fatty acid, gamma-linolenic acid, 18:3n-6. The common peaks to both yeast were identified as: a, 14:0; b, 16:0; c, 16:1n-7; d, 18:0; f, 18:1n-9;

FIG. 23 shows the percent conversion of alpha-linolenic acid (18:3n-3) into 18:4n-3 in Saccharomyces cerevisiae cells transformed with pYr5003.1 at different induction time points with galactose;

FIG. 24 illustrates the percentage of radioactivity from [1-¹⁴C]-18:3n-3 recovered in spheroplasts and yeast whole cells transformed with pYr5003.1;

FIG. 25 illustrates the percentage of radioactivity from [1-¹⁴C]-24:4n-6 recovered in spheroplasts and yeast whole cells transformed with pYr5003.1; and

FIG. 26 illustrates the percentage of delta-6-desaturation of [1-¹⁴C]-18:3n-3 in spheroplasts and yeast whole cells transformed with pYr5003.1.

### DETAILED DESCRIPTION OF THE INVENTION

The present invention has evolved from observations that oral supplementation of naturally occurring fatty acids has had some therapeutic benefit in counteracting existing metabolic deficiencies prevalent in certain disease conditions. Using this observation, nutritional and pharmaceutical products have hitherto been developed using oils rich in selected fatty acids.

However, to address new strategies for therapeutic intervention, it is necessary to go beyond the measurement of lipid levels and lipid supplementation and directly measure actual enzyme activities and the regulation of expression of the genes from which these enzymes are encoded. The human genes that are uniquely involved and responsible for expressing the various enzymes utilized along these pathways have hitherto been mostly uncharacterized.

In this regard, the development of an experimental model that can be manipulated to study the expression of genetic material isolated from humans and other species is beneficial in establishing the role and function which these genes and their encoded proteins exhibit in PUFA metabolism. This is particularly so in recognition of the fact that the relationship between a protein's unique role in a metabolic pathway and the expression of the gene encoding that protein is normally a well coordinated event such that subtle deviations can lead to abnormal physiological processes. Moreover, such a system would facilitate the discovery and identification of candidate drug targets effective in correcting abnormalities or imbalances in lipid metabolic changes associated with certain pathological conditions, such as diabetic neuropathy.

The invention teaches (1) the isolation, cloning and identification of the control region (i.e. promoter and other regulatory elements) of both a human and a rat fatty acid desaturase gene and (2) the use of the desaturase gene control region in drug screening methods to identify test components which can effectively modulate desaturase gene expression. The present invention incorporates the knowledge that the particular genetic elements, which are responsible for controlling desaturase gene expression, can be isolated independently of the desaturase gene encoding region (i.e. amino acid coding sequences) and, therefore, be employed to assay for agents that modulate desaturase gene expression.

The utility of such genetic control and regulatory elements ranges from their use as tissue specific promoters that drive gene expression to the fine-tuning of metabolic processes involved in biochemical pathways. Accordingly, cloning of the control regions of the desaturase genes provides a powerful tool for dissecting the role of desaturase gene expression and inducing modifications thereof, which can eliminate or control alterations associated with metabolic disorders. Therefore, the identification and characterization of the control regions of desaturase genes allow us to identify and understand the role of discrete regulatory elements located in desaturase control regions as well as to discover potential pharmacological modulators of desaturase gene expression.

Another object of the invention is to provide methods that are designed to screen for nucleotides, proteins, compounds or pharmacological agents that regulate the level of expression of the genes that encode fatty acid desaturase enzymes, i.e. various components that act as enhancers or inhibitors of desaturase gene expression and hence, modify the desaturase enzyme concentration in tissues. To this end, cell-based and/or cell lysate assays are used to detect components that modulate the transcriptional activity of the desaturase genes. Such experimental methods make it possible to screen large collections of natural or synthetic compounds for therapeutic agents that affect desaturase gene expression.

Therefore, an object of the present invention is to provide methods for the screening of nucleotides, proteins, compounds or pharmacological agents that modulate fatty acid desaturase enzyme activity, i.e. various components that act as enhancers or inhibitors of desaturation and hence, modify unsaturated fatty acid biosynthesis. To this end, cell-based and/or cell lysate assays are used to detect components that modulate the activity of the desaturase enzymes. Such experimental methods make it possible to screen large collections of natural or synthetic compounds for therapeutic agents that affect desaturase enzyme activity.

For example, when an unsaturated fatty acid metabolite is to be produced in vivo, the substrate for the corresponding fatty acid desaturase will normally already be present. In the case where the enzymatic activity of a native fatty acid desaturase is altered, the administration of an appropriate therapeutic agent can remedy this alteration through its direct action on the enzyme. As a result, the native desaturase can ultimately act on its substrate, already present in the cell, and in vivo synthesis of the required fatty acid product is achieved. Accordingly, desaturase activity can either be restored or increased in conditions where such activity essential to fatty acid biosynthesis is abnormal.

Similarly, in the case where expression of a native desaturase gene is reduced, the administration of an appropriate therapeutic agent can remedy this effect through its direct action on functional or regulatory elements within the control region of the desaturase gene. As a result, increased expression of the gene takes place and hence, in vivo synthesis of the required desaturase enzyme is restored or increased in conditions where such activity essential to fatty acid biosynthesis is abnormal.

Therefore, isolated nucleic acid sequences encoding these desaturase enzymes have utility in constructing in vivo and/or in vitro experimental models for identifying test components which modulate mammalian fatty acid desaturase activity and/or the level and regulation of desaturase gene expression. Furthermore, the modulation or regulation of fatty acid desaturase enzyme activity or gene expression by various test components will be identified by the methods disclosed herein and hence, be used to reduce disease processes or symptoms.

It is understood that the present invention is not limited to the particular methodology, protocols, cell lines, vectors, and reagents described herein. Generally, the laboratory procedures in cell culture and molecular genetics described below are those well known and commonly employed in the art. Standard techniques are used for recombinant nucleic acid methods, polynucleotide synthesis, microbial culture, transformation, transfection, etc. Generally, enzymatic reactions and purification steps are performed according to the manufacturer's specifications. Although any methods and materials similar or equivalent to those described herein can be used in the practice or testing of the present invention, the preferred methods, devices, and materials are described below.

Definitions

The following illustrative explanations are provided to facilitate understanding of certain terms used frequently herein. The explanations are provided as a convenience and are not limitative of the invention.

Agonist refers to any molecule or pharmaceutical agent, such as a drug or hormone, which enhances the activity of another molecule.

Antagonist refers to any molecule or pharmaceutical agent, such as a drug or hormone, which inhibits or extinguishes the activity of another molecule.

Chemical Derivative. As used herein, a molecule is the to be a “chemical derivative” of another molecule when it contains additional chemical moieties not normally a part of the molecule. Such moieties can improve the molecule's solubility, absorption, biological half-life, and the like. The moieties can alternatively decrease the toxicity of the molecule, eliminate or attenuate any undesirable side effect of the molecule, and the like. Moieties capable of mediating such effects are disclosed in Mack E. W., 1990, Remington's Pharmaceutical Sciences, Mack Publishing Company, Easton, Pa., 13^{th} edition. Procedures for coupling such moieties to a molecule are well known in the art.

C mpositions include genes, proteins, polynucleotides, peptides, compounds, drugs, and pharmacological agents.

Control region refers to a nucleic acid sequence capable of; or required for, assisting or impeding initiation, termination, or otherwise regulating the transcription of a gene. The control region may include a promoter, enhancer, silencer and/or any other regulatory element A control region also includes a nucleic acid sequence that may or may not be independently or exclusively sufficient to initiate, terminate, or otherwise regulate transcription, however, is capable of effecting such regulation in association with other nucleic acid sequences.

Delta-5-Desaturase (D5D) is an enzyme that is capable of introducing a double bond between carbons 5 and 6 from the carboxyl group in a fatty acid molecule.

Delta-6-Desaturase (D6D) is an enzyme which is capable of introducing a double bond between carbons 6 and 7 from the carboxyl group in a fatty acid molecule.

Desaturase refers to a fatty acid desaturase, which is an enzyme capable of generating a double bond in the hydrocarbon region of a fatty acid molecule.

Disorder as used herein refers to derangement or abnormality of structure or function. Disorder includes disease.

Drug. Drugs include, but are not limited to proteins, peptides, degenerate peptides, agents purified from conditioned cell medium, organic molecules, inorganic molecules, antibodies or oligonucleotides. The drug can be naturally occurring or synthetically or recombinantly produced.

Enhancer is a nucleic acid sequence comprising a DNA regulatory element that enhances or increases transcription when bound by a specific transcription factor or factors. Moreover, an enhancer may function in either orientation and in any location (upstream or downstream relative to the promoter) to effect and generate increased levels of gene expression when bound by specific factors. In addition, according to the present invention, an enhancer also refers to a compound (i.e. test compound) that increases or promotes the enzymatic activity of the fatty acid regulated gene, and/or increases or promotes the transcription of the gene.

Fatty Acids are a class of compounds comprising a long saturated or mono or polyunsaturated hydrocarbon chain and a terminal carboxyl group.

Functional Derivative. A “functional derivative” of a sequence, either protein or nucleic acid, is a molecule that possesses a biological activity (either functional or structural) that is substantially similar to a biological activity of the protein or nucleic acid sequence. A functional derivative of a protein can contain post-translational modifications such as covalently linked carbohydrate, depending on the necessity of such modifications for the performance of a specific function. The term “functional derivative” is intended to include the “fragments,” “segments,” “variants,” “analogs,” or “chemical derivatives” of a molecule.

Gene refers to a nucleic acid molecule or a portion thereof, the sequence of which includes information required for the production of a particular protein or polypeptide chain. A full-length sequence or any portion of the coding sequence can encode the polypeptide, so long as the functional activity of the protein is retained. A gene may comprise regions preceding and following the coding region as well as intervening sequences (introns) between individual coding segments (exons). A “heterologous” region of a nucleic acid construct (i.e. a heterologous gene) is an identifiable segment of DNA within a larger nucleic acid construct that is not found in association with the other genetic components of the construct in nature. Thus, when the heterologous gene encodes a mammalian fatty acid regulated gene, a promoter that does not flank the structural genomic DNA in the genome of the source organism will usually flank the gene.

Host system may comprise a cell, tissue, organ, organism or any part thereof, which provides an environment or conditions that allow for, or enable, transcription and/or transcription.

Identity, similarity, homology or homologous, refer to relationships between two or more polynucleotide sequences, as determined by comparing the sequences. In the art, identity also means the degree of sequence relatedness between polynucleotide sequences, as the case may be, as determined by the match between strings of such sequences. Both identity and similarity can be readily calculated (Lesk A.M., ed., 1988, Computational Molecular Biology, Oxford University Press, NY; Smith D. W., ed., 1993, Biocomputing: Informatics and Genome Project, Academic Press, NY; Griffin A. M. and Griffin H. G., eds., 1994, Computer Analysis of Sequence Data, Part 1, Humana Press, NJ; von Heijne G., 1987, Sequence Analysis in Molecular Biology, Academic Press, NY and Gribskov M. and Devereux J., eds., 1991, Sequence Analysis Primer, M Stockton Press, NY). While there exist a number of methods to measure identity and similarity between two polynucleotide sequences, both terms are well known to skilled artisans (von Heijne G., 1987, Sequence Analysis in Molecular Biology, Academic Press, NY; Gribskov M. and Devereux J., eds., 1991, Sequence Analysis Primer, M Stockton Press, NY; Carillo H. and Lipman D., 1988, SIAM J. Applied Math., 48: 1073). Methods commonly employed to determine identity or similarity between sequences include, but are not limited to those disclosed in Carillo H. and Lipman D., 1988, SIAM J. Applied Math., 48: 1073. Methods to determine identity and similarity are codified in computer programs. Computer program methods to determine identity and similarity between two sequences include, but are not limited to, GCG program package (Devereux et al., 1984, Nucl. Acid Res., 12: 387-395), BLASTP, BLASTN and FASTA (Altschul et al., 1990, J. Molec. Biol, 215: 403-410).

Isolated means altered “by the hand of man” from its natural state; i.e., that, if it occurs in nature, it has been changed or removed from its original environment, or both. For example, a naturally occurring polynucleotide naturally present in a living organism in its natural state is not “isolated,” but the same polynucleotide separated from coexisting materials of its natural state is “isolated”, as the term is employed herein. As part of or following isolation, such polynucleotides can be joined to other polynucleotides, such as DNA, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNA still would be isolated, as the term is used herein, because they would not be in their naturally occurring form or environment. Similarly, the polynucleotides may occur in a composition, such as a media formulations, solutions for introduction of polynucleotides, for example, into cells, compositions or solutions for chemical or enzymatic reactions, for instance, which are not naturally occurring compositions, and, therein remain isolated polynucleotides within the meaning of that term as it is employed herein.

Mutation. A “mutation” is any detectable change in the genetic material. A mutation can be any (or a combination of) detectable, unnatural change affecting the chemical or physical constitution, mutability, replication, phenotypic function, or recombination of one or more deoxyribonucleotides; nucleotides can be added, deleted, substituted for, inverted, or transposed to new positions with and without inversion. Mutations can occur spontaneously and can be induced experimentally by application of mutagens or by site directed mutagenesis. A mutant polypeptide can result from a mutant nucleic acid molecule.

Nucleic acid construct refers to any genetic element, including, but not limited to, plasmids and vectors, that incorporate polynucleotide sequences. For example, a nucleic acid construct may be a vector comprising a promoter or control region that is operably linked to a heterologous gene.

Operably linked as used herein indicates the association of a promoter or control region of a nucleic acid construct with a heterologous gene such that the presence or modulation of the promoter or control region influences the transcription of the heterologous gene, including genes for reporter sequences. Operably linked sequences may also include two segments that are transcribed onto the same RNA transcript. Thus, two sequences, such as a promoter and a “reporter sequence” are operably linked if transcription commencing in the promoter produces an RNA transcript of the reporter sequence.

Plasmids. Starting plasmids disclosed herein are either commercially available, publicly available, or can be constructed from available plasmids by routine application of well-known, published procedures. Many plasmids and other cloning and expression vectors that can be used in accordance with the present invention are well known and readily available to those of skill in the art. Moreover, those of skill readily may construct any number of other plasmids suitable for use in the invention.

Polynucleotides(s) of the present invention may be in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded polynucleotides may be the coding strand, also known as the sense strand, or it may be the non-coding strand, also referred to as the anti-sense strand. Polynucleotides generally refer to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded, or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term polynucleotide also includes DNA or DNA that contain one or more modified bases. Thus, DNA or DNA with backbones modified for stability or for other reasons are “polynucleotides” as that term is intended herein. Moreover, DNA or DNA comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. Polynucleotides embraces short polynucleotides often referred to as oligonucleotide(s). It will also be appreciated that RNA made by transcription of this doubled stranded nucleotide sequence, and an antisense strand of a nucleic acid molecule of the invention or an oligonucleotide fragment of the nucleic acid molecule, are contemplated within the scope of the invention. An antisense sequence is constructed by inverting the sequence of a nucleic acid molecule of the invention, relative to its normal presentation for transcription. Preferably, an antisense sequence is constructed by inverting a region preceding the initiation codon or an unconserved region. The antisense sequences may be constructed using chemical synthesis and enzymatic ligation reactions using procedures known in the art.

Promoter refers to a nucleic acid sequence comprising a DNA regulatory element capable of binding RNA polymerase directly or indirectly to initiate transcription of a downstream (3′ direction) gene. In accordance with the present invention, a promoter of a nucleic acid construct that includes a nucleotide sequence, wherein the nucleotide sequence may be linked to a heterologous gene such that the induction of the promoter influences the transcription of the heterologous gene.

Purified. A “purified” protein or nucleic acid is a protein or nucleic acid preparation that is generally free of contaminants, whether produced recombinantly, chemically synthesized or purified from a natural source.

Recombinant refers to recombined or new combinations of nucleic acid sequences, genes, or fragments thereof which are produced by recombinant DNA techniques and are distinct from a naturally occurring nucleic acid sequence

Regulatory element refers to a deoxyribonucleotide sequence comprising the whole, or a portion of, a nucleic acid sequence to which an activated transcriptional regulatory protein, or a complex comprising one or more activated transcriptional regulatory proteins, binds so as to transcriptionally modulate the expression of an associated gene or genes, including heterologous genes.

Reporter gene is a nucleic acid coding sequence whose product is a polypeptide or protein that, is not otherwise produced by the host cell or host system, or which is produced in minimal or negligible amounts in the host cell or host system, and which is detectable by various known methods such that the reporter gene product may be quantitatively assayed to analyze the level of transcriptional activity in a host cell or host system. Examples include genes for luciferase, chloramphenicol acetyl transferase (CAT), beta-galactosidase, secreted placental alkaline phosphatase and other secreted enzymes.

Silencer refers to a nucleic acid sequence or segment of a DNA control region such that the presence of the silencer sequence in the region of a target gene suppresses the transcription of the target gene at the promoter through its actions as a discrete DNA segment or through the actions of trans-acting factors that bind to these genetic elements and consequently effect a negative control on the expression of a target gene.

Stringent hybridization conditions are those which are stringent enough to provide specificity, reduce the number of mismatches and yet are sufficiently flexible to allow formation of stable hybrids at an acceptable rate. Such conditions are known to those skilled in the art and are described, for example, in Sambrook et al., 1989, Molecular Cloning, 2nd Edition, Cold Spring Harbor Laboratory Press, Cold Spring Harbour, NY or Ausubel et al., 1994-, Current Protocols in Molecular Biology, John Wiley & Sons, NY. By way of example only, stringent hybridization with short nucleotides may be carried out at 5-10° C. below the T_{M} using high concentrations of probe such as 0.01-1.0 pmole/ml. Preferably, the term “stringent conditions” means hybridization will occur only if there is at least 95% and preferably at least 97% identity between the sequences.

Tag refers to a specific short amino acid sequence, or the oligonucleotide sequence that encodes it, wherein the amino acid or nucleic acid sequence may comprise or encode, for example, a c-myc epitope and/or a string of six histidine residues recognizable by commercially available antibodies. In practice, a tag facilitates the subsequent identification and purification of a tagged protein.

Tagged protein as used herein refers to a protein comprising a linked tag sequence. For example, a tagged protein includes a mammalian fatty acid regulated polypeptide linked to a c-myc epitope and six histidine residues at the carboxyl terminus of the amino acid sequence.

Test compounds as used herein encompass small molecules (e.g. small organic molecules), pharmacological compounds or agents, peptides, proteins, antibodies or antibody fragments, and nucleic acid sequences, including DNA and RNA sequences.

Transfection refers to a process whereby exogenous or heterologous DNA (i.e. a nucleic acid construct) is introduced into a recipient eukaryotic host cell. Therefore, in eukaryotic cells, the acquisition of exogenous DNA into a host cell is referred to as transfection. In prokaryotes and eukaryotes (for example, yeast and mammalian cells) introduced DNA may be maintained on an episomal element such as a plasmid or integrated into the host genome. With respect to eukaryotic cells, a stably transfected cell is one in which the introduced DNA has become integrated into a chromosome so that it is inherited by daughter cells through chromosome replication. This stability is demonstrated by the ability of the eukaryotic cell to establish cell lines or clones comprised of a population of daughter cells containing the introduced DNA.

Transformation refers to a process whereby exogenous or heterologous DNA (i.e. a nucleic acid construct) is introduced into a recipient prokaryotic host cell. Therefore, in prokaryotic cells, the acquisition of exogenous DNA into a host cell is referred to as transformation. Transformation in eukaryotes refers to the conversion or transformation of eukaryotic cells to a state of unrestrained growth in culture, resembling a tumorigenic condition. In prokaryotes and eukaryotes (for example, yeast and mammalian cells) introduced DNA may be maintained on an episomal element such as a plasmid or integrated into the host genome. With prokaryotic cells, a stably transformed bacterial cell is one in which the introduced DNA has become integrated into a chromosome so that it is inherited by daughter cells through chromosome replication. This stability is demonstrated by the ability of the prokaryotic cell to establish cell lines or clones comprised of a population of daughter cells containing the introduced DNA.

Transfection/transformation as used herein refers to a process whereby exogenous or heterologous DNA (e.g. a nucleic acid construct) has been introduced into a eukaryotic or prokaryotic host cell or into a host system.

Variant(s) of polynucleotides are polynucleotides that differ in nucleotide sequence from another, reference polynucleotide. A “variant” of a protein or nucleic acid is meant to refer to a molecule substantially similar in structure and biological activity to either the protein or nucleic acid. Thus, provided that two molecules possess a common activity and can substitute for each other, they are considered variants as that term is used herein even if the composition or secondary, tertiary, or quaternary structure of one of the molecules is not identical to that found in the other, or if the amino acid or nucleotide sequence is not identical. Generally, differences are limited so that the nucleotide sequences of the reference and the variant are closely similar overall and, in many regions, identical. Changes in the nucleotide sequence of the variant may be silent. That is, they may not alter the amino acids encoded by the polynucleotide. Where alterations are limited to silent changes of this type a variant will encode a polypeptide or polynucleotide with the same amino acid sequence as the reference. Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide or polynucleotide encoded by the reference sequence.

Vector is a plasmid or phage DNA or other DNA sequence into which DNA can be inserted to be cloned. The vector can replicate autonomously in a host cell, and can be further characterized by one or a small number of endonuclease recognition sites at which such DNA sequences can be cut in a determinable fashion and into which DNA can be inserted. The vector can further contain a marker suitable for use in the identification of cells transformed with the vector. Markers, for example, are tetracycline resistance or ampicillin resistance. The words “cloning vehicle” are sometimes used for “vector.”

It will be appreciated that the invention includes nucleotide or amino acid sequences which have substantial sequence homology with, respectively, the subject polynucleotides or polypeptides. The term “sequences having substantial sequence homology” means those nucleotide and amino acid sequences which have slight or inconsequential sequence variations from the subject polynucleotides; i.e. the homologous sequences function in substantially the same manner to produce substantially the same polypeptides as the actual sequences. The variations may be attributable to local mutations or structural modifications. It is expected that a sequence having 85-90% sequence homology with the DNA sequence of the invention will provide functional subject polypeptides which retain substantially the same biological function or activity as the polynucleotide encoded by the subject polynucleotides. Further embodiments of the invention are polynucleotides that are at least 70% identical over their entire length to a subject polynucleotide, and polynucleotides which are complementary to such polynucleotides. Other embodiments are polynucleotides that comprise a region that is at least 80% identical over their entire length to a subject polynucleotide and polynucleotides complementary thereto. This includes polynucleotides at least 90% identical over their entire length to the same, and among these embodiments are polynucleotides with at least 95% homology. Furthermore, those with at least 97% are highly preferred among those with at least 95%, and among these those with at least 98% and at least 99% are particularly highly preferred, with at least 99% being the more preferred.

The present invention encompasses the use of individual coding (e.g. open reading frame) and non-coding portions (e.g. control region) of mammalian desaturase genes, preferably human and rat desaturase genes, in recombinant DNA constructs to enable their expression/operability in host systems for drug screening purposes.

In accordance with the present invention, nucleic acid sequences which encode fatty acid desaturases, fragments of the nucleic acid sequences, tagged protein sequences or functional equivalents thereof may be used in recombinant DNA constructs that direct the expression of desaturases in appropriate host systems. Due to the inherent degeneracy of the genetic code, other DNA sequences, which encode substantially the same or a functionally equivalent amino acid sequence, may be used to clone and express mammalian fatty acid desaturases.

Furthermore, the nucleic acid sequences of the present invention can be engineered in order to alter a desaturase coding or control sequence for a variety of reasons, including but not limited to, alterations which modify the cloning, processing and/or expression of the gene product. For example, mutations may be introduced using techniques, which are well known in the art, e.g. site-directed mutagenesis to introduce endonuclease recognition sites, to alter glycosylation patterns, to change codon preference, etc.

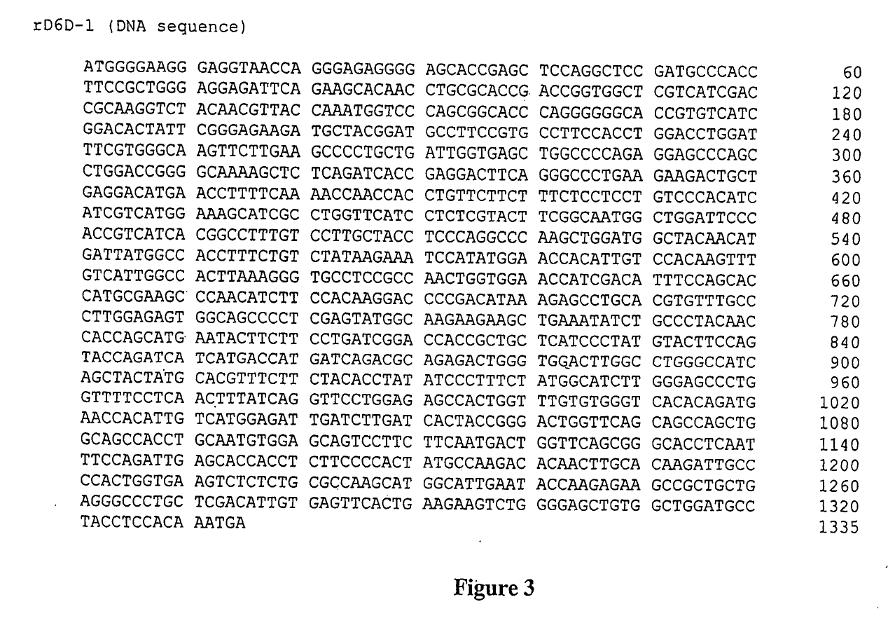

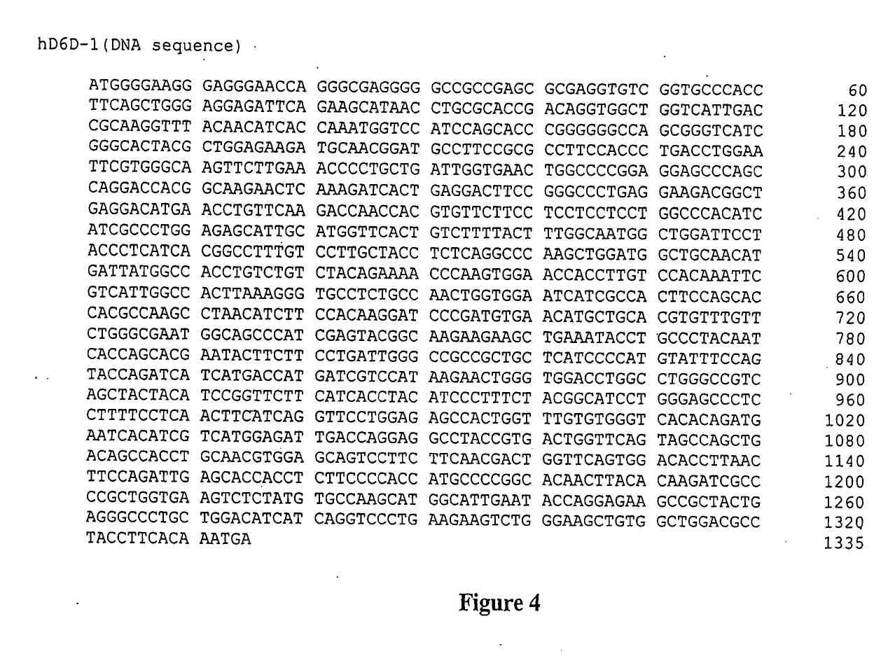

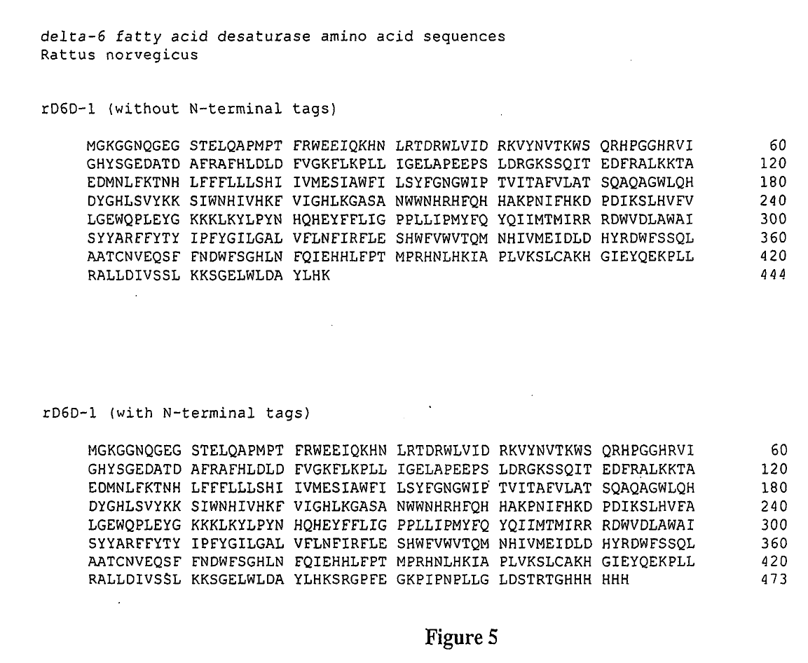

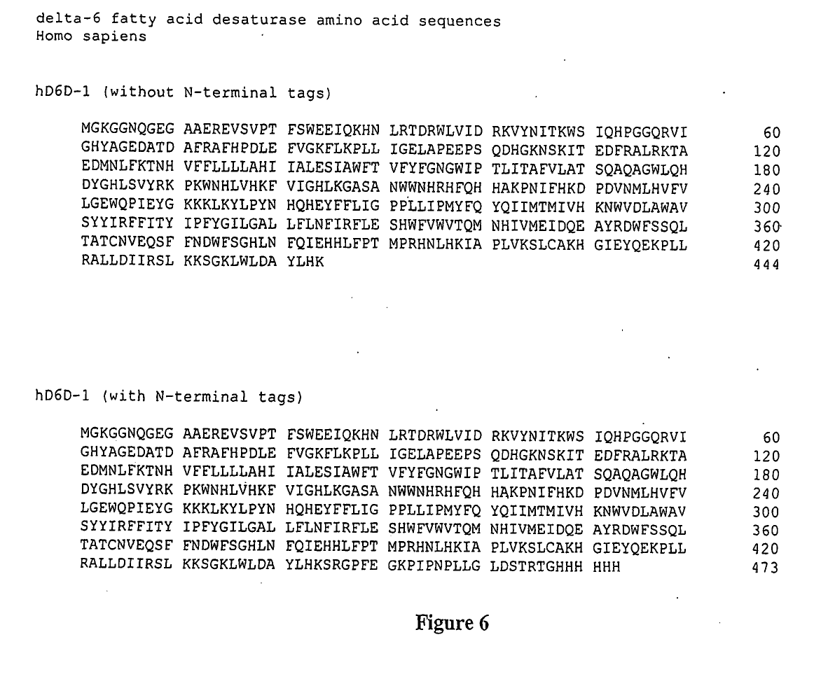

In a particular embodiment, the invention encompasses polynucleotides encoding a functional rat delta-6-desaturase (rD6D-1) and a human delta-6-desaturase (hD6D-l) having the nucleic acid sequences illustrated in FIGS. 3 and 4, respectively. The deduced amino acid sequences encoded by the nucleic acid sequences of rD6D-1 and hD6D-1 are illustrated in FIG. 5 and FIG. 6 respectively.

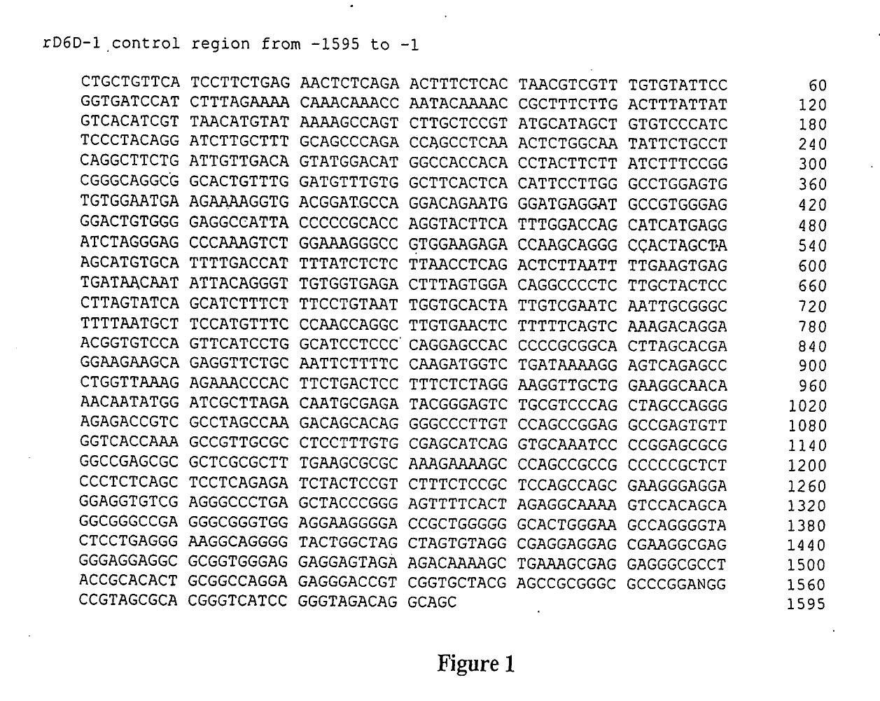

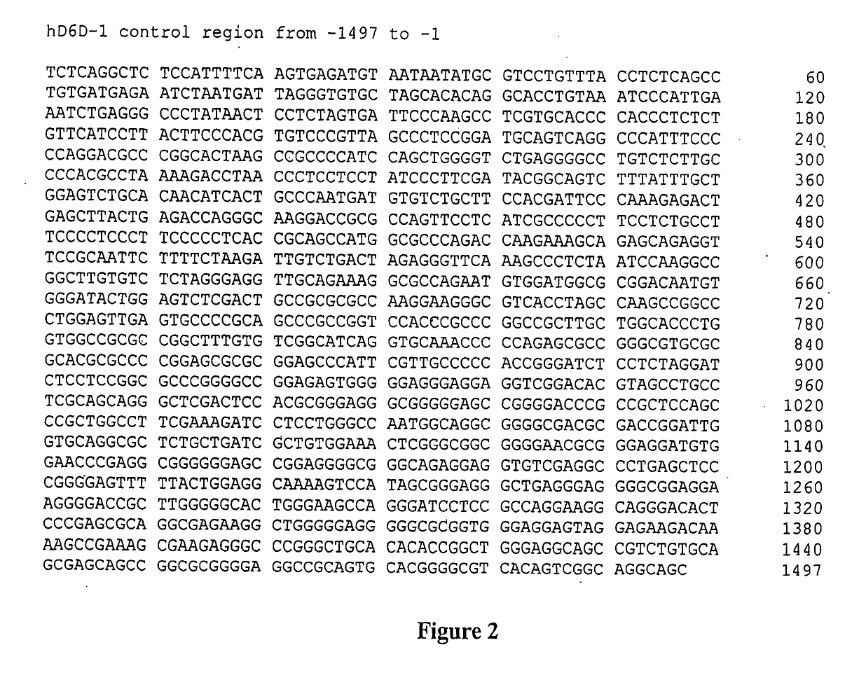

In another embodiment, the invention encompasses novel oligonucleotides comprising the control region of rD6D-1 and hD6D-1 which are represented by the nucleic acid sequences of FIGS. 1 and 2, respectively (SEQ ID NOS:1 and 2).

In yet another embodiment of the invention, a nucleic acid sequence encoding a mammalian desaturase is ligated to a heterologous sequence (e.g. tag or tags) to encode a tagged desaturase. A tagged desaturase is easily identified through the use of an antibody, which will specifically recognize and bind to the heterologous portion of the tagged fatty acid desaturase. Accordingly, a tagged desaturase is beneficial in determining whether the mammalian desaturase has been appropriately expressed in a host system. The carboxyl terminal end of the mammalian desaturase polypeptide is ligated to a stretch of amino acid residues containing tags and in the present invention, is preferably the V5 and the 6×His epitope tags which have the amino acid sequences represented as GKPIPNPLLGLDST and HHHHHH-COOH, respectively. The single-letter code for amino acids used is as follows: A, Ala; C, Cys; D, Asp; E, Glu; F, Phe; G, Gly; H, His; I, Ile; K, Lys; L, Leu; M, Met; N, Asn; P, Pro; Q, Gln; R, Arg; S. Ser; T, Thr, V, Val; W, Trp; Y, Tyr.

Furthermore, a tagged fatty acid desaturase may be engineered to contain a cleavage site located between the desaturase amino acid sequence and the heterologous sequence (e.g. the tag), so that the desaturase may be cleaved away from the heterologous moiety after purification. For example, a system described by Janknecht et al allows for the ready purification of non-denatured tagged proteins expressed in human cell lines (Janknecht et al., 1991, Proc. Natl. Acad Sci, USA, 88: 8972-8976). In this system, the gene of interest is subcloned into a vaccinia recombination plasmid such that the gene's open reading frame is translationally fused to an amino-terminal tag consisting of six histidine residues (6×His). Extracts from cells infected with recombinant vaccinia virus are loaded onto Ni²⁺ nitrilotriacetic acid-agarose columns and histidine-tagged proteins are selectively eluted with imidazole-containing buffers.

Cloning of Mammalian Desaturase Genes and Control Regions

Techniques for cloning, sequencing, expressing and purifying polypeptides are well known to the skilled person. Various techniques are disclosed in standard textbooks, such as Sambrook et al., 1989, Molecular Cloning, A Laboratory Manual, 2^{nd} edition, Cold Spring Harbour Press, Cold Spring Harbour, N.Y.; Old R. W. and Primrose S. B., 1994, Principles of Gene Manipulation, 5th Edition, Blackwell Scientific Publications, U.S.A.; and Ausubel et al., 1994-, Current Protocols in Molecular Biology, John Wiley & Sons, New York, N.Y.

Primers may be designed using the Primer Premier software (Premier Biosoft International, Palo Alto, Calif.), Vector NTI (Informax, Inc., North Bethesda, Md.), OLIGO 4.06 Primer Analysis software (National Biosciences Inc., Plymouth, Minn.), or another appropriate program. Alternatively, the selected primer can be chosen based on cloning strategy without the aid of any software.

Methods for DNA sequencing are well known in the art and employ such enzymes or commercially available kits as SEQUENASE (US Biochemical Corp, Cleveland Ohio)), Taq polymerase (Perkin Elmer, Norwalk Conn.), thermostable T7 polymerase (Amersham, Chicago Ill.), or combinations of recombinant polymerases and proofreading exonucleases such as the ELONGASE amplification system marketed by Gibco BRL (Gaithersburg Md.). Preferably, the process is automated with instruments such as the LiCor DNA Sequencer Long Readir 4200, the Hamilton MICROLAB 2200 (Hamilton, Reno Nev.), Peltier thermal cycler (PTC200; MJ Research, Watertown Me.) or the ABI 377 DNA sequencers (Perkin Elmer).

Mammalian Desaturase Genes

According to the present invention, cDNAs were prepared from mRNA using RT-PCR (reverse transcriptase-polymerase chain reaction) (PCR Protocols: A Guide to Methods and Applications, Innis, M., et al., Academic Press (1990), San Diego, Calif.) employing oligonucleotide forward and reverse primers. Initially, cDNA was generated through reverse transcription of total RNA that was extracted from tissue specific for expressing mammalian fatty acid desaturases using a set of random primers (Perkin-Elmer). Subsequent amplification of desaturase cDNA was achieved by PCR using forward and reverse primers specifically designed to correspond to the coding sequences for the rD6D-1 and hD6D-1 genes, i.e. a forward primer which will hybridize or bind to the 5′-translated region of the antisense strand of the rD6D-1 or hD6D-1 encoding cDNA and a reverse primer which will hybridize or bind to the 3′-translated region of the sense strand of the same desaturase cDNA molecule.

The oligonucleotide primers designed for amplification of mammalian desaturase cDNA may advantageously comprise one or more endonuclease recognition sites to facilitate cloning into an expression vector following amplification by PCP In the present invention, the forward and reverse primers used for cloning the mammalian desaturase genes contain a HindIII and a XbaI restriction site, respectively.

Optionally, an oligonucleotide primer may lack a translation initiation or termination codon so long as such codons are provided in the cloning vector, which need be operatively associated with the cDNA sequence encoding the mammalian desaturase (i.e. positioned upstream at the 5′-end or downstream at the 3′-end of the desaturase encoding sequence, respectively). In a preferred embodiment of the present invention, the translation initiation and termination codons are provided within the forward and reverse primer sequences, respectively, the exception being that the primers used to create the tagged constructs lacked termination codons.

Examples of forward and reverse primers that are useful in cloning rD6D-1 and hD6D-1 cDNAs for insertion into expression vectors are listed below in Table 1. The endonuclease recognition sites are underlined and the translation initiation and termination codons are indicated in boldface type.

TABLE 1

| | |
|---|---|
| Forward - | 5′-CACGCGAAGCTTATGGGGAAGGGAGGTAACCAG-3′ |
| rD6D-1 | |
| Reverse - | 5′-CACGCGTCTAGATCATTTGTGGAGGTAGGCATC-3′ |
| rD6D-1 | |
| Reverse - | 5′-CACGCGTCTAGATTTGTGGAGGTAGGCATCCAG-3′ |
| rD6D-1 | |
| Forward - | 5′-CACGCGAAGCTTATGGGGAAGGGAGGGAAC-3′ |
| hD6D-1 | |
| Reverse - | 5′-CACGACTCTAGAGGGGCTGTGGCTTCATTTGT-3′ |
| hD6D-1 | |
| Reverse - | 5′-CACGCGTCTAGATTTGTGAAGGTAGGCGTCCAG-3′ |
| hD6D-1 | |

In a preferred embodiment of the invention, an rD6D-1 cDNA fragment (1.3 kb) spanning nucleotides +1 to +1335 was cloned by reverse transcription and PCR-amplification of total RNA extracted from rat liver tissue. To this end, the nucleotide sequence that encodes a functionally active rD6D-1 is depicted in FIG. 3. The encoded rD6D-1 is represented by the amino acid sequence depicted in FIG. 5.

In another preferred embodiment of the invention, an hD6D-1 cDNA fragment (1.3 kb) spanning nucleotides +1 to +1335 was cloned from the human cell line Chang (ATCC No.CCl-13) by reverse transcription and PCR-amplification of total RNA. To this end, the nucleotide sequence that encodes a functionally active hD6D-1 is depicted in FIG. 4. The encoded hD6D-1 is represented by the amino acid sequence depicted in FIG. 6.

Mammalian Desaturase Vector Constructs

Methods which are well known to those skilled in the art may be used to construct expression vectors containing nucleic acid sequences encoding mammalian desaturases and appropriate transcriptional and translational control elements. These methods include in vitro recombinant DNA techniques, synthetic techniques, and in vivo genetic recombination. Such techniques are described in Sambrook et al., 1989, Molecular Cloning, A Laboratory Manual, 2^{nd} edition, Cold Spring Harbour Press, Cold Spring Harbour, N.Y. and Ausubel et al., 1994-, Current Protocols in Molecular Biology, John Wiley & Sons, New York, N.Y

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector and/or an expression control sequence, a variety of factors will normally be considered. These include, for example, the relative strength of the expression, its controllability, and its compatibility with the particular nucleic acid sequence or gene to be expressed. Furthermore, in selecting a vector, the host must also be considered because the vector must be maintained and be functional within it. The vector's copy number, the ability to control that copy number, and the expression of any other proteins encoded by the vector, such as antibiotic markers, will also be considered.

The expression control sequence is the non-translated region of the vector (eg. enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the T7 promoter of pET9 (Promega), temperature sensitive promoters, or an osmotically sensitive promoter of pOSEX (Herbst et al., 1994, Gene, 151: 137-142) and the like may be used. The baculovirus polyhedrin promoter and the like may be used in insect cells. Promoters or enhancers derived from the genomes of plant cells (e.g. heat shock, RUBISCO, and storage protein genes) or from plant viruses (e.g., viral promoters or leader sequences) may be cloned into the vector. In mammalian cell systems, promoters from mammalian genes or mammalian viruses are preferable.

Suitable hosts will be selected by consideration of their compatibility with the chosen vector, their secretion characteristics, their ability to fold proteins correctly, the toxicity to the host of the product encoded by the DNA sequences to be expressed, and the ease of purification of the expression products.

Considering these and other factors a person skilled in the art will be able to construct a variety of vector/expression control sequence/host combinations that may be utilized to contain and express nucleic acid sequences encoding mammalian desaturases. Examples of hosts include, but are not limited to, micro-organisms such as bacteria or yeast, insect cell systems; plant cell systems or animal cell systems tansformed/transfected with appropriate expression vectors. A person skilled in the art will be able to introduce the constructs into the appropriate host and propagate the host.

Expression Vectors for Rat and Human Desaturase Genes

In order to express a functionally active mammalian desaturase, the nucleic acid sequence encoding the desaturase is inserted into an appropriate expression vector, i.e. a vector which contains the necessary elements for the transcription and translation of the inserted coding sequence. Depending on the vector system and host utilized, any number of suitable transcription and translation elements may be used.

A range of host systems may be utilized to harbour and express nucleic acid sequences encoding mammalian desaturases. Examples of hosts may include well known prokaryotic hosts, such as strains of E. coli, Pseudomonas putida and Bacillus subtilis; fungi such as yeasts (Saccharomyces cerevisiae, and methylotrophic yeast such as Pichia pastoris, Hansenula polymorpha, Kluyveromyces lactis and Schizosaccharomyces pombe); mammalian cells, such as CHO, African Green Monkey kidney cells (e.g., COS 1, BSC1, BSC40, and BMT10); insect cells (e.g., Sf9); and human cells in tissue culture.

In a preferred embodiment, E. coli is the specific prokaryotic host for cloning and replicating the DNA sequence of the present invention. On the other hand, yeast, in particular Saccharomyces cerevisiae, is the preferred host used for expression of mammalian desaturase coding sequences.

Accordingly, a vector construct of the present invention includes essential elements for its proliferation and selection in both eukaryotic and prokaryotic cells. Preferred expression vectors of the invention are pYES2 and pYES2/CT (Invitrogen) which essentially comprise an origin of replication, an inducible promoter and two selectable marker genes. In particular, the pYES2/CT vector also contains a short DNA sequence that encodes for tags (e.g. V5/6×HIS epitopes) which allow the translated product, a tagged desaturase protein, to be easily identified and/or purified using commercially available antibodies and/or affinity chromatography columns. The pYES2 and pYES2/CT vectors, confer uracil prototrophy for selection in yeast, and a GAL1 galactose-inducible promoter for expression which is activated in the presence of galactose and situated upstream of the cloning site. Galactose-inducible promoters (GAL1, GAL7, and GAL10) have been extensively utilized for high level and regulated expression of proteins in yeast (Lue et al., 1987, Mol. Cell. Biol., 7: 3446-3451 and Johnston M., 1987, Microbiol Rev., 51: 458-476). Transcription from the GAL promoters is activated by the GAL4 protein, which binds to the promoter region and activates transcription when galactose is present. In the absence of galactose, the antagonist GAL80 binds to GAL4 and prevents GAL4 from activating transcription. Addition of galactose prevents GAL80 from inhibiting activation by GAL4.

While it is not essential, optionally an expression vector may comprise a translation initiation or termination (e.g. stop) sequence oriented and operatively associated with the cDNA sequence encoding the mammalian desaturase (i.e. positioned upstream at the 5′-end or downstream at the 3′-end of the desaturase coding sequence, respectively). In a preferred embodiment, the translation initiation and termination codons are already provided within the forward and reverse primer sequences, respectively, which are used to facilitate cloning of the mammalian desaturase genes into the pYES2 vector (see Table 8). Forward and reverse primers for cloning into pYES2/CT are designed to express a desaturase-V5/6×His tagged protein (see Table 8).

The transformed/transfected host cell can be identified by selection for a marker gene contained on the introduced vector construct. The introduced marker gene, therefore, may confer antibiotic resistance, or encode an essential growth factor or enzyme, and permit growth on selective media when expressed in the transformed/transfected host. Typically, transformed/transfected hosts are selected due to their ability to grow on selective media. Selective media may contain an antibiotic or lack an essential growth nutrient necessary for the growth of the untransformed/untransfected host According to the invention, transformation of E. coli cells and yeast cells was determined through selection on ampicillin-containing medium and uracil-deficient medium, respectively, based on the selection marker genes (e.g. beta-lactamase and URA3) present in the pYES2 and pYES2/CT vectors.

A microsomal host system may be achieved by transforming/transfecting the host system with the nucleic acid construct containing the coding sequence for a functional mammalian desaturase described above, and isolating microsomes. Microsomal systems have been used successfully for testing enzyme activity from a number of different sources such as animal organs including liver, brain, heart, etc. and micro-organisms including yeast (de Antueno et al., 1994, Lipids, 29: 327-331, Todd et al., 1999, Plant J., 17: 119-130, and Nishi et al., 2000, Biochim. Biophys. Acta., 1490: 106-108).

Alternatively, an in vitro expression system can be accomplished, for example, by placing the nucleic acid sequence of the coding region for a functional mammalian desaturase polypeptide, described above, in an appropriate expression vector designed for in vitro use. In vitro transcription/translation can be carried out by adding rabbit reticulocyte lysate and essential cofactors; labelled amino acids can be incorporated if desired (Promega Corp., WI). Such in vitro expression vectors may provide some or all of the expression signals necessary in the system used. These methods are well known in the art and the components of the system are commercially available. The reaction mixture can then be assayed directly for the polypeptide, for example by determining its specific enzymatic activity, or the synthesized polypeptide can be purified and then assayed for its specific enzymatic activity.

Reporter Vectors for Rat and Hunan Control Regions

In order to identify the discrete control elements involved in the regulation of mammalian desaturase gene expression, a vector construct comprising a heterologous nucleic acid sequence encoding a reporter gene operably linked to a desaturase control region is used which is compatible to and sufficient for use in a host system.

A range of eukaryotic host systems may be utilized to investigate the activity of the mammalian desaturase control regions. Examples of hosts include, but are not limited to, fungi such as yeasts (Saccharomyces cerevisiae, and methylotrophic yeast such as Pichia pastoris, Hansenula polymorpha, Kluyveromyces lactis and Schizosaccharomyces pombe); mammalian cells, such as HepG2, HeLa, BHK, HEK-293, CHO, African Green Monkey kidney cells (e.g., COS 1, BSC1, BSC40, and BMT10), insect cells (e.g., Sf9), and human cells in tissue culture.

According to the present invention, the preferred cell system used in analysing control regions which are involved in the regulation of the level of mammalian desaturase gene expression is the mammalian cell line ZR-75-1 (ATCC # CRL 1500) or HepG2 (ATCC # HB-8065).

The control region-reporter vector, according to the present invention, can be constructed using conventional molecular biology, microbiology, and recombinant DNA techniques well known to those of skill in the art. Such techniques are explained fully in the literature, including Sambrook et al., 1989, Molecular Cloning, A Laboratory Manual, 2^{nd} edition, Cold Spring Harbour Press, Cold Spring Harbour, N.Y. and Ausubel et al., 1994-, Current Protocols in Molecular Biology, John Wiley & Sons, New York, N.Y.

The practice of using a reporter gene to analyse nucleotide sequences which regulate transcription of genes involved in PUFA metabolism is well documented (Water K. M., 1997, Biochim. Biophys. Acta., 1349: 33-42). Generally, a reporter gene encodes a polypeptide not otherwise produced by the host cell and which is detectable by analysis of the host cell. The product of a reporter gene is used to assess regulation of transcription via a control region/oligonucleotide sequence of the present invention. The expression of the reporter gene results in the formation of a reporter product (e.g. protein) which is readily detectable and hence, has a utility in its quantitative and/or qualitative capability to demonstrate that transcriptional activation has occurred. The reporter gene will be selected such that the reporter product will have physical and chemical characteristics, which facilitate its identification or detection, by means well known in the art. Reporter genes which are widely utilized in such studies include, but are not limited to, enzymes such as luciferase, chloramphenicol acetyl transferase (CAT), beta-galactosidase, esterases, phosphatases, proteases and other proteins such as green fluorescence protein (GFP) and human growth hormone. In a preferred embodiment, the reporter gene is CAT which will be detected through the level of specific enzymatic activity, which in turn correlates to the amount of enzyme that was made and hence, the level of expression of the reporter gene.

A reporter vector construct of the present invention includes essential elements for its propagation, selection and expression in either prokaryotic or eukaryotic cells.

The reporter vector of the present invention, which includes essential elements for its operability in prokaryotic or eukaryotic cells, is, preferably, pCAT-3-Enhancer or pGL3-Basic (Promega Corp., WI). The mammalian desaturase control region, derived from genomic DNA, is ligated by conventional methods in proper orientation (5′ to 3′) adjacent (5′) to the start codon of the reporter gene with or without additional control elements. The region 3′ to the coding sequence for the reporter gene will contain a transcription termination and polyadenylation site, for example, the SV40 polyA site. The desaturase control region and reporter gene, which are operably linked in the reporter vector, are transformed into a cloning host, preferably E. coli. The host is cultured and the replicated vector recovered in order to prepare sufficient quantities of the recombinant construction for subsequent transfection into a second host, preferably the mammalian cell line ZR-75-1 or HepG2.

Alternatively, an in vitro expression system can be accomplished, for example, by placing the nucleic acid sequence for a mammalian control region, described above, in an appropriate reporter vector designed for in vitro use. In vitro transcription can be carried out by adding nuclear extract from mammalian cells and other necessary reagents. Such in vitro reporter vectors may provide some or all of the expression signals necessary in the system used. These methods are well known in the art.

Accordingly, a vector construct of the present invention includes essential elements for its proliferation and selection in both eukaryotic and prokaryotic cells. Expression vectors of the invention include pYES2 and pYES2/CT (Invitrogen) which essentially comprise an origin of replication, an inducible promoter and two selectable marker genes. In particular, the pYES2/CT vector also contains a short DNA sequence that encodes for tags (e.g. V5/6×His epitopes) which allow the translated product, a tagged desaturase protein, to be easily identified and/or purified using commercially available antibodies and/or affinity chromatography columns. The pYES2 and pYES2/CT vectors, confer uracil prototrophy for selection in yeast, and a GAL1 galactose-inducible promoter for expression which is activated in the presence of galactose and situated upstream of the cloning site. Galactose-inducible promoters (GAL1, GAL7, and GAL 10) have been extensively utilized for high level and regulated expression of proteins in yeast (Lue et al., 1987, Mol. Cell. Biol, 7: 3446-3451 and Johnston M., 1987, Microbiol Rev., 51: 458-476). Transcription from the GAL promoters is activated by the GAL4 protein, which binds to the promoter region and activates transcription when galactose is present In the absence of galactose, the antagonist GAL80 binds to GAL4 and prevents GAL4 from activating transcription. Addition of galactose prevents GAL80 from inhibiting activation by GAL4.

An expression vector may comprise a translation initiation or termination (e.g. stop) sequence oriented and operatively associated with the cDNA sequence encoding the mammalian desaturase (i.e. positioned upstream at the 5′-end or downstream at the 3′-end of the desaturase coding sequence, respectively). However, the translation initiation and termination codons may be already provided within the forward and reverse primer sequences, respectively, which are used to facilitate cloning of the mammalian desaturase genes into the pYES2 vector (see Example 5). Forward and reverse primers for cloning into pYES2/CT are designed to express a desaturase-V5/6×His tagged protein (see Example 5).

Thus, according to one aspect of the invention, there is provided a recombinant nucleic acid construct which contains a portion of a mammalian desaturase gene comprising the amino acid coding region and which has a heterologous promoter capable of initiating transcription of a fatty acid desaturase gene. In preferred embodiments of the invention, the amino acid coding region is derived from a human or a rat desaturase gene. In particular, the invention provides a nucleic acid construct having a promoter region which is preferably induced, a nucleic acid sequence encoding a functional mammalian (e.g. human or rat) fatty acid desaturase and a termination region, whereby the promoter region is operably associated with the nucleic acid sequence so as to effectively control expression of the nucleic acid sequence. Alternatively, the recombinant nucleic acid construct may comprise a heterologous transcriptional termination region functional in a host system. The recombinant nucleic acid construct is cloned as part of an expression vector, which can then be inserted into a host system.

In another embodiment of the invention, a polynucleotide encoding a mammalian (e.g. human or rat) fatty acid desaturase may be ligated to a heterologous sequence to encode a tagged protein. For example, for screening of host systems for proteins exhibiting fatty acid desaturase activity, it may be useful to encode a tagged desaturase protein that is recognized by a commercially available antibody. A tagged protein may also be engineered to contain a cleavage site located between a desaturase coding sequence and the heterologous protein sequence, so that the fatty acid desaturase may be cleaved and purified away from the heterologous moiety.

Another aspect of the present invention is directed to a recombinant nucleic acid construct containing a control region of a mammalian fatty acid desaturase gene and a reporter gene. In preferred embodiments of the invention, the control region is derived from a human or a rat desaturase gene. The control region and the reporter sequence are operably linked so that the control region can effectively initiate, terminate or regulate the transcription or translation of the reporter sequence. The recombinant nucleic acid construct is cloned as part of an expression vector, which can then be inserted into a host system.

Host Systems

The invention provides a recombinant nucleic acid construct which contains a portion of a mammalian D6D gene including the amino acid coding region and which has a heterologous promoter capable of initiating transcription of a fatty acid desaturase gene. The amino acid coding region is derived from a human D6D gene. In particular, the invention provides a nucleic acid construct having a heterologous promoter region which is preferably induced, a nucleic acid sequence encoding a functional mammalian (e.g. human or rat) D6D and a termination region, whereby the promoter region is operably associated with the nucleic acid sequence so as to effectively control expression of the nucleic acid sequence. Alternatively, the recombinant nucleic acid construct may comprise a heterologous transcriptional termination region functional in a host system. The recombinant nucleic acid construct is cloned as part of an expression vector, which can then be inserted into a host system.

A polynucleotide encoding a mammalian (e.g. human or rat) D6D gene may be ligated to a heterologous sequence to encode a tagged protein. For screening of host systems that express D6D, it may be useful to encode a tagged desaturase protein that is recognized by a commercially available antibody. A tagged protein may also be engineered to contain a cleavage site located between a D6D coding sequence and the heterologous protein sequence, so that the fatty acid desaturase is cleaved and purified from the heterologous moiety.

Another aspect of the present invention is directed to a recombinant nucleic acid construct containing a control region of a mammalian D6D gene and a reporter gene. The control region is derived from a human D6D gene. The control region and the reporter sequence are operably linked so that the control region effectively initiates, terminates or regulates the transcription or translation of the reporter sequence. The recombinant nucleic acid construct is cloned as part of an expression vector, which is then inserted into a host system.

Accordingly, the host system is transformed/transfected by the nucleic acid construct containing the nucleic acid sequence of the D6D gene such that the promoter region and the termination region are operable and can, therefore, be used to achieve high level expression of a functionally active desaturase enzyme. A test component, which increases or decreases desaturase enzyme activity, is an enhancer or inhibitor, respectively. Consequently, defined test components can be used as a basis for the formulation or innovation of therapeutic agents to treat disease related to the level of active and regulated D6D enzyme in tissue.

The tansformed/transfected host cell is identified by selection for a marker gene contained on the introduced vector construct. The introduced marker gene, therefore, may confer antibiotic resistance, or encode an essential growth factor or enzyme, and permit growth on selective media when expressed in the transformed/transfected host. Typically, transformed/transfected hosts are selected due to their ability to grow on selective media. Selective media may contain an antibiotic or lack an essential growth nutrient necessary for the growth of the untransformed/untransfected host. Transformation of Escherichia coli cells and yeast cells was determined through selection on ampicillin-containing medium and uracil-deficient medium, respectively, based on the selection marker genes (e.g. beta-lactamase and URA3) present in the pYES2 and pYES2/CT vectors.

A cell-free expression system is achieved by placing the nucleic acid construct, comprising the coding sequence for a functional mammalian desaturase described above, into an appropriate expression vector designed for in vitro use and carrying out in vitro transcription/translation in a cell lysate, such as mRNA-dependent rabbit reticulocyte lysate. If required, additional components may be incorporated into the system such as essential co-factors and amino acids. Microsomal systems have been used successfully for testing enzyme activity from a number of different sources such as animal organs including liver, brain, heart, etc. and micro-organisms including yeast (de Antueno et al., 1994, Lipids, 29: 327-331; Todd et al., 1999, Plant J., 17: 119-130; and Nishi et al., 2000, Biochim. Biophys. Acta, 1490: 106-108).

A microsomal host system is achieved by transforming/transfecting the host system with the nucleic acid construct containing the coding sequence for a functional mammalian desaturase described above, and isolating microsomes (Ausubel et al., 1994-, Current Protocols in Molecular Biology, John Wiley & Sons, New York, N.Y.). In vitro transcription/translation is carried out by adding rabbit reticulocyte lysate and essential cofactors; labelled amino acids can be incorporated if desired. Such in vitro expression vectors may provide some or all of the expression signals necessary in the system used. These methods are well known in the art and the components of the system are commercially available. The reaction mix is assayed directly for the polypeptide, for example by determining its specific enzymatic activity, or the synthesized polypeptide purified and then assayed for its specific enzymatic activity.

A cell system used in analyzing control regions which are involved in the regulation of the level of mammalian D6D gene expression is the mammalian cell lines ZR-75-1 (ATCC No. CRL-1500) or HepG2 (ATCC No. HB-8065).

Reporter genes which are widely utilized in such studies include, but are not limited to, enzymes such as luciferase, chloramphenicol acetyl transferase (CAT), beta-galactosidase, esterases, phosphatases, proteases and other proteins such as green fluorescence protein (GFP) and human growth hormone. In preferred embodiments, the reporter gene is either CAT or luciferase which will be detected through the level of specific enzymatic activity, which in turn correlates to the amount of enzyme that was made and hence, the level of expression of the reporter gene.

A reporter vector of the present invention, which includes essential elements for its operability in prokaryotic or eukaryotic cells, is pCAT-3-Basic (Promega Corp., WI). The mammalian desaturase control region, derived from genomic DNA, is ligated by conventional methods in proper orientation (5′ to 3′) adjacent (5′) to the start codon of the reporter gene with or without additional control elements. The region 3′ to the coding sequence for the reporter gene contains a transcription termination and polyadenylation site, for example, the SV40 polyA site. The desaturase control region and reporter gene, which are operably linked in the reporter vector, are transformed into a cloning host, preferably E. coli. The host is cultured and the replicated vector recovered in order to prepare sufficient quantities of the recombinant construction for subsequent transfection into a second host, preferably the mammalian cell lines ZR-75-1 or HepG2.

Drug Screening Assays

When a preferred host cell is transfected or transformed with a DNA construct according to the present invention, it can be utilized in assays to identify potential test components that can modulate desaturase enzyme activity or alter the level of desaturase gene transcription via regulatory elements/oligonucleotide sequences. The screening assay typically is conducted by (1) growing the host cells transformed or transfected with desaturase genes or control regions to a suitable state of confluency in appropriate plates or flasks (e.g., microtiter wells, Erlenmeyers, etc.), (2) adding the test components to a series of wells or flasks, and (3) determining the signal level (e.g. desaturase activity or level of gene expression) after an incubation period that is suitable to demonstrate a measurable signal in the assay system chosen. The wells or flasks, containing varying proportions and/or classes of test components can be evaluated by signal activation within the treated cells. Candidates that demonstrate modulation of desaturase enzyme activity or reporter gene expression are then selected for further evaluation as clinical therapeutic agents.

A host cell strain may be chosen for its ability to modulate the expression of the inserted sequences, or to process the gene product in the desired fashion. Such modifications (e.g. glycosylation) and processing (e.g. cleavage) of protein products may be important for the function of the protein. Different host cells have characteristic and specific mechanisms for the post-translational processing and modification of proteins and gene products which may also be important to ensure correct processing and functioning of the expressed foreign protein. Appropriate cell lines or host systems can be chosen to ensure the correct modification and processing of the foreign protein expressed. To this end, prokaryotic or eukaryotic host cells, which possess the cellular machinery for proper processing of the primary transcript, and for proper glycosylation, phosphorylation and folding of the gene product may be used. Such prokaryotic, or eukaryotic host cells include but are not limited to E. coli, Bacillus subtilis, Pseudomonas putida, Saccharomyces cerevisiae, Schizosaccharomyces pombe, Pichia pastoris, Hansenula polymorpha, Kluyveromyces lactis, ZR-75-1, Chang, CHO, VERO, BHK, HeLa, COS, MDCK, 293, 3T3, WI38, and U937 cells.

In a preferred embodiment of the present invention, the medium for conducting the drug screening method is an eukaryotic cell, including fungal and mammalian cells.

Modulation of Mammalian Desaturase Activity

More specifically, an embodiment of the present invention relates to a drug screening assay using transformed yeast as whole cells, spheroplasts, cell homogenates or organelles (e.g. microsomes, etc.) to identify candidate agents that modulate the enzymatic activity of a mammalian desaturase. In a preferred embodiment of the present invention the host yeast Saccharomyces cerevisiae, strain INVSc1, Invitrogen San Diego, Calif.) is transformed with the yeast expression vector, pYES2 (Invitrogen), containing the mammalian desaturase coding sequence. Yeast cells are selected for use in the present method because (1) they have not shown fatty acid delta-6-desaturase activity, (Aki et al, 1999), (2) the transcription and translation processes are similar, if not identical, to processes that occur in mammalian cells, and (3) yeast cells are often more amenable to genetic manipulation than mammalian cells, and they grow much more rapidly (Guthrie C. and Fink G., 1991, Methods in Enzymology, 194). Thus, yeast cells provide an excellent model for eukaryotic gene expression and for studying the modulation of mammalian desaturase activity.

When a preferred host cell, such as a yeast cell, is transformed with a DNA construct according to the present invention, it can be utilized in assays to identify potential test components that can modulate desaturase activity. Test components having the potential to modulate desaturase activity can be identified by (1) contacting the transformed host cell with the test component for a fixed period of time, and (2) determining the level of lipid metabolite (e.g. the level of product produced from substrate) within the treated cells. This level of metabolite in one cell can then be compared to the level of metabolite in the absence of the test component. The difference between the levels of metabolite, if any, indicates whether the test component of interest modulates desaturase activity. Furthermore, the magnitude of the level of lipid metabolite generated between the treated and untreated cells provides a relative indication of the strength of that compound(s) as a modulator of desaturase activity. Rat liver microsomes (obtained as described in other Examples) may be used in conjunction with the preferred host system to corroborate the strength of that compound(s) as a modulator of desaturase activity.

Modulation of Mammalian Desaturase Gene Expression

The present invention also relates to a drug screening assay using mammalian cells as host systems to observe the regulation of desaturase gene expression and identify test components that modulate the expression of a reporter gene driven by desaturase gene control regions or regulatory elements. In a preferred embodiment of the present invention, the ZR-75-1 (human mammary carcinoma) cell line is used as the host system which is transfected with the reporter vector, pCAT-3-Enhancer (chloramphenicol acetyl transferase; Promega Corp., WI) containing the mammalian desaturase control sequence. ZR-75-1 cells were selected for use in the present method because (1) this cell line shows high level of delta-6-desaturase mRNA expression (as shown by Northern blot), and (2) it is amenable for transfection. Alternatively, the HepG2 cell line was used as the host system for stable transfection of the hD6D control region inserted in the pGL3-Basic reporter vector (luciferase; Promega Corp., WI). This human hepatoma cell line was chosen to study regulation of the D6D promoter activity because of previous gene expression results (Northern blots) which indicate regulation of the D6D gene in this cell line that is similar to what was seen in rat liver.

When a preferred host cell line, such as ZR-75-1 or HepG2, is transfected with a reporter DNA construct according to the present invention, it can be utilized in assays to identify potential test components that can modulate the level of gene transcription via functionally active regulatory elements/oligonucleotide sequences. Test components having the potential to alter the level of gene transcription can be identified by (1) contacting the transfected host cell with the test component for a fixed period of time, and (2) determining the level of gene expression (e.g. the level of CAT produced) within the treated cells. This expression level can then be compared to the expression level of the reporter gene in the absence of the compound(s). The difference between the levels of gene expression, if any, indicates whether the compound(s) of interest modifies the functionality of the DNA regulatory elements. Furthermore, the magnitude of the level of reporter product expressed between the treated and untreated cells provides a relative indication of the strength of that compound(s) as a modulator of the desaturase gene transcription via transcriptional DNA regulatory elements.

Host Systems and Drug Screening

The invention includes methods for screening nucleotides, proteins, compounds or pharmacological agents, which enhance or inhabit D6D gene expression at the transcriptional level or modulate the D6D activity. To this end, cell-based, cell lysate and/or purified enzyme assays are used to detect these enhancing or inhibiting components.

D6D gene expression has been associated with diabetes and related disorders, arterial hypertension; hypercholesterolemia; atherosclerotic heart disease; chronic inflammatory disorders; autoimmune disorders; allergic eczema and other atopic disorders; inflammatory processes such as rheumatoid arthritis; diminished lymphocyte proliferation, T-cell-mediated cytotoxicity, natural killer cell activity, macrophage-mediated cytotoxicity, monocyte and neutrophil chemotaxis, major histocompatibility class II expression and antigen presentation, production of pro-inflammatory cytokines (interleukins 1 and 6, tumour necrosis factor) and adhesion molecule expression; eczema; psoriasis; acute respiratory distress syndrome (ARDS); articular cartilage degradation (ACD); and cancer.

A present inventors' human diabetic clinical trial has provided data indicating that AA and EPA were reduced in the plasma and red cell phospholipids of Type 1 diabetics. This study supports and expands a multi-center clinical trial sponsored by Scotia Pharmaceuticals in which enteral administration of n-6 PUFAs ameliorates neurophysiological parameters of mild diabetic neuropathy (Keen et al., 1993, Diabetes Care, 16: 8-15). Reduced levels of long chain n-6 fatty acids have been reported (Arisaka et al., 1986, J. Paediatr. Gastroenterol. Nutr., 5: 878-882; Tilvis R. S. and Miettinen T. A., 1985, J. Clin. Endocrinol. Metab., 61: 741-745; and van Doormaal et al., 1988, Diabetologia, 31: 576-584). The level of DGLA was not reduced in the Type 1 diabetic group, indicating that the reduction of AA may be due to reduced delta-6-desaturase activity.

In a present inventors' diabetic rat study, the plasma phospholipid AA content was reduced 31% and 27% in the 2 week and 7 week streptozotocin-induced diabetic rats, respectively. As in the human diabetic study, the DGLA levels remained unchanged compared to controls, so the reduced levels of AA and EPA were consistent with a detected reduction in delta-6-desaturase activity. Reduced activity of the desaturase system in diabetes was first reported by Brenner et al., 1968, Am. J. Physiol., 215: 63-70. Subsequently, this finding has been verified (Mimouni V. and Poisson J. P., 1992, Biochim. Biophys. Acta, 1123: 296-302; Dang et al., 1989, Lipids, 24: 882-889; and Faas F. H. and Carter W. J., 1980, Lipids, 15: 953-961) and is considered to be a key factor in the development of secondary complications of diabetes. In the streptozotocin diabetic rat study, it was determined that the delta-6-desaturase activity in hepatic microsomes from diabetic rats was reduced by 37% compared to the control rats. These findings support the hypothesis that delta-6-desaturase is a potential drug target in diabetes and also a useful lipid metabolic compound for drug screening assays.

The present invention features a drug screening method for identifying nucleotides, proteins, compounds, and/or pharmacological agents which modulate or regulate the transcription of a mammalian D6D gene. This method includes (1) providing a novel nucleic acid construct having a control region of a mammalian desaturase gene and a heterologous nucleic acid sequence (e.g. a reporter gene), wherein the control region is operably associated with the nucleic acid sequence so that it effectively initiates, terminates or regulates the transcription of the nucleic acid sequence, all of which are introduced into a cell or cell lysate using an expression vector containing the novel nucleic acid construct, (2) contacting the cell or cell lysate with a test component, (3) determining whether the test component is capable of altering the level of transcription of the nucleic acid sequence, and (4) selecting those components which exhibit such activity. In this regard, the defined test components can be used as a basis for the formulation or innovation of therapeutic drugs to treat disease related to the level of D6D gene expression. Test components, which increase or decrease the level of transcription of the reporter sequence, are enhancers or inhibitors, respectively.

In particular, the present invention embodies a method for the identification of useful and functional portions of the D6D control region and various functional and regulatory elements within the control region, which are associated with the level of expression of the desaturase gene. Functional portions of the desaturase control region which result in altered levels of gene expression are determined through the manipulation (e.g. deletion, site-directed mutagenesis, etc.) of various segments of the region, as well as through the direct or indirect effect of modulators.

The host system for conducting the drug screening method can be eukaryotic cells, including fungal or mammalian cells. More specifically, an embodiment of the present invention relates to a drug screening assay using transformed yeast as whole cells, spheroplasts, cell homogenates, organelles (e.g. microsomes, etc.) or purified enzyme to identify candidate agents that modulate the enzymatic activity of a mammalian D6D. In an embodiment of the present invention the host yeast Saccharomyces cerevisiae, strain INVSc1 (Invitrogen, CA), is transformed with the yeast expression vectors, pYES2 or pYES2/CT (Invitrogen), containing the mammalian D6D coding sequence. Yeast cells are selected for use in the present method because (1) they have not shown fatty acid delta-6-desaturase activity (Aki et al, 1999, Biochem. Biophys. Res. Commun., 255: 575-579), (2) their transcription and translation processes are similar, if not identical, to processes that occur in mammalian cells, and (3) they are often more amenable to genetic manipulation than mammalian cells, and they grow much more rapidly (Guthrie C. and Fink G., 1991, Meth. Enzymol, 194). Thus, yeast cells provide an excellent model for eukaryotic gene expression and for studying the modulation of mammalian D6D activity.

When a host cell, such as a yeast cell, is transformed with a DNA construct according to the present invention, it is utilized in assays to identify test components that modulate desaturase activity. Test components that modulate D6D activity are identified by (1) contacting the transformed host cell with the test component for a fixed period of time, and (2) determining the level of lipid metabolite (i.e. the level of product produced from substrate) or associated cofactors within the treated cells. This level of metabolite in one cell can then be compared to the level of metabolite in the absence of the test component. The difference between the levels of metabolite, if any, indicates whether the test component of interest modulates D6D activity. Furthermore, the magnitude of the level of lipid metabolite generated between the treated and untreated cells provides a relative indication of the strength of that compound(s) as a modulator of desaturase activity. Rat liver microsomes are used in conjunction with the preferred host system to corroborate the strength of that compound(s) as a modulator of desaturase activity.

A drug screening assay is also carried out using mammalian cells as host systems to observe the regulation of D6D gene expression and identity test components that modulate the expression of a reporter gene driven by D6D gene control regions or regulatory elements. ZR-75-1 or HepG2 cell lines are preferably used as the host systems, which are transfected with the reporter vectors, pCAT-3-Basic (Promega) or pGL3-Basic (Promega) containing the mammalian D6D control sequence.

When a preferred host cell line, such as ZR-75-1, is transfected with a reporter DNA construct according to the present invention, it is utilized in assays to identify test components that modulate the level of gene transcription via functionally active regulatory elements/oligonucleotide sequences.

Test components that alter the level of gene transcription can be identified by (1) contacting the transfected host cell with the test component for a fixed period of time, and (2) determining the level of gene expression (e.g. CAT activity) within the treated cells. This expression level is compared to that of the reporter gene in the absence of the compound(s). The difference between the levels of gene expression, if any, indicates whether the compound(s) of interest modifies the functionality of the DNA regulatory elements. Furthermore, the magnitude of the level of reporter product expressed between the treated and untreated cells provides a relative indication of the strength of that compound(s) as a modulator of the D6D gene transcription via transcriptional DNA regulatory elements.

In an embodiment, a high-throughput screening protocol is used to survey a large number of test compounds for their ability to modulate or regulate the transcription of a mammalian D6D gene through their effect on the desaturase control region. Accordingly, the design of the transcriptional system makes it possible to screen a large selection of components as potential therapeutic agents that alter D6D gene expression thereby increasing or decreasing tissue levels of a functional D6D enzyme, the physiological significance of which includes the normalization of lipid metabolites.

For the drug screening methods described herein, the host system may be a cell, tissue, organ, organism or any part thereof, which provides an environment or conditions that allow for, or enable, transcription and/or transcription followed by subsequent translation to yield a functional protein or polypeptide. Organisms would include animals such as mammals. In an embodiment of the invention, the drug screening methods are conducted in prokaryotic and eukaryotic cells. In embodiments of the invention, the eukaryotic cells include yeast cells and mammalian cells.

Potential antagonists include small organic molecules, peptides, polypeptides and antibodies that bind to a polynucleotide or polypeptide of the invention and thereby inhibit or extinguish its activity. Potential antagonists also may be small organic molecules, a peptide, a polypeptide such as a closely related protein or antibody that bind the same sites on a binding molecule, such as a binding molecule, without inducing delta-6-desaturase-induced activities, thereby preventing the action of delta-6-desaturase by interfering with substrate binding.

Potential antagonists include a small molecule, which bind to and occupy the binding site of the polypeptide thereby preventing binding to cellular binding molecules, such that normal biological activity is prevented. Examples of small molecules include but are not limited to small organic molecules, peptides or peptide-like molecules. Other potential antagonists include antisense molecules (see Okano et al., 1988, EMBO J., 7: 3407-3412 for a description of these molecules).

Selective modulators may include, for example, antibodies and other proteins or peptides which specifically bind to the delta-6-desaturase or delta-6-desaturase nucleic acid, oligonucleotides which specifically bind to delta-6-desaturase (see Patent Cooperation Treaty International Publication No. WO93/05182 published Mar. 18, 1993) which describes methods for selecting oligonucleotides which selectively bind to target biomolecules) or delta-6-desaturase nucleic acid (e.g. antisense oligonucleotides) and other non-peptide natural or synthetic compounds which specifically bind to the delta-6-desaturase or delta-6-desaturase nucleic acid.

Targets for the development of selective modulators include, for example: (1) the regions of the delta-6-desaturase which contact other proteins and/or localize the delta-6-desaturase within a cell and (2) the regions of the delta-6-desaturase which bind substrate.

Thus, according to another aspect of the invention there is provided a drug screening method for identifying nucleotides, proteins, compounds and/or pharmacological agents that effectively modulate the activity of fatty acid desaturase enzymes and hence, fatty acid profiles. The method comprises (1) producing a nucleic acid construct having a promoter region, which is preferably induced, a nucleic acid sequence encoding a functional fatty acid desaturase enzyme, whereby the promoter region is operably associated with the nucleic acid sequence, and a termination sequence, all of which are introduced into a cell or cell lysate using an expression vector containing the nucleic acid construct, (2) contacting the cell or cell lysate with a test component, (3) evaluating the enzymatic activity of a desaturase polypeptide encoded by the nucleic acid sequence by assaying for a measurable difference in the level of lipid metabolite as an indicator of the ability of the test component to modulate fatty acid desaturase enzyme activity, and (4) selecting those components which exhibit such activity. The known substrate for the fatty acid desaturase may optionally be exogenously supplied to the cell or cell lysate.

Accordingly, the host system is transformed/transfected by the nucleic acid construct containing the nucleic acid sequence of the fatty acid desaturase gene such that the promoter region and the termination region are operable and can, therefore, be used to achieve high level expression of a functionally active desaturase enzyme. A test component which increases or decreases desaturase enzyme activity is an enhancer or inhibitor, respectively. Consequently, defined test components can be used as a basis for the formulation or innovation of therapeutic agents to treat disease related to the level of active and regulated fatty acid desaturase enzymes in tissue.

A microsomal host system may be achieved by transforming/transfecting the host system with the nucleic acid construct containing the coding sequence for a functional mammalian desaturase described above, and isolating microsomes (Ausubel et al., 1994-, Current Protocols in Molecular Biology, John Wiley & Sons, New York, N.Y.).

A cell-free expression system may be achieved by placing the nucleic acid construct comprising the coding sequence for a functional mammalian desaturase described above, inserting it into an appropriate expression vector designed for it vitro use and carrying out in vitro transcription/translation in a cell lysate, such as mRNA-dependent rabbit reticulocyte lysate. If required, additional components may be incorporated into the system such as essential co-factors and amino acids.

In a preferred embodiment, a high-throughput screening protocol is used to survey a large number of test compounds for their ability to modulate the enzymatic activity of a mammalian fatty acid desaturase. Accordingly, the design of the drug screening method makes it possible to screen a large selection of components as potential therapeutic agents that alter fatty acid desaturase activity thereby increasing or decreasing levels of specific lipid metabolites, the physiological significance of which includes the normalization of lipid metabolism.

In another aspect, the present invention features a drug screening method for identifying nucleotides, proteins, compounds, and/or pharmacological agents which modulate or regulate the transcription of a mammalian fatty acid desaturase gene. This method includes (a) providing a novel nucleic acid construct having a control region of a mammalian desaturase gene and a heterologous nucleic acid sequence (e.g. a reporter gene), wherein the control region is operably associated with the nucleic acid sequence so that it can effectively initiate, terminate or regulate the transcription of the nucleic acid sequence, all of which are introduced into a cell or cell lysate using an expression vector containing the novel nucleic acid construct, (b) contacting the cell or cell lysate with a test component, (c) determining whether the test component is capable of altering the level of transcription of the nucleic acid sequence, and (d) selecting those components which exhibit such activity. In this regard, the defined test components can be used as a basis for the formulation or innovation of therapeutic drugs to treat disease related to the level of fatty acid desaturase gene expression. Test components, which increase or decrease the level of transcription of the reporter sequence, are enhancers or inhibitors, respectively.

In particular, the present invention embodies a method for the identification of useful and functional portions of the fatty acid desaturase control region and various functional and regulatory elements within the control region which are associated with the level of expression of the desaturase gene. Functional portions of the desaturase control region which result in altered levels of gene expression are determined through the manipulation (e.g. deletion, site-directed mutagenesis, etc.) of various segments of the region, as well as through the direct or indirect effect of modulators.

A cell-free expression system may be achieved by placing the novel nucleic acid construct comprising the control region of a mammalian desaturase gene and a reporter sequence as described above, inserting it into an appropriate expression vector designed for in vitro use and carrying out in vitro expression in a cell lysate. If required, additional components may be incorporated into the system such as essential co-factors and other reagents.

In a preferred embodiment, a high-throughput screening protocol is used to survey a large number of test compounds for their ability to modulate or regulate the transcription of a mammalian fatty acid desaturase gene through their effect on the desaturase control region. Accordingly, the design of the transcriptional system makes it possible to screen a large selection of components as potential therapeutic agents that alter fatty acid desaturase gene expression thereby increasing or decreasing tissue levels of a functional desaturase enzyme, the physiological significance of which includes the normalization of lipid metabolites.

For the drug screening methods described above, the host system may be a cell, tissue, organ, organism or any part thereof, which provides an environment or conditions that allow for, or enable, transcription and/or transcription followed by subsequent translation to yield a functional protein or polypeptide. Organisms would include animals such as mammals. In a preferred embodiment of the invention, the drug screening methods are conducted in prokaryotic and eukaryotic cells. In preferred embodiments of the invention, the eukaryotic cells include yeast cells and mammalian cells.

Drug Design

Modulation of delta-6-desaturase gene function can be accomplished by the use of therapeutic agents or drugs which can be designed to interact with different aspects of delta-6-desaturase control region structure or function. For example, a drug or antibody can bind to a structural fold of the control region to correct a defective structure. Alternatively, a drug might bind to a specific functional residue and increase its affinity for a substrate or cofactor. Efficacy of a drug or agent can be identified by a screening program in which modulation is monitored in vitro in cell systems in which a delta-6-desaturase gene protein is expressed. Alternatively, drugs can be designed to modulate delta-6-desaturase gene activity from knowledge of the structure and function correlations and from knowledge of the specific defect in the various NF1 mutant proteins (see Copsey D. N. and Delnatte S. Y. J., 1988, Genetically Engineered Human Therapeutic Drugs, Stockton Press, New York).

Gene Therapy

A variety of gene therapy approaches may be used in accordance with the invention to modulate expression of delta-6-desaturase in vivo. For example, antisense DNA molecules may be engineered and used to block delta-6-desaturase DNA in vivo. In another alternative, oligonucleotides designed to hybridize to the 5′ region of the delta-6-desaturase control sequence and form triple helix structures may be used to block or reduce transcription of the delta-6-desaturase. In yet another alternative, nucleic acid encoding the full length wild-type delta-6-desaturase control region may be introduced in vivo into cells which otherwise would be unable to produce the wild-type delta-6-desaturase product in sufficient quantities or at all.

For example, in conventional replacement therapy, gene product or its functional equivalent is provided to the patient in therapeutically effective amounts. Delta-6-desaturase protein can be purified using conventional techniques such as those described in Deutcher, M. (editor), 1990, Guide to Protein Purification. Meth.Enzymol.: 182. Sufficient amounts of gene product or protein for treatment can be obtained, for example, through cultured cell systems or synthetic manufacture. Drug therapies which stimulate or replace the gene product can also be employed. Delivery vehicles and schemes can be specifically tailored to the particular protein or drug being administered.

Gene therapy using recombinant technology to deliver the gene into the patient's cells or vectors, which will supply the patient with gene product in vivo, is also contemplated as within the scope of the present invention. Retroviruses have been considered a preferred vector for experiments in somatic gene therapy, with a high efficiency of infection and stable integration and expression (Orkin, et al., 1988, Prog. Med. Genet. 7: 130-142). For example, delta-6-desaturase cDNA can be cloned into a retroviral vector and driven from either its endogenous promoter of from the retroviral LTR (long terminal repeat). Other delivery systems which can be utilized include adeno-associated virus (AAV) (McLaughlin et al., 1988, J. Virol 62: 1963-1973), vaccinia virus (Moss et al., 1987, Annu. Rev. Immunol. 5: 305-324), bovine papilloma virus (Rasmussen, et al.,1987, Meth. Enzymol. 139: 642-654), or member of the herpesvirus group such as Epstein-Barr virus (Margolskee, et al., 1988, Mol Cell. Biol. 8: 2837-2847.

In another embodiment, the antisense, ribozyme and triple helix nucleotides are designed to inhibit the translation or transcription of delta-6-desaturase. To accomplish this, the oligonucleotides used should be designed on the basis of relevant sequences unique to delta-6-desaturase control region.

For example, and not by way of limitation, the oligonucleotides should not fall within those region where the nucleotide sequence of a subject polynucleotide is most homologous to that of other fatty acid enzyme polynucleotides, herein referred to as “unique regions”.

In the case of antisense molecules, it is preferred that the sequence be chosen from the unique regions. It is also preferred that the sequence be at least 18 nucleotides in length in order to achieve sufficiently strong annealing to the target mRNA sequence to prevent translation of the sequence. Izant J. G. and Weintraub H., 1984, Cell, 36: 1007-1015; Rosenberg et al., 1985, Nature, 313: 703-706.

In the case of the “hammerhead” type of ribozymes, it is also preferred that the target sequences of the ribozymes be chosen from the unique regions. Ribozymes are RNA molecules which possess highly specific endoribonuclease activity. Hammerhead ribozymes comprise a hybridizing region which is complementary in nucleotide sequence to at least part of the target RNA, and a catalytic region which is adapted to cleave the target RNA. The hybridizing region contains nine or more nucleotides. Therefore, the hammerhead ribozymes of the present invention have a hybridizing region which is complementary to the sequences listed above and is at least nine nucleotides in length. The construction and production of such ribozymes is well known in the art and is described more fully in Haseloff J. and Gerlach W. L., 1988, Nature, 334: 585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug et al., 1984, Science, 224: 574-578; Zaug A. J. and Cech T. R, 1986, Science, 231: 470-475; Zaug, et al., 1986, Nature, 324: 429-433; published International patent application No. WO 88/04300 by University Patents Inc. June, 1988; Been M. D. and Cech T. R, 1986, Cell, 47: 207-216). The Cech endoribonucleases have an eight base pair active site which hybridizes to a target RNA sequence whereafter cleavage of the target RNA takes place. The invention encompasses those Cech-type ribozymes which target eight base-pair active site sequences that are present in a subject polynucleotide but not other polynucleotides for fatty acid enzymes.

The compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense, ribozyme, triple helix, or subject polynucleotide molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195: 115-123; Hanahan et al., 1983, J. Mol. Biol., 166: 557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

Composition, Formulation, and Administration of Pharmaceutical Compositions

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, the active agent may be administered to an individual as an injectable composition, for example as a sterile aqueous dispersion, preferably isotonic. A therapeutically effective dose further refers to that amount of the compound sufficient to result in amelioration of symptoms associated with such disorders. Techniques for formulation and administration of the compounds of the instant application may be found in Mack E. W., 1990, Remington's Pharmaceutical Sciences, Mack Publishing Company, Easton, Pa., 13^{th} edition. For administration to mammals, and particularly humans, it is expected that the daily dosage level of the active agent will be from 0.001 mg/kg to 10 mg/kg, typically around 0.01 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

Thus, the present invention provides a method for screening and selecting compounds, which promote lipid metabolism disorders, and a method for screening and selecting compounds, which treat or inhibit lipid metabolism disorders, as well as diabetic neuropathy. The selected antagonists and agonists may be administered, for instance, to inhibit progressive and acute disorders, such as arterial hypertension, hypercholesterolemia, atherosclerotic heart disease, chronic inflammatory and autoimmune disorders, allergic eczema and other atopic disorders, and cancers, including human pancreatic cancer.

Antagonists, agonists and other compounds of the present invention may be employed alone or in conjunction with other compounds, such as therapeutic compounds. The pharmaceutical compositions may be administered in any effective, convenient manner including, for instance, administration by direct microinjection into the affected area or by intravenous or other routes. These compositions of the present invention may be employed in combination with a non-sterile or sterile carrier or carriers for use with cells, tissues or organisms, such as a pharmaceutical carrier suitable for administration to a subject. Such compositions comprise, for instance, a medium additive or a therapeutically effective amount of antagonists or agonists of the invention and a pharmaceutically acceptable carrier or excipient. Such carriers may include, but are not limited to, saline, buffered saline, dextrose, water, glycerol, ethanol and combinations thereof. The formulation is prepared to suit the mode of administration.

The invention further provides diagnostic and pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, the active agent may be administered to an individual as an injectable composition, for example as a sterile aqueous dispersion, preferably isotonic. For administration to mammals, and particularly humans, it is expected that the daily dosage level of the active agent will be from 0.001 mg/kg to 10 mg/kg, typically around 0.01 mg/kg. The physician in any event will determine the actual dosage that will be most suitable for an individual and will vary with the age, weight and response of the particular individual. The above dosages are exemplary of the average case. There can, of course, be individual instances where higher or lower dosage ranges are merited, and such are within the scope of this invention.

It is understood that the present invention is not limited to the particular methodology, protocols, cell lines, vectors, and reagents described herein. Generally, the laboratory procedures in cell culture and molecular genetics described below are those well known and commonly employed in the art. Standard techniques are used for recombinant nucleic acid methods, polynucleotide synthesis, microbial culture, transformation, transfection, etc. Generally, enzymatic reactions and purification steps are performed according to the manufacturer's specifications. Although any methods and materials similar or equivalent to those described herein can be used in the practice or testing of the present invention, the selected methods, devices, and materials are described below.

### EXAMPLES

### Example 1

### Human Diabetic Clinical Study

In order to determine the relationship between lipid profiles and the expression of lipid metabolic genes in insulin-dependent (IDDM, Type 1) and non-insulin dependent (NIDDM, Type 2) diabetics, a clinical study was conducted with diabetic patients and with an age-matched control population.

The study examined the lipid profiles in about eighty insulin dependent and non-insulin dependent diabetics. Both types of diabetes are associated with impaired fatty acid metabolism. The data shown below in the following tables were obtained in a clinical study completed at QuantaNova Canada Ltd. The data indicate that there are significant differences in the fatty acid profiles of red blood cell phospholipids and plasma phospholipids between diabetics and the controls.

The changes observed in the serum chemistry data were consistent with other published studies. Table 2 shows the increase in triglyceride and subsequent decrease in the HDL levels in the Type 2 diabetic patients, which has been reported previously by Persson et al., 1996, Scand. J. Clin. Lab. Invest., 56: 183-190; Betteridge D. J., 1999, Eur. J. Clin. Invest., Vol. June 29, Suppl. 2: 12-6, and Kreisberg R. A., 1998, Am. J. Cardiol., 82: 67U-73U discussion 85U-86U. The cholesterol and LDL levels were reduced in the diabetic groups compared to the control group.

TABLE 2
Serum Chemistry Parameters

| Clinical | | | |
|---|---|---|---|
| Measurement | Type 1 Diabetic | Type 2 Diabetic | |
| (mmol/l) | Patients | Patients | Control Patients |
| Triglyceride | 1.85 ± 1.19 | 2.51 ± 1.39** | 1.89 ± 1.36 |
| Cholesterol | 4.28 ± 0.73 | 4.51 ± 0.82 | 4.91 ± 0.92** |
| HDL | 0.96 ± 0.28 | 0.92 ± 0.31* | 1.04 ± 0.30 |
| Cholesterol | | | |
| LDL | 2.48 ± 0.68 | 2.51 ± 0.85 | 2.99 ± 0.81** |
| Cholesterol | | | |
| Glucose | 7.99 ± 4.79 | 7.66 ± 2.02 | 4.82 ± 0.61** |

*indicates statistically significant difference from the control group, p < 0.05 two tailed t-test using unequal variances
**indicates statistically significant difference from the other two groups, p < 0.05 two tailed t-test using unequal variances

Table 3 shows that the amount of linoleic acid is increased in the red blood cell phospholipids of Type 1 diabetics. Arisaka et al., 1986, J. Paediatr. Gastroenterol. Nutr., 5: 878-882, Tilvis R. S. and Miettinen T. A., 1985, J. Clin. Endocrinol. Metab., 61: 741-745, and Van Doormaal et al., 1988, Diabetologia, 31: 576-584 have reported that insulin-dependent diabetics have an increased concentration of linoleic acid in their plasma. They were unable to show the same increase in linoleic acid in red blood cells. However, with large sample sizes and improved analysis techniques we have on two separate occasions demonstrated that the amount of linoleic acid does indeed increase in the red cells of Type 1 diabetics. The higher concentration of linoleic acid present in the diabetic samples suggests that the provision of essential fatty acid precursors in the diet is adequate in the diabetic population and any changes in longer chain fatty acids were most likely due to alterations in the activity of the lipid metabolic enzymes and/or their genes.

TABLE 3
Linoleic Acid Concentrations in RBC Total Phospholipids (PL) and
in two PL Sub-fractions: Phosphatidylethanolamine (PE) and
Phosphatidylcholine (PC) (mg/100 mg fatty acid)

| | Type 1 Diabetic | Type 2 Diabetic | |
|---|---|---|---|
| PL Fraction | Patients | Patients | Control Patients |
| Total PL | 11.77 ± 1.98* | 10.81 ± 1.54 | 11.00 ± 1.30 |
| PE | 5.46 ± 0.97 | 4.90 ± 1.01 | 5.18 ± 1.10 |
| PC | 19.90 ± 2.04** | 18.56 ± 2.88 | 18.23 ± 2.84 |

*indicates statistically significant difference from the Type 2 group, p < 0.05 two tailed t-test using unequal variances
**indicates statistically significant difference from the other two groups, p < 0.05 two tailed t-test using unequal variances

Table 4 shows the concentrations of six fatty acids in the plasma phospholipid fraction. The linoleic acid concentration in the plasma phospholipids showed the same profile as in the red blood cell phospholipids. The concentration of linoleic acid was increased in the Type 1 diabetic group compared to the Type 2 and control groups. Of particular importance was the decrease in the arachidonic acid concentration in the Type 1 diabetics. This provides further evidence that the n-6 metabolic pathway, which converts linoleic acid to arachidonic acid, is impaired in Type 1 diabetics. The concentration of long chain n-3 polyunsaturated fatty acids (i.e. eicosapentaenoic and docosahexaenoic acids) was decreased in the Type 1 diabetic group compared to the Type 2 and control groups.

TABLE 4
Fatty Acid Concentrations in Plasma PL (mg/100 mg fatty acid)

| | Type 1 Diabetic | Type 2 Diabetic | Control |
|---|---|---|---|
| Fatty Acid | Patients | Patients | Patients |
| Oleic acid (OA) | 10.57 ± 1.76* | 9.75 ± 1.19 | 9.97 ± 1.39 |
| Linoleic Acid | 24.80 ± 3.39* | 22.77 ± 3.12 | 23.45 ± 3.27 |
| (LA) | | | |
| Arachidonic Acid | 12.19 ± 1.95* | 13.18 ± 2.18 | 13.20 ± 2.39 |
| (AA) | | | |
| Dihomogamma- | 3.35 ± 0.88 | 3.70 ± 0.74*** | 3.30 ± 0.77 |
| Linolenic Acid | | | |
| (DGLA) | | | |
| Eicosapentaenoic | 1.03 ± 0.41** | 1.68 ± 0.86 | 1.58 ± 0.97 |
| Acid (EPA) | | | |
| Docosahexaenoic | 3.57 ± 1.04* | 4.21 ± 0.93 | 3.90 ± 2.39 |
| Acid (DHA) | | | |

*indicates statistically significant difference from the Type 2 group, p < 0.05 two tailed t-test using unequal variances
**indicates statistically significant difference from the other two groups, p < 0.05 two tailed t-test using unequal variances
***indicates statistically significant difference from the control group, p < 0.05 two tailed t-test using unequal variances

Accordingly, the above clinical data clearly show that lipid metabolism is altered in diabetic individuals in a way that cannot be accounted for by a deficiency in the precursor dietary fatty acids. When these data are combined with data derived from rat models of diabetes (see Examples 2 and 3), evidence points to the substantial role played by the fatty acid desaturases in altering the fatty acid profiles of diabetics.

Materials and Methods

Subjects: 34 Type 1 diabetics, 47 Type 2 diabetics, 44 Controls

Sample Collection and Preparation:

Blood was collected from fasted diabetic and control patients via venous puncture using 10 ml Vacutainers containing EDTA as an anticoagulant The whole blood was centrifuged at 4° C. for 15 min at 2500 rpm. The resulting plasma was transferred to a labelled tube and immediately stored at −20° C. The red cells were washed with an equal volume of saline. The saline was added to the tube and then mixed before centrifugation as described above. The upper layer was discarded. This procedure for washing red cells was repeated twice.

Sample Extractions

Plasma:

A known amount of standard was added to each millilitre of plasma before the extraction process. Total lipids were extracted from the plasma using chloroform: methanol (2:1, v/v) according to the method of Folch et al., 1957, J. Biol Chem., 226: 497-509.

Red Blood Cells:

A known amount of standard was added to 2 ml of a 1:1 (v/v) mix of RBCs and water. Ten ml of methanol was added to the mixture. The mixture was vortexed and allowed to equilibrate for 30 min. Twenty ml of chloroform was added to the mixture. After vortexing, the mixture was filtered through Whatman #1 filter paper. The filtrate had 5 ml of 0.9% saline added and was vortexed. After centrifugation for 10 minutes at 1500 rpm the top layer was removed by vacuum pump and the bottom layer was transferred to a new tube. The lipid extract was dried under nitrogen to remove the solvent The lipid extract was redissolved in 100 μl chloroform and stored at −20° C.

Thin-Layer Chromatography:

The different lipid classes from the RBCs and plasma were separated using neutral lipid thin-layer chromatography. Two samples were run on a 20 cm ×20 cm 250 um silica gel 60F plate. The solvent system used was hexane: diethyl ether: acetic acid (80:20:1 v/v). Once the plate was fully developed it was sprayed with 2,7-dichlorofluorescein to indicate the lipid class fractions. The silica gel containing each of the lipid class bands was scraped from the plate and placed in a 16×125 mm screw cap test tube.

Sample Saponification:

Adding 4 ml HPLC methanol and 0.4 ml potassium hydroxide to the scraped TLC fraction saponified the plasma cholesterol ester fraction. This mixture was heated for 1 hr at 90° C. The saponified extract was extracted using 2 ml 0.9% saline and 5 ml HPLC hexane. The mixture was vortexed and the organic layer discarded The remaining aqueous phase was acidified with 0.4 ml concentrated hydrochloric acid. Five ml of HPLC hexane was added and the mixture vortexed The organic layer was transferred to another tube and dried under nitrogen to remove the excess hexane.

Sample Methylation:

All sample fractions were methylated by adding BF₃-methanol and heating to 90° C. for 30 minutes according to the method of Folch et al., 1957, J. Biol. Chem., 226: 497-509. The resultant fatty acid methyl esters (FAME) were analyzed on a gas chromatograph.

Gas Chromatograph Parameters:

The FAME profile was determined using a Hewlett Packard Gas Chromatograph equipped with an interfaced ChemStation, a flame-ionization detector and a 30 m×0.25 mm i.d. fused silica column (HP-wax, cross linked polyethylene glycol, film thickness 0.25 μm) and He as gas carrier. The temperatures of the injector and detector were maintained at 225° C. and 250° C., respectively. After an initial hold of 1 min at 180° C., the column temperature was increased by 4° C./min to 190° C. (7 min hold), then by 10° C./min to 200° C. (5 min hold) and finally by 25° C./min to 215° C. This temperature was maintained for 17.9 min. FAME were identified by comparison with authentic standards.

### Example 2

### Streptozotocin-Induced Diabetic Rat Study

This study was designed to compare and correlate changes in the concentrations of tissue fatty acids to the activity of fatty acid delta-5 and delta-6-desaturases.

Rat Liver Fatty Acid Profiles

This part of the study was designed to compare the changes in tissue fatty acid profiles from different lipid classes between streptozotocin induced diabetic rats and control rats. For the purpose of this report all changes in fatty acid levels reported are significant to p<0.01.

Table 5 contains fatty acid data from the phospholipid fraction in rat liver. Although data from the other main lipid classes (i.e. triglycerides, cholesterol esters and free fatty acids) show substantially similar trends, only the phospholipid data is presented herewith. These data help to demonstrate the activities within the n-6 and n-3 fatty acid metabolic pathways. The relative amount of linoleic acid (LA) increased in both the 2 and 7 week diabetic groups (20.42±1.29 and 16.67±1.44 mg/100 mg fatty acid respectively) compared to their respective control groups (11.99±0.73 and 11.99±0.93 mg/100 mg fatty acid respectively). The LA level was also decreased in the 7 week diabetic rats compared to the 2 week diabetic rats. The level of dihomogamma-linolenic acid (DGLA) was unchanged among the experimental groups. The arachidonic acid (AA) level was decreased in both diabetic groups compared to the control groups. The level of AA was reduced from 26.90±0.48 and 23.70±1.68 mg/100 mg fatty acid in the 2 and 7 week control groups to 18.53±1.84 and 17.40±2.45 mg/100 mg fatty acid in the 2 and 7 week diabetic groups. It was also noted that the level of AA was decreased in the 7 week control group versus the 2 week control group.

TABLE 5
Liver Phospholipid Fatty Acid Levels (mg/100 mg fatty acid)

| | Control 2 Week | Diabetic 2 Week | Control 7 Week | Diabetic 7 Week |
|---|---|---|---|---|
| Fatty Acid | (n = 6) | (n = 6) | (n = 6) | (n = 9) |
| n-6 | | | | |
| LA | 11.99 ± 0.73 | 20.42 ± 1.29^{a} | 11.99 ± 0.93^{b,c} | 16.67 ± 1.44 |
| DGLA | 0.96 ± 0.08 | 0.96 ± 0.07 | 1.22 ± 0.19 | 1.14 ± 0.29 |
| AA | 26.90 ± 0.48 | 18.53 ± 1.84^{a} | 23.70 ± 1.68^{b} | 17.40 ± 2.45^{d} |
| n-3 | | | | |
| EPA | 1.79 ± 0.50 | 1.26 ± 0.23 | 1.81 ± 0.19 | 0.71 ± 0.30^{b,c} |
| DPA | 0.97 ± 0.05 | 1.43 ± 0.28^{a} | 1.11 ± 0.21 | 1.28 ± 0.23 |
| DHA | 15.13 ± 2.14 | 15.36 ± 1.53 | 13.58 ± 1.55 | 18.14 ± 1.79^{b,c} |
| Saturates | | | | |
| 16:0 | 12.68 ± 0.46 | 15.15 ± 0.75^{a} | 13.30 ± 0.88 | 15.92 ± 0.93^{b} |
| 18:0 | 22.24 ± 1.52 | 19.53 ± 2.04 | 24.01 ± 1.83 | 21.12 ± 1.76 |
| Monoenoic | | | | |
| 16:1n-7 | 0.40 ± 0.05 | 0.22 ± 0.03^{a} | 0.57 ± 0.13 | 0.20 ± 0.11^{b} |
| 18:1n-7 | 1.82 ± 0.19 | 1.69 ± 0.14 | 1.84 ± 0.24 | 1.44 ± 0.17^{b,c} |
| 18:1n-9 | 2.17 ± 0.34 | 2.55 ± 0.29 | 3.24 ± 0.49^{d} | 3.18 ± 0.69 |
| Liver phospholipid fatty acid profiles. Normal control rats (sham injected) and streptozotocin induced diabetic rats were | | | | |
| # sacrificed at 2 weeks and 7 weeks after the onset of diabetes. The fatty acids are expressed as means SD (mg/100 mg fatty acid). | | | | |

^{a}2 week diabetic group vs 2 week control group, p < 0.01
^{b}7 week diabetic group vs 7 week control group, p < 0.01
^{c}7 week diabetic group vs 2 week diabetic group, p < 0.01
^{d}7 week control group vs 2 week control group, p < 0.01

The relative amount of eicosapentaenoic acid (EPA) was decreased in the 7 week diabetic group compared to the 7 week control and 2 week diabetic groups. There was an increase in docosapentaenoic acid (DPA) in the 2 week diabetic group compared to the 2 week control group. The level of docosahexaenoic acid was increased from 13.58±1.55 in the 7 week control group and 15.36±1.53 in the 2 week diabetic group to 18.14±1.79 mg/100 mg fatty acid in the 7 week diabetic group.

The liver phospholipid fraction had increased palmitic acid in both diabetic groups compared to their respective controls.

Monounsaturated fatty acid profiles of the experimental groups show that the level of palmitoleic acid (16:1n-7) was reduced in the diabetic groups compared to the control groups. The elongation product of palmitoleic acid, vaccenic acid (18:1n-7), was decreased in the 7 week diabetic group compared to the 7 week control and 2 week diabetic groups. The oleic acid (18:1n-9) level was increased in the 7 week control group (3.24±0.49 mg/100 mg fatty acid) compared to the 2 week control group (2.17±0.34 mg/100 mg fatty acid).

The increase in LA and decrease in AA concentrations in the liver phospholipid fraction is indicative of the diabetic condition in rats and is well documented in the literature. Mimouni and Poisson (1991) demonstrated these same changes in fatty acid profiles from liver phospholipids in Wistar Bio-Breeding (BB) diabetic rats (Mimouni V. and Poisson J. P., 1991, Archives internationales de Physiologie et de Biochimie, 99: 111-121). Arisaka et al., 1986, J. Paediatr. Gastroenterol Nutr., 5: 878-882 and Van Doormaal et al., 1988, Diabetologia, 31: 576-584 showed that LA was increased in the plasma phospholipid fraction of human Type 1 diabetics. Recently, applicant's own human clinical diabetic study (refer to Example 1) provided data indicating that AA was reduced in the plasma and red blood cell phospholipids of Type 1 diabetic patients. Shin et al., 1995, Diabetes Research and Clinical Practice, 29: 93-98 have reported that the levels of LA were increased and the AA content was decreased in the membranes of liver microsomes from diabetic rats. These fatty acid changes are in part, due to the result of decreased delta-6 and delta-5-desaturase activities in the tissues of diabetic humans and rats (refer to Examples 1 and 3). Reduced activity of the desaturase system in diabetics was first reported by Brenner et al., 1968, Am J. Physiol., 215: 63-70. Subsequently, this finding has been verified by Mimouni V. and Poisson J. P., 1992, Biochim. Biophys. Acta, 1123: 296-302, Dang et al., 1989, Lipids, 24: 882-889, and Faas F. H. and Carter, 1980, Lipids, 15: 953-961 and is considered to be a key factor in the development of secondary complications of diabetes.

With reduced activities of the desaturase enyzme system indicated, the changes in n-3 fatty acids should parallel the changes observed in the n-6 pathway. The EPA concentration does decrease after 7 weeks, however, DHA levels in the phospholipids increase after 7 weeks. Faas F. H. and Carter W. J., 1983, Lipids, 18: 339-342 observed similar increases in DHA in the liver phosphatidylcholine and phosphatidylethanolamine (the two major sub-classes of phospholipids) total lipid fractions of STZ diabetic rats. Giron et al., 1999, Metabolism, 48: 455-460 found higher levels of DHA in the liver of STZ induced diabetic rats.

The increased levels of palmitic acid in the diabetic rats with concomitant decreases in palmitoleic acid suggest a reduced activity of the delta-9-desaturase. The reduced activity of delta-9-desaturase has been confirmed by the direct measurement of RNA levels and enzyme activity. As mentioned previously, the activities of desaturase systems are reduced in diabetes. Dang et al., 1989, Lipids, 24: 882-889 observed that insulin treatment increased the activity of delta-6-desaturase and super-induces the delta-9-desaturase. The decrease of the vaccenic acid (18:1n-7) in the diabetic rats suggests that the elongation step from palmitoleic to vaccenic acid has been affected as well. Kawashima Y. and Kozuka H., 1985, Biochimica et Biophysica Acta, 834: 118-12 showed that diabetic rats had reduced hepatic microsomal fatty acid chain elongation activity, which could be reversed by insulin therapy.

It is well accepted that delta-6 and delta-5-desaturase activities are reduced with age. Biagi et al., 1991, Biochimica et Biophysica Acta, 1083: 187-192 and Ulman et al., 1991, Lipids: 26: 127-133 both demonstrated that delta-6-desaturase was reduced in older rats and by administering gamma-linolenic acid the activity could be partially restored. As a result of aging, there appears to be a decrease in the long chain polyunsaturated fatty acids and increase in the saturated and monoenoic acids in tissue membranes. This same phenomenon was evident in this current study. In the 7 week control group there was a 12% decrease in phospholipid AA content while oleic acid levels increased by 49%. The effect of aging is a factor, which needs to be addressed, when interpreting results from long term animal studies.

In conclusion, the fatty acid data presented herein support the findings of other researchers that lipid metabolism is altered in STZ induced diabetic rats. When these data are combined with the gene expression and enzyme activity data, it is possible to identify specific enzymatic steps that are affected in diabetes.

Materials and Methods

Animals:

Thirty female Wistar rats were received from Charles River, St-Constant, Quebec, Canada on Oct. 6, 1999. The animals were ca. 8-10 weeks of age and in the weight range of 209-246 g.

The rats were randomly divided into 1 group of 18 rats and one group of 12 rats.

Animal Husbandry:

Rats were identified by numbers tattooed on their tails and were housed in barrier maintained animal rooms at 22±2° C. and a target relative humidity of 50±10% with 15 air exchanges per hour. A 12 h light/dark cycle was controlled by a time switch, light hours being 0600-1800 h. Four rats were housed in each suspended polycarbonate cage (59×39×20 cm) with stainless steel wire grid tops. After the administration of the streptozotocin, the rats that were treated were housed 2 per cage. Wood shavings were used as bedding material. Each cage was supplied with at least one 500 ml polycarbonate water bottle with a stainless steel sipper cap. The water and feed were supplied ad libitum. All animals were monitored daily according to standard procedures.

Materials and Doses:

Streptozotocin (STZ): 2-deoxy-2-[([methylnitroso-amino]carbonyl)amino]-D-glucopyranose (Sigma S-0130, St Louis, Mo.)

Regular Chow: Product No. 8729C Teklad Certified Rodent Diet

18 rats injected with 75 mg streptozotocin/kg bd wt

| | |
|---|---|
| Group 1 | 2 week regular chow control rats |
| Group 2 | 2 week STZ diabetic rats |
| Group 3 | 7 week regular chow control rats |
| Group 4 | 7 week STZ diabetic rats |

Experimental Procedure:

Eighteen rats were injected with 75 mg STZ/kg bd wt to induce the diabetic state. After 4 days the blood glucose level in each of the STZ rats was checked. The blood sample was obtained from the tail vein. Rats, which did not have a blood glucose level of 16.7 mmol/l (>300 mg/dl) were rejected from the study. The diabetic rats were housed 2 per cage. After 2 weeks, approximately one half of the diabetic rats and control rats were sacrificed. After 7 weeks, the remaining diabetic and control rats were sacrificed. By sacrificing controls at the same time we eliminated any possible age effect when completing our data analysis. The rats were sacrificed by exsanguination and tissues were removed for fatty acid analyses.

Sample Preparation:

Blood was collected into a 10 ml syringe containing 200 ml of a 5% solution of EDTA The whole blood was centriged at 4° C. for 15 min at 2500 rpm. The plasma was transferred to a labelled tube and immediately stored at −20° C. The RBC fraction was washed with an equal volume of saline and then centrifuged at 4° C. for 15 min at 2500 rpm. The RBCs were washed twice and then stored at −20° C.

Liver Extractions:

Ten ml chloroform:methanol (2:1) was added to a slice of liver (approximately 0.5 g) and ground with a Polytron homogenizer for 30 sec. This homogenate was transferred to a conical tube with the addition of another 10 ml chloroform:methanol (2:1). Four ml 0.9% saline was added. The mixture was vortexed and allowed to stand at −4° C. After centrifugation for 10 min at 1500 rpm the organic phase was dried under nitrogen. The lipid extract was re-dissolved in 1 ml chloroform and stored at −20° C.

Thin-Layer Chromatography:

The different lipid classes from the RBCs (phospholipids), plasma and liver (phospholipids, free fatty acids, triglycerides and cholesterol esters) were separated using neutral lipid thin-layer chromatography. The samples were run on a 20 cm ×20 cm, 250 mm silica gel 60F plate. The solvent was hexane:diethylether:acetic acid (80:20:1, v:v:v). Compounds were detected by spraying the plates with 2,7-dichlorofluorescein and the silica gel containing each of the lipid class bands was scraped from the plate and placed in screw cap test tubes.

Saponification:

The plasma cholesterol ester fraction was saponified by adding 4 ml HPLC methanol and 0.4 ml potassium hydroxide. This mixture was heated for 1 hr at 90° C. The saponified extract was extracted using 2 ml 0.9% saline and 5 ml HPLC hexane. The mixture was vortexed and the remaining aqueous phase was acidified with 0.4 ml concentrated hydrochloric acid. Five ml of HPLC hexane was added and the mixture vortexed. The organic layer was dried under nitrogen.

Methylation:

Four ml of BF₃-methanol was added to the scraped silica gel fractions or the dry cholesterol ester fraction and heated for 30 min at 90° C. The extracts were then cooled and extracted once again with 2.0 ml 0.9% saline and 5.0 ml HPLC grade hexane. The samples were vortexed for 30 sec and then centrifuged at 1500 rpm for 2 min. The top hexane layer was dried under nitrogen. The fatty acid methyl esters (FAME) were dissolved in HPLC grade hexane and analyzed by gas chromatography.

Gas Chromatograph Parameters:

The FAME profile was determined using a Hewlett Packard Gas Chromatograph equipped with an interfaced ChemStation, a flame-ionization detector and a 30 m×0.25 mm I.D. fused silica column (HP-wax, cross linked polyethylene glycol, film thickness 0.25 μm) and He as gas carrier. The temperatures of the injector and detector were maintained at 225° C. and 250° C., respectively. After an initial hold of 1 min at 180° C., the column temperature was increased by 4° C./min to 190° C. (7 min hold), then by 10° C./min to 200° C. (5 min hold) and finally by 25° C./min to 215° C. This temperature was maintained for 17.9 min. FAME were identified by comparison with authentic standards.

Statistical Analysis:

The differences in the group fatty acid level means were statistically analysed using the Student's T-test with a two-tailed test at p<0.01 and unequal variances.

### EXAMPLE 3

### Rat Liver Delta-6 and Delta-5Desaturase Activity in Experimental Diabetes

This part of the study was designed to compare the activities of fatty acid desaturases between streptozotocin-induced diabetic rats and controls.

All statistical comparisons were made between treated and control animals sacrificed at the same time point since age could be a factor that affects the fatty acid desaturase activities (Hrelia et al., 1989, Biochem. Biophys. Res. Comm., 163: 348-355).

The fatty acid delta-5 and delta-6desaturase activity (expressed in pmol per mg of microsomal protein per min) were significantly reduced by approximately 37% and 28%, respectively, in hyperglycaemic animals sacrificed 2 and 7 weeks after the onset of diabetes (Table 6).

TABLE 6
Hepatic fatty acid desaturase activity in
n rmal and streptozotocin treated rats.

| | |
|---|---|
| Time after | Activity (pmol/mg |
| STZ Treatment | microsomal protein/min) |

| Rat | (weeks) | D6D | D5D |
|---|---|---|---|
| Control | 2 | 172.9 ± 21 | 290.6 ± 40 |
| STZ | 2 | 125.4 ± 34^{a} | 195.6 ± 56^{b} |
| Control | 7 | 143.1 ± 31 | 220.1 ± 30 |
| STZ | 7 | 102.8 ± 29^{c} | 129.7 ± 30^{d} |

Values are the means S.D. of at least 6 animals
^{a}and ^{b}indicate p = 0.02 and p = 0.009, respectively, when compared to Control rats at 2 weeks.
^{c}and ^{d}indicate p = 0.03 and p < 0.01, respectively, when compared to Control rats at 7 weeks.
STZ: streptozotocin treated rats; D6D and D5D: delta-6 and delta-5-desaturases, respectively.

The desaturations of either linoleic or dihomogamma-linolenic acids are reduced in hyperglycaemic rats regardless of the time after streptozotocin treatment. The fatty acid profile in hepatic phospholipids (refer to Example 1) with the exception of the docosahexaenoic acid levels reflected the delta-5 and delta-6-desaturase altered activities. Also, data on Northern blots from hepatic RNA (not shown) suggest that the decreases observed in delta-5 and delta-6desaturase activities is a reflection of RNA levels and, therefore, point to altered transcriptional and/or other pretranslational controls.

The results from the present study are consistent with those obtained in different laboratories using this and other experimental models of diabetes (Mimouni V. and Poisson J. P., 1992, Biochim. Biophys. Acta, 1123: 296-302; Faas F. H. and Carter W. J., 1980, Lipids, 15: 953-961; and Brenner et al., 1968, Am. J Physiol., 215: 63-70). These findings support the hypotheses that fatty acid desaturases are potential drug targets in diabetes and are also useful lipid metabolic compounds for drug screening assays.

Materials and Methods

Chemicals and Radiochemicals:

All organic solvents and chemicals were of reagent grade and obtained from Fisher-Scientific (Fair Lawn, N.J., U.S.A.). Lipid standards, niacinamide, N-acetylcysteine, ATP, coenzyme A and NADH were obtained from Sigma-Aldrich Canada (Oakville, ON, Canada). [1-¹⁴C]Linoleic and [1-¹⁴C]dihomo-gamma-linolenic acids (99% radiochemical purity; 51 and 52 mCi/mmol, respectively) were purchased from DuPont Canada Inc. (Markham, Ontario, Canada).

Isolation of Hepatic Microsomes:

As described in other Examples, female Wistar rats were intraperitoneally (i.p.) injected with 50 mg of streptozotocin per kg of body weight. Three days later, animals received a second dose of streptozotocin (25 mg/kg body weight). Two and 7 weeks after the onset of diabetes, non-fasted control and streptozotocin (blood glucose levels 21 to >33 mmoles/L) treated rats were put under light halothane (15% in mineral oil) anaesthesia and sacrificed by exsanguination between 9:00 and 10:00 a.m. Under these experimental conditions, variations in enzyme activity caused by circadian rhythm (Actis Dato et al., 1973, Lipids, 8: 1-6) can be avoided and a substantial (although submaximal) activity of liver desaturase can be obtained (Actis Dato et al., 1973, Lipids, 8: 1-6 and Inkpen et al., 1969, J. Lipid Res., 10: 277-282). Livers were quickly rinsed with cold 0.9% NaCl solution, weighed and minced with scissors. All procedures were performed at 4° C. unless otherwise specified. Microsomes were isolated by differential ultracentrifugation as previously described (de Antueno et al., 1994, Lipids, 29: 327-331). Briefly, livers were homogenized in a solution (1:3 w/v) containing 0.25 M sucrose, 62 mM potassium phosphate buffer (pH 7.0), 0.15 M KCl, 1.5 mM N-acetylcysteine, 5 mM MgCl₂, and 0.1 mM EDTA using 4 strokes of a Potter-Elvehjem tissue homogenizer. The homogenate was centriged at 10,400× g for 20 min to eliminate mitochondria and cellular debris. The supernatant was filtered through a 3-layer cheesecloth and was centriged at 105,000×g for 60 min. The microsomal pellet was gently resuspended in the same homogenization solution with a small glass/teflon homogenizer and kept frozen at −70° C. until used (Leikin A. I. and Brenner R. R., 1987, Biochim. Biophys. Acta, 922: 294-303). The absence of mitochondrial contamination was enzymatically assessed as previously described (Kilberg M. S. and Christensen H. N., 1979, Biochemistry, 18: 1525-1530). The protein concentration was measured by the method of Lowry et al. with bovine serum albumin as the standard (Lowry et al., 1951, J. Biol. Chem., 193: 265-275).

Desaturase Assays:

The activities of delta-5 and delta-6 desaturases were determined by measuring the conversion of [1-¹⁴C]20:3n-6 (dihomogamma-linolenic acid) to [1-¹⁴C]20:4n-6 (arachidonic acid) and [1-¹⁴C]18:2n-6 (linoleic acid) to [1-¹⁴C]18:3n-6 (gamma-linolenic acid), respectively. In this study the standard methodology accepted in most laboratories was used.

As proposed by Leikin and Brenner (Leikin A. I. and Brenner R. R., 1989, Biochim. Biophys. Acta. September 25, 1005: 187-191), the following are best conditions for the desaturation assays since the enzymes are saturated with the substrates and the reactions are linear within the incubation time. Reactions were started by adding 2 or 3 mg of microsomal protein to pre-incubated tubes containing 0.20 μCi of the substrate fatty acid at a final concentration of 33.3 μM in 1.5 ml of the homogenization solution, containing NaF (42 mM), niacinamide (0.33 mM), ATP (1.57 mM), NADH (1.01 mM) and coenzyme A (0.09 mM) as described elsewhere (Leikin A. I. and Brenner R. R., 1989, Biochim. Biophys. Acta. September 25,1005: 187-191).The tubes were vortexed vigorously and after 15 min incubation in a shaling water bath (37° C.), the reactions were stopped by the addition of 2 ml of 10% (w/v) KOH in ethanol. Lipids in the incubation mixture were saponified at 80° C. for 45 min under N₂. The samples were then left in ice for 5 min before acidification. The fatty acids were extracted with hexane and esterified with BF₃/methanol at 90° C. for 30 min (Morrison W. R. and Smith L. M., 1964, J. Lipid Res., 5: 600-608).

Radiolabeled fatty acid methyl esters (FAME) were analyzed as previously described (de Antueno et al., 1993, Lipids, 28: 285-290). Analyses of radiolabelled FAME were carried out on a Hewlett Packard (1090, series II) chromatograph equipped with a diode array detector set at 205 nm, a radioisotope detector (model 171, Beckman, Fullerton, Calif.) with a solid scintillation cartridge (97% efficiency for ¹⁴C-detection) and a reverse-phase ODS (C-18) Beckman column (250 mm×4.6 mm i.d., 5 μm particle size) attached to a pre-column with a μBondapak C-18 (Beckmnan) insert. FAMEs were separated isocratically with acetonitrile/water (95:5, v:v) at a flow rate of 1 ml/min and were identified by comparison with authentic standards.

Statistical Analysis:

The results are expressed as means standard deviation. The significance of differences was determined using a two-tailed, Student's t-test. A difference was considered significant at P<0.01.

### Example 4

### Identification and Characterization of Human Fatty Acid Desaturase Genes

Human genomic sequences were searched via the BLAST algorithms (Altschul et al., 1990, J. Mol. Biol., 215: 403-410 and Altschul et al., 1997, Nucleic Acids Res, 25: 3389-3402) using known delta-6-desaturase sequences from Borago officinalis (GenBank Accession No. U79010) and Caenorhabditis eleganis (locus CEW08D2). BLAST, which stands for Basic Local Alignment Search Tool, produces alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin.

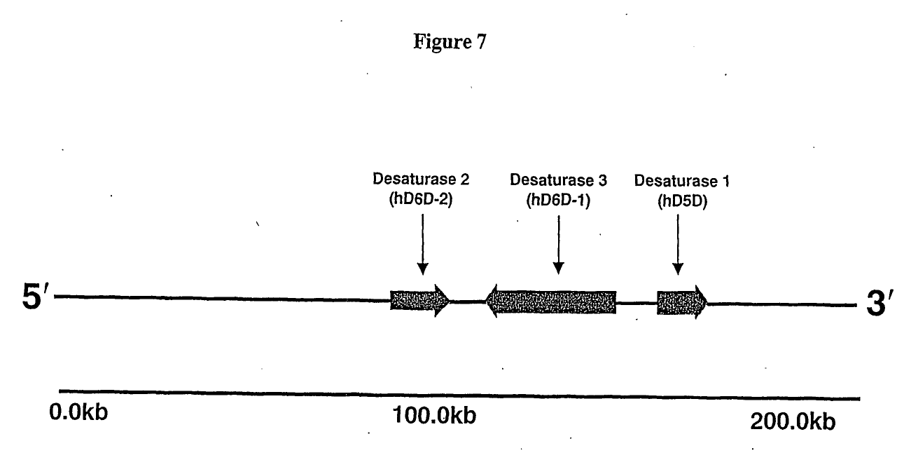

Three separate desaturase-like genes were discovered all residing side by side on a stretch of DNA (FIG. 7) contained in a bacterial artificial chromosome (BAC) assigned to chromosome 11 (GenBank Accession No. AC004770). Only two of these were annotated in the GenBank entry as putative fatty acid desaturases (hereinafter referred to as hD5D and hD6D-2).

Experimental work via RT-PCR showed that the intron/exon structure of hD5D (BC269730_{—}2) as annotated for AC004770 in GenBank was essentially correct

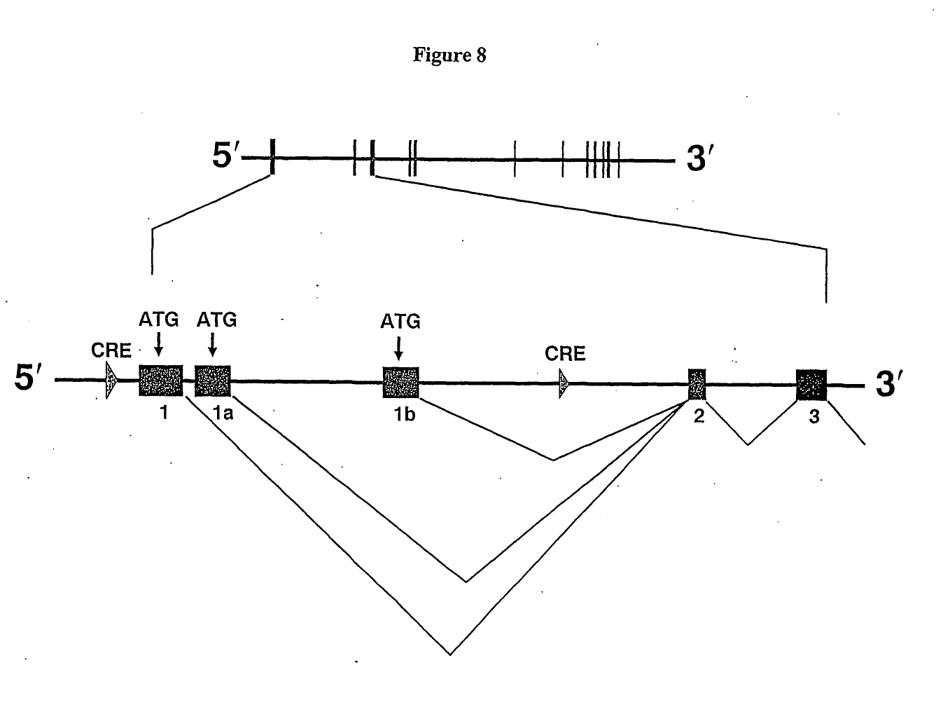

Further investigation into this desaturase using 5′-RACE (rapid amplification of cDNA ends) on human liver cDNA revealed the presence of alternate splicing for this gene. This conclusion was arrived at by DNA sequencing the 5′ end of a number of separate clones. There are at least three different exon 1s, giving rise to at least three different variants of this desaturase known as hD5D, hD5D-a and hD5Db (FIG. 8).

RT-PCR and PCR experiments using human genomic DNA with hD6D-2 (BC269730_{—}1) indicated a different 3′ splice junction for exon 8 other than that predicted by the GenBank annotation. This resulted in a frameshift of the downstream deduced amino acid sequence of this desaturase, essentially predicting a sequence with a much higher percent identity to other desaturases, including hD5D. It was also concluded that hD6D-2 was truncated at the 5′ end in this BAC clone (GenBank Accession No. AC004770). Further investigation into human genomic sequences revealed 5′ overlapping BAC clones (eg. Genank Accession No. AC004228) which contained the predicted missing exon 1 for hD6D-2. All of these predictions have been confirmed by PCR cloning and DNA sequencing.

A third desaturase, hD6D-1, was also discovered on the original BAC situated between hD5D and hD6D-2. Its exon/intron structure was confirmed via RT-PCR, PCR cloning and DNA sequencing as well, and its deduced amino acid sequence shows very high percent identity to the other two desaturase sequences.

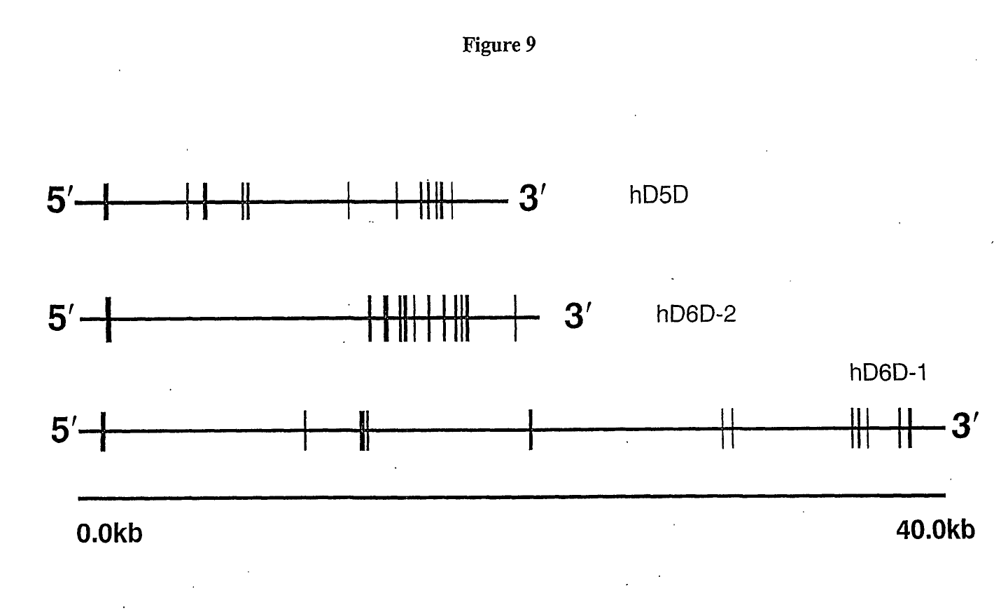

Other than the highly conserved deduced amino acid sequence of the three desaturases, they also share a conserved exon structure, having 12 exons each (FIG. 9). Clarke and co-workers have speculated that hD6D1 is a human delta-6-desaturase gene (Cho et al., 1999a, J. Biol. Chem., 274: 471-477) due to similarity to a mouse delta-6-desaturase. Applicants' work contained herein proves conclusively with functional data that hD6D-1 is, in fact, a delta-6-desaturase. Cho et al., 1999b, J. Biol. Chem., 274: 37335-37339, as well as applicants (unpublished data) have also further demonstrated that hD5D is actually a fatty acid desaturase gene encoding a human delta-5-desaturase. Moreover, hD6D-2 has been identified as a retinal specific delta-6-desaturase gene by scientists at Merck Research Laboratories, West Point, Pa., U.S.A. (GenBank Accession No. AF134404). hD6D-1 has a rat ortholog rD6D-1 (Aki et al., 1999, Biochem. Biophys. Res. Commun., 255: 575-579).

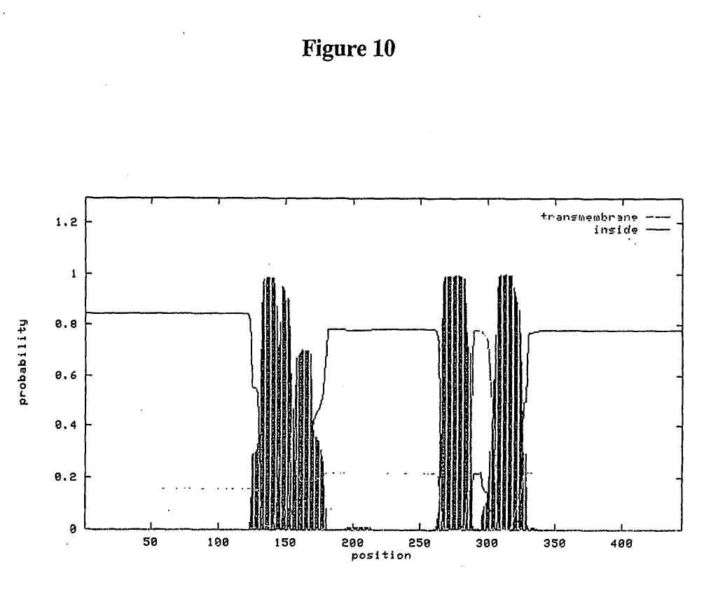

The deduced amino acid sequences of the human desaturases were submitted to the transmembrane hidden Markov model (TMHM) server at the Technical University of Denmark, Centre for Biological Sequence Analysis (Sonnhammer et al., 1998, in Proc of Sixth Int Conf on Intelligent Systems for Molecular Biology, AAAI Press, Menlo Park, Calif. pp. 175-182). Using the TMHMM algorithm, the human desaturases are predicted to have four membrane spanning domains. These are highly conserved with respect to position in the amino acid sequence. As illustrated in FIG. 10, the portion of the graph labelled inside refers to the cytosolic side of the membrane while the portion labelled outside refers to the lumen of the ER.

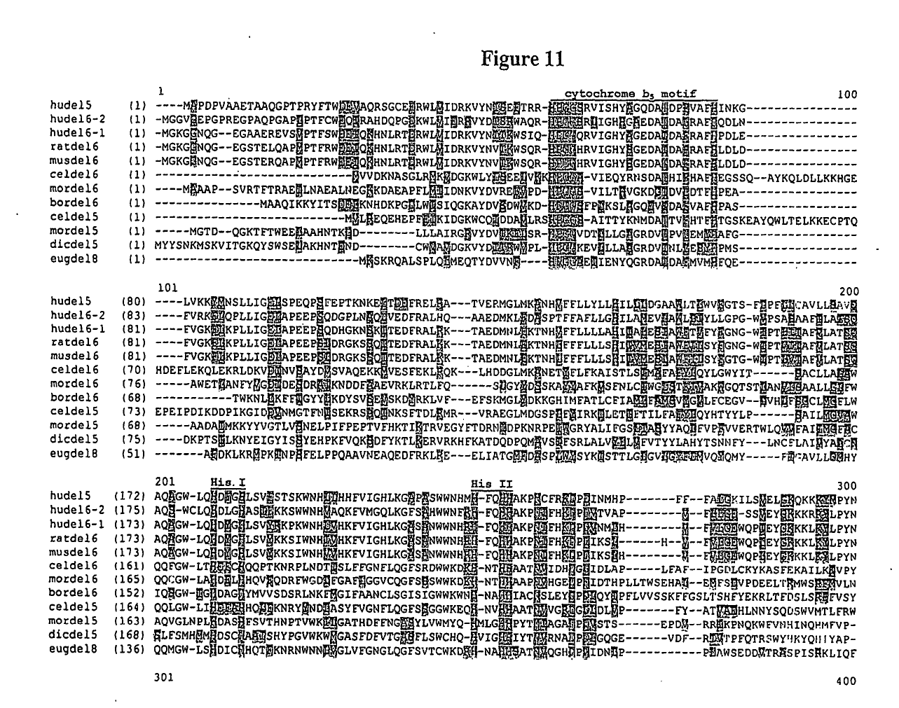

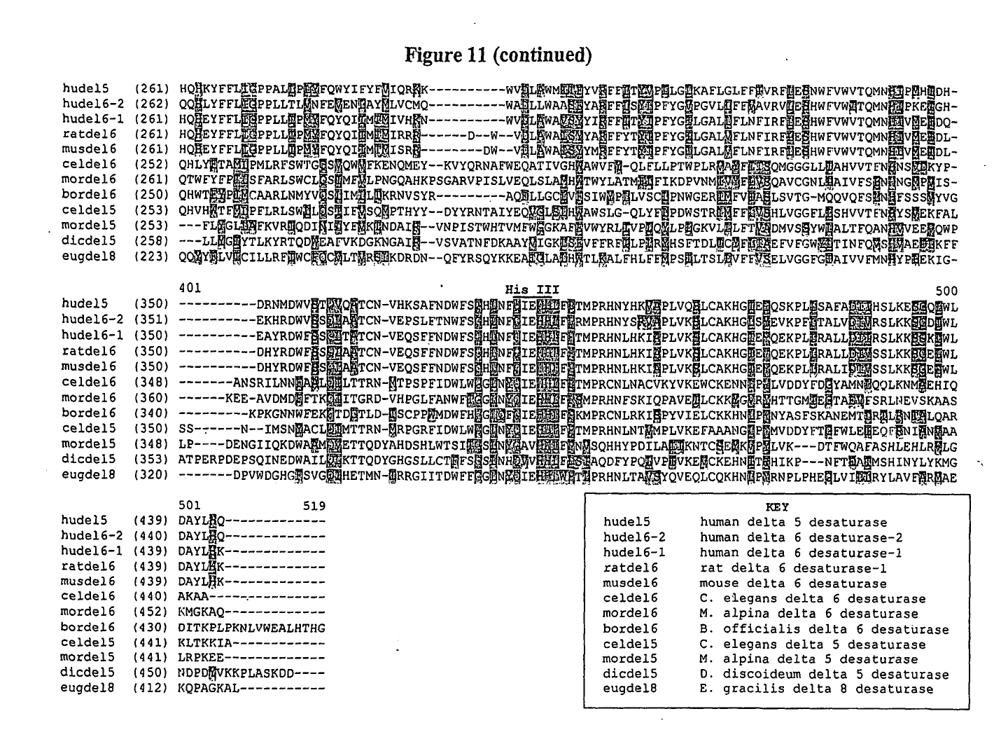

When comparing the deduced amino acid sequences of the human desaturases to other known fatty acid desaturases using a clustalw algorithm (Thompson et al., 1994, Nucleic Acid Res., 22: 4673-4680), four highly conserved regions are identified as shown in FIG. 11. One of these is the heme binding region of cytochrome b₅ and three of these are histidine boxes. The highly conserved heme binding motif in the cytochrome b₅ domain is present in hD5D, hD6D-2 and hD6D-1 but is not found in hD5D-a or hD5D-b. This is due to the fact that the amino acids encoded for by the DNA sequence for this heme binding motif span the junction between exon 1 and exon 2. They are not found in exons 1a or 1b.

All multiple alignments were performed using the clustalw algorithm in the AlignX module of Vector NTI Suite (InforMax, Inc.). The scoring matrix was blossum62 with a gap opening penalty of 10 and a gap extension penalty of 0.05.

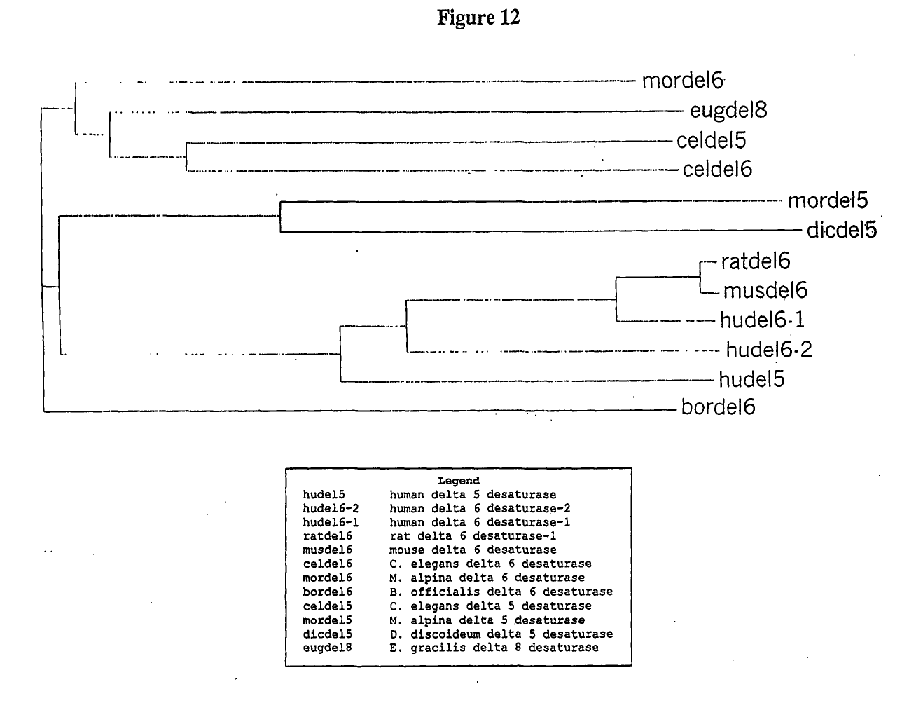

Of all of the human desaturases, hD6D-1 is most similar to other known desaturases. hD6D-1 is very similar to the rat (GenBank Accession No. BAA75496.1) and mouse (GenBank Accession No. AAD20017.1) delta-6-desaturases. Human hD5D is the most distantly related desaturase from the rat (i.e. rD6D-1) and mouse delta-6-desaturases (FIG. 12).

Dendograms were performed in AlignX and calculated based on a sequence distance method that utilizes the neighbour joining algorithm (Saitou N and Nei M., 1987, Mol. Biol. Evol., 4: 406425).

Materials and Methods

RNA Extraction:

Total RNA was extracted from the human cell line Chang (ATCC # CCL-13), using TRIzol Reagent solution (GIBCO BRL, MD) as described by the manufacturer.

Primers:

All primers for RT-PCR, genomic DNA PCR and RACE were designed using Primer Premier software (Premier Biosoft International, Palo Alto, Calif.).

Table 7 provides a list of the primers which were used in the PCR reaction for hD5D, hD6D-1 and hD6D-2.

TABLE 7
Primer Sequences for hD5D

5′-TGGGGAAGATCCTCTCTGTG-3′
5′-GGACTTGGCCTGGATGATTA-3′
5′-ACTATTGGGGCTGAAAGCCT-3′
5′-CATCGTGGGAAAAAGATGGT-3′
5′-GGGAAAAAGATGGTGCTCAA-3′
5′-ATGATCAATGTGCATGGGAA-3′

Primer Sequences for hD6D-1

5′-GGCACTACGCTGGAGAAGAT-3′
5′-CCATTCGCCCAGAACAAA-3′
5′-CCCCTGGTGATTGGTGAACT-3′
5′-AAAGGCCGTGATGAGGGTAG-3′
5′-ACACAAACCAGTGGCTCTGC-3′
5′-CCATGATCGTCCATAAGAAGT-3′
5′-ATTTGTGAAGGTAGGCGTCCAG-3′
5′-CATCCCTTTCTACGGCATCCT-3′
5′-GCGGCTTCTCCTGGTATTCA-3′
5′-GGGCCGTCAGGTACTACATC-3′
5′-TGGCTACTGAACCAGTCACG-3′

Primer Sequences for hD6D-2

5′-CCGAGGACATGAAGCTGTTTG-3′
5′-CTGCTGGTTGTAGGGTAGGTAT-3′
5′-AGTCCCACCTTCTTTGCTTTC-3′
5′-AGGGTAGGTATCTGCGTTTCTT-3′
5′-CTTCTTTGTTGCTGTCAGGGTC-3′
5′-GGTGCTCGATCTGGAAGTTGA-3′
5′-AAAATCTCTGGCGTACATGCTGG-3′
5′-AGGTGGTGCTCGATGTGGAA-3′
5′-TCAACTTCCAGATCGAGCACC-3′
5′-TCCAGCCAGATGTCACCAGA-3′
5′-GATGTGGCCCAGTAGGAAAG-3′
5′-GTTCCTACAGCCCCTGTTGA-3′

Reverse Transcription:

About 1 μg of Chang RNA was reverse-transcribed in 5 mM MgCl₂, 50 mM KCl, 20 mM Tris-HCl (pH 8.4), 2.5 μM of random hexamer primers (Perkin-Elmer, CT), 1.0 mM each dNTP, 1.0 U/μl of RNase inhibitor (Perkin-Elmer) and 2.5 U/μl of MuLV reverse transcriptase (GIBCO BRL). The reactions were carried out at 25° C. for 10 min followed by 42° C. for 15 min in a final volume of 20 μl. The enzyme was then inactivated at 99° C. for 5 min.

Polymerase Chain Reaction:

The PCR reactions were carried out in 2 mM MgCl₂, 50 mM KCl, 20 mM Tris-HCl (pH 8.4), 0.2 mM of each dNTP, 0.025 U/μl Platinum Taq (Gibco BRL). The primers were at a concentration of 0.5 μM each. After an initial denaturation at 95° C. for 2 min, the PCR reactions cycled 30 times through 95° C. for 30 sec, 60° C. for 45 sec and 72° C. for 45 sec. A final 7 min extension at 72° C. was added to the end of the cycles. The final reaction volume was 20 μl.

Rapid Amplification of cDNA Ends:

Nested 5′-RACE for hD5D was performed on Marathon-Ready™ human liver cDNA (Clontech Laboratories, Inc., Palo Alto, Calif.) as described by the manufacturer. The initial gene specific primer was 5′-CCACCCACTTCTTTCGCTGGATAACA-3′ while the nested gene specific primer was 5′-TGTGCTGGTGGTTGTACGGCATAT-3′. The PCR reactions were carried out in a Perkin-Elmer GeneAmp PCR system 9700 instrument in a 25 μl reaction volume.

The PCR cycling parameters were:

94° C. for 30 sec

5 cycles at 94° C., 30 sec followed by 74° C., 4 min

5 cycles at 94° C., 30 sec followed by 72° C., 4 min

8 cycles at 94° C., 30 sec followed by 70° C., 4 min

68° C. for 5 min

### Example 5

### Cloning of Rat and Human Desaturase Genes

RNA Extraction:

Total RNA was extracted from rat liver or the human cell line Chang (ATCC # CCL-13), using TRIzol Reagent solution (GIBCO BRL, MD) as described by the manufacturer.

Reverse Transcription:

About 1 μg of each RNA was reverse-transcribed in 3 mM MgCl₂, 75 mM KCl, 50 mM Tris-HCl (pH 8.3), 2 ng/μl of random primers (Perkin-Elmer, CT), 1.0 mM each dNTP, 2.0 U/μl of RNase inhibitor (Perkin-Elmer) and 10 U/μl of MMLV reverse transcriptase (GIBCO BRL). The reactions were carried out at 42° C. for 30 min in a final volume of 20 μl. The enzyme was inactivated at 94° C. for 5 min.

Amplification of Desaturase Genes by PCR and Cloning in Yeast Vector:

Aliquots (10 μl) of the reverse transcription reactions were amplified by polymerase chain reaction (PCR), using primers designed to generate cDNAs corresponding to the coding sequences for the rat and human desaturase genes.

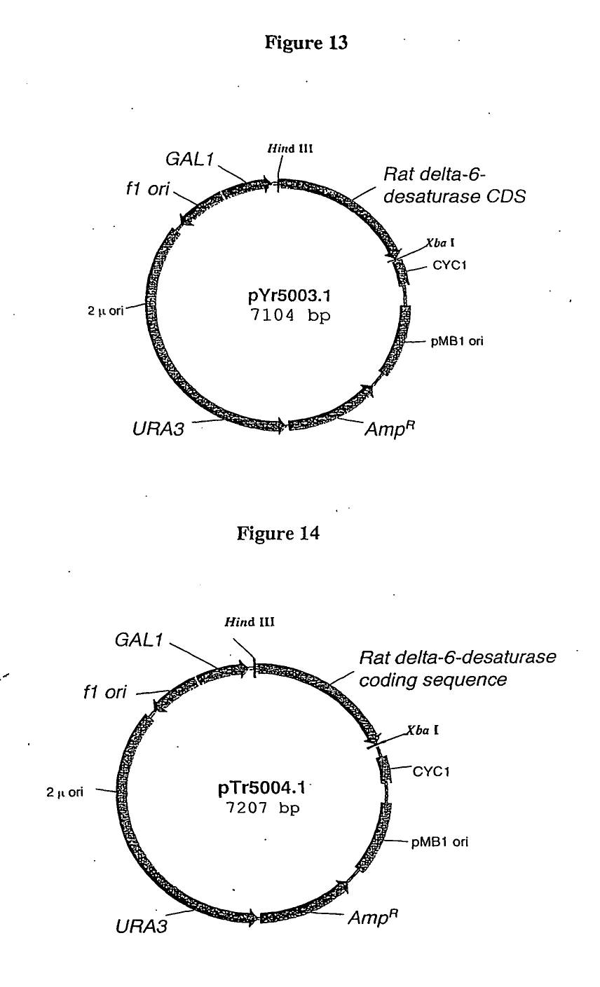

The forward and reverse primers for the rat delta 6 desaturase gene (rD6D-1) were 5′-CACGCGAAGCTTATGGGGAAGGGAGGTAACCAG-3′ and 5′-CACGCGTCTA GATCATTTGTGGAGGTAGGCATC-3′ respectively, for the cloning in the pYES2 vector (Invitrogen, CA). The PCR product contained an HindIII and a XbaI site (underlined) adjacent to the translation initiation and stop codons respectively (indicated by boldface type). The forward primer for cloning rD6D-1 in the pYES2/CT vector which contains a C-terminal tag for protein detection and purification (Invitrogen, CA) was the same as was used for the cloning in the pYES2 vector. The reverse primer was 5′-CACGCGTCTAGATTTGTGGAGGTAGGCATCCAG-3′. This primer does not have a stop codon because a stop codon is present in the pYES2/CT vector after the C-terminal tag. The rD6D-1 gene constructs in pYES2 and pYES2/CT vectors were named pYr5003.1 and pTr5004.1 respectively (FIGS. 13 and 14, respectively).

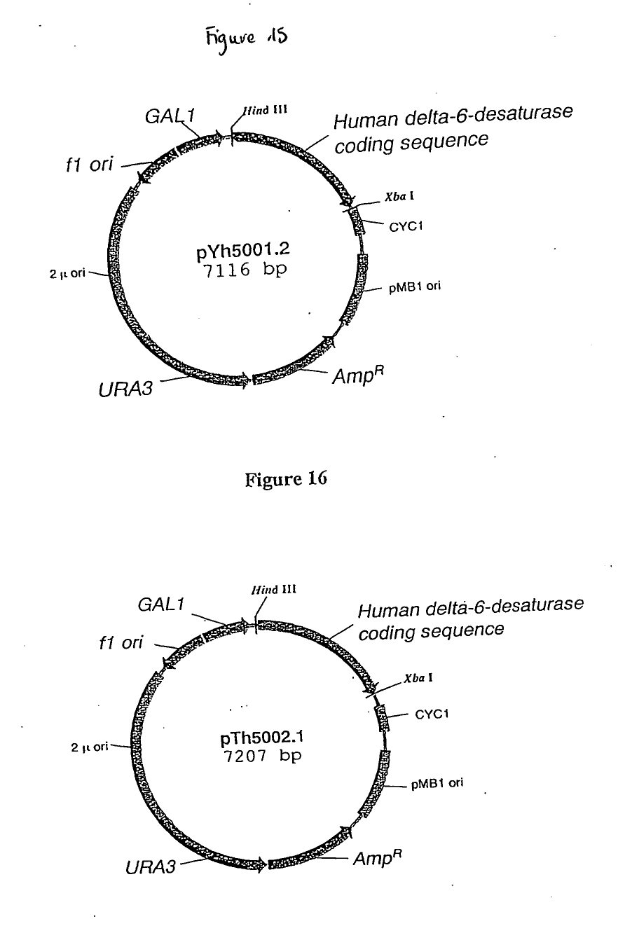

The forward and reverse primers for the human hD6D-1 gene to be cloned in the pYES2 vector were 5′-CACGCGAAGCTTATGGGGAAGGGAGGGAAC-3′ and 5′-CACGACTCTAGAGGGGCTGTGGCTTCATTTGT-3′ respectively. The translation initiation and termination codons are indicated by boldface type. The PCR product contained a HindIII and an XbaI site (underlined). The reverse primer for hD6D-1 to be cloned in the pYES2/CT vector was 5′-CACGCGTCTAGATTTGTGAAGGTAGGCGTCCAG-3′; the forward was the same that was used for the pYES2 construction. The pYES2 and the pYES2/CT constructs containing the hD6D-1 gene were named pYh5001.2 and pTh5002.1 respectively (FIGS. 15 and 16, respectively).

The PCR amplification was conducted in a Perkin-Elmer GeneAMP PCR system 9700 instrument in a 50 μl reaction volume containing: 10 μl from the RT reaction, 0.2 μM of each primer, 1× HF dNTP mix (Clontech, CA), 1× HF PCR reaction buffer (Clontech) and 1× Advantage-HF polymerase mix (Clontech). Samples were first denatured at 94° C. for 1 min followed by amplification using 30 cycles of 30 sec at 94° C., 45 sec at 50° C. and 1.5 min at 72° C. The PCR products were gel-purified using QIAquick gel extraction kit (Qiagen, Germany).

The purified PCR products and the yeast expression vectors pYES2 and pYES2/CT were digested with specific restriction enzymes according to the restriction sites generated during amplification and purified using PCR purification kit (Qiagen). The digested vector and PCR products were ligated and transformed into competent E. coli stain INVαF′ (Invitrogen) and selected on plates containing ampicillin. Selected colonies were amplified and plasmid DNA was isolated using QIAprep spin miniprep kit (Qiagen). All plasmid constructions were confirmed by DNA sequencing analysis. Transfer of the constructions into Saccharomyces cerevisiae strain INVSc1 (Invitrogen) was done by the lithium acetate method Invitrogen) and recombinant yeast cells were selected on uracil-deficient medium.

### Example 6

### Mammalian Desaturase Control Regions

The control region for the rD6D-1 gene was located upstream of the coding portion of the same gene using cloning techniques to walk along the nucleic acid sequence using a rat genomic library (e.g. RDL-4 PvuII). PCR, nested primers and GenomeWalker libraries were used to walk genomic DNA (Clontech, Palo Alto, Calif.).

In particular, genomic DNA was first amplified in the presence of a primer to an adapter sequence and another primer specific to the 5′ end of the coding sequence. The amplified sequences were then subjected to a second round of PCR with the same, or another, adapter primer and a different specific primer internal (i.e. upstream) to the first one.

Table 9 provides examples of forward and reverse primers that were useful in cloning the rD6D-1 control region by nested PCR amplification. Forward adapter primers, AP1 and AP2, used in the 1^{st} and 2^{nd} PCR reactions were supplied in the GenomeWalker kit (Clontech, Palo Alto, Calif.). The reverse downstream and upstream specific primers utilized in the 1^{st} and 2^{nd} PCR reactions, respectively, were designed to correspond and hybridize to the 5′ end of the coding sequence. The translation initiation codon is indicated in boldface type.

TABLE 8

| | |
|---|---|
| 1^{st} PCR Reaction | |
| Forward AP1 | 5′-GTAATACGACTCACTATAGGGC-3′ |
| Reverse | 5′-CAGGTTGTGCTTCTGAATCTCCTC-3′ |
| 2^{nd} PCR Reaction | |
| Forward AP2 | 5′-ACTATAGGGCACGCGTGGT-3′ |
| Reverse | 5′-TCTCCCTGGTTACCTCCCTTCCCCATG-3′ |

The conditions for the first PCR reactions were:

7 cycles at 94° C. for 25 seconds, 72° C. for 4 minutes

32 cycles at 94° C. for 25 seconds, 67° C. for 4 minutes

67° C. for 4 minutes

The conditions for the second PCR reactions were:

7 cycles at 94° C. for 25 seconds, 72° C. for 4 minutes

32 cycles at 94° C. for 25 seconds, 71° C. for 4 minutes

67° C. for 4 minutes

PCR reaction products were recovered, inserted into a TA cloning vector, preferably pCRII (Invitrogen), and then sequenced. Linearized cloning vectors for TA cloning contain a single 3′ deoxythymidine (T) residue overhang to allow for efficient ligation to PCR products with 3′ deoxyadenosine (A) overhangs. DNA products of PCR amplification contain a single 3′ A overhang due to the nontemplate-dependent activity of Taq polymerases.

Subcloning of the rD6D-1 control region following its insertion into the pCRII cloning vector was achieved during PCR amplification using a new set of forward and reverse primers. The oligonucleotide primers used in subcloning the rD6D-1 control region may advantageously comprise additional nucleotide sequences which contain one or more endonuclease recognition sites to facilitate insertion and ligation into an expression vector following PCR amplification. In the present invention, the forward and reverse primers contain a SacI and AhoI restriction site, respectively.

Optionally, an oligonucleotide primer may also contain a translation initiation codon (i.e. positioned downstream at the 5′-end of the reverse primer) which is operatively associated with a heterologous nucleic acid sequence encoding a gene product. In a preferred embodiment of the present invention, the translation initiation codon is not provided within the reverse primer sequence but is supplied instead from the 5′-end of the heterologous nucleic acid sequence which is ligated to the 3′-end of the control region.

Examples of forward and reverse primers that are useful in cloning the rD6D-1 control region from position-1 from the translation initiation codon, ATG, from the TA cloning vector for subsequent insertion into a reporter vector are listed below in Table 9. The endonuclease recognition sites are underlined.

TABLE 9

| | |
|---|---|
| Forward | 5′-CACGACGAGCTCCTGCTGTTCATTCCTTCTGAGA-3′ |
| Reverse | 5′-CACGACCTCGAGGCTGCCTGTCTACCCCGGATGA-3′ |

In a preferred embodiment of the present invention, the control region which is isolated and cloned from the rD6D-1 gene contains a nucleotide sequence (1.6 kb) from nucleotide −1595 to the translation initiation site of the rat desaturase gene, or a portion of that sequence represented by NO: 1.

The hD6D-1 control region was cloned from human genomic DNA. In particular, genomic DNA from Chang cells was amplified in the presence of a reverse primer from position −101 from the translation initiation codon, ATG, and a forward primer approximately 1.4 kb further upstream. Another reverse primer was also used subsequently to clone the hD6D-1 control region from position −1 from the translation initiation codon, ATG, to position −1497 using the same forward primer.

Table 10 provides examples of forward and reverse primers that were useful in cloning the hD6D-1 control region by PCR amplification.

TABLE 10

| | |
|---|---|
| Forward | 5′-TCTCAGGCTCTCCATTTTCA-3′ |
| Reverse | 5′-CTCTTCGCTTTCGGCTTTTG-3′ |
| Reverse | 5′-CACGCGCTGCAGGCTGCCTGCCGACTGTGA-3′ |
| Forward | 5′-GCGGGTACCTGTCAGGGTCTCCATTTTCAAGTG-3′ |
| Reverse | 5′-GCGGGTACCGCTGCCTGCCGACTGTGA-3′ |

PCR reaction products (1.4 and 1.5 kb) were recovered, inserted into a TA cloning vector, preferably pCRII (Invitrogen), and then sequenced. To facilitate insertion and ligation of the control region of 1.4 kb into a reporter vector, the pCRII cloning vector was cut with endonuclease enzymes at the restriction sites KpnI and XhoI, which were already present in the pCRII vector and which flanked the cloning site where the control sequence was inserted. The pCRII construction containing the 1.5 kb fragment of the hD6D control region, was used as DNA template to performed PCR amplification with primers containing restriction site for the endonuclease enzyme KpnI (underlined in Table 10). The reaction product of 1.5 kb, containing the hD6D DNA fragment from position −1 from the ATG to −1497, was inserted into the reporter vector after restriction of both the PRC product and the reporter vector with the endonuclease enzyme KpnI and subsequent ligation. The proper orientation of the inserted fragment was determined by sequencing. In a preferred embodiment of the present invention, the translation initiation codon is not provided within the reverse primer sequence but is supplied instead within the 5′-end of the heterologous nucleic acid sequence which was ligated to the 3′-end of the control region.

In a preferred embodiment of the present invention, the control region isolated and cloned from the hD6D-1 gene contains a nucleotide sequence (1.4 or 1.5 kb) from nucleotide −1497 to −101 or to −1 of the human desaturase gene, or a portion of that sequence represented by SEQ ID NO: 2.

### Example 7

### Cell Transformation and Cat/Luciferase Enzyme Assays

The rat control region (1.6 kb upstream from the ATG) was subcloned in frame in the CAT (Chloramphenicol Acetyl Transferase) reporter vector pCAT-3-Enhancer (Promega Corp., WI) by PCF The forward and reverse primers used were 5′-CACGACGAGCTCCTGCTGTTCATTCCTTCTGAGA-3′ and 5′-CACGACCTCGAGGCTGCCTGTCTACCCGGATGA-3′ respectively. The PCR product contained a SacI site and a XhoI site (underlined) and does not contain the AP2 adapter (use in the cloning process) nor the ATG. The ATG from the CAT gene is used instead.

The PCR amplification was conducted in a Perkin-Elmer GeneAMP PCR system 9700 instrument in a 50 μl reaction volume containing: 5 μg of the plasmid DNA construction pCRII containing the rat desaturase control region, 0.2 μM of each primer, 1× HF dNTP mix (Clontech, CA), 1× HF PCR reaction buffer (Clontech) and 1× Advantage-HF polymerase mix (Clontech). Samples were first denatured at 94° C. for 30 sec followed by amplification using 30 cycles of 30 sec at 94° C. 45 sec at 60° C. and 1.5 min at 72° C. The PCR products were gel-purified using QIAquick gel extraction kit (Qiagen, Germany).

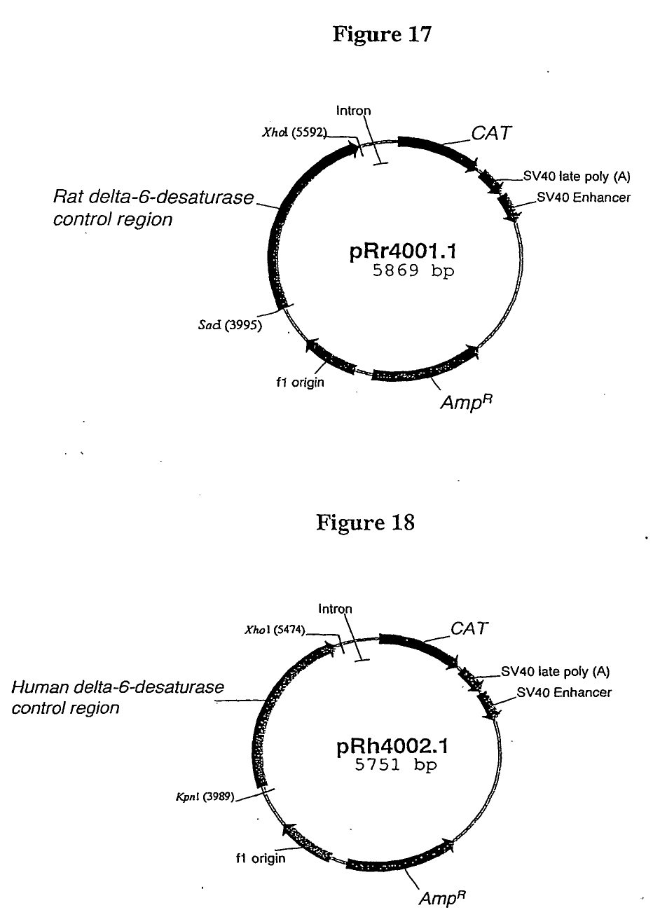

The gel-purified PCR product and the pCAT-3-Enhancer vector were digested with the SacI and XhoI restriction enzymes, ligated and transformed into competent E. coli stain JM109 (Promega). Colonies were selected on plates containing ampicillin. Selected colonies were amplified and plasmid DNA was isolated using QIAprep spin miniprep kit (Qiagen). The transformants were screened by restriction analysis and confirmed by DNA sequencing. The 1.6 kb rat desaturase control region cloned in the pCAT-3-Enhancer vector was named pRr4001.1 (FIG. 17).

The human hD6D-1 control region (1.4 kb) was cloned by PCR in the TA cloning vector pCRII (Invitrogen). The forward and reverse primers used were 5′-TCTCAGGCTCTCCATTTTCA-3′ and 5′-CTCTTCGCTTICGGCTTTTG-3′ respectively. The PCR conditions were similar to those used for the rat control region. The pCRII construction containing the hD6D-1 control region and the pCAT-3-Enhancer vector were digested with the KpnI and the XhoI restriction enzymes. The promoter fragment was gel-purified and ligated in the pCAT-3-Enhancer vector. After transformation into the competent E. coli stain JM109 (Promega), colonies were selected on plates containing ampicillin. Plasmid DNA was isolated using QIAprep spin miniprep kit (Qiagen) from the isolated colonies and screened by restriction analysis and confirmed by DNA sequencing. This construction was named pRh4002.1 (FIG. 18).

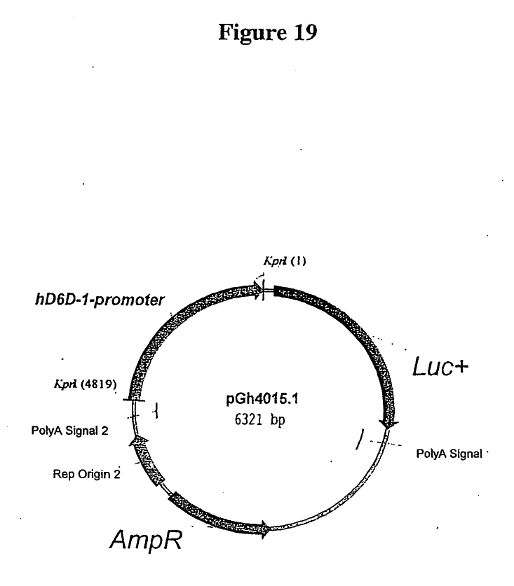

A longer fragment from the hD6D-1 control region (1.5 kb) was also cloned by PCR in the TA cloning vector pCRII (Invitrogen). The forward and reverse primers used were 5′-TCTCAGGCTCTCCATTTTCA-3′ and 5′-CACGCGCTGCAGOCTGCCTGCCGACTGTGA-3′ respectively. The resulting construction was subsequently used as DNA template for PCR amplication with the following forward and reverse primers: 5′-GCGGGTACCTCTC AGGCTCTCCATTTTCAAGTG-3′ and 5′-GCGGGTACCGCTGCCTG CCGACTGTGA-3′. The amplication primers contained restriction site for the endonuclease enzyme KpnI (underlined). The PCR product was digested with the enzyme KpnI before its ligation in the reporter vector pGL-3 Basic, which was also digested with the same enzyme. After transformation into the competent E. coli stain TOP 10 (Invitrogen), colonies were selected on plates containing ampicillin. Plasmid DNA was isolated using QIAprep spin miniprep kit (Qiagen) from the isolated colonies and screened by restriction analysis and confirmed by DNA sequencing. This construction was named pGh4015.1 (FIG. 19).

Cell Transfection:

The cell line ZR-75-1 (ATCC # CRL 1500) or HepG2 (ATCC # HB-8065) was transfected with 5 μg of the plasmid DNA constructions pRr4001.1 or pRh4002.1 using 5 μl of lipofectamine 2000 Reagent (Gibco BRL, Life Technologies, MD) in a 6-well plate as described by the manufacturer. The plasmids pCAT-3-Enhancer (5 μg) or pGL3-Basic (5 μg) and were also transfected as positive and negative controls respectively. In all cases, 5 μg of the plasmid construction pSV-β-Gal (pSV-β-Galactosidase control vector; Promega Corp.) was also co-transfected and used as an internal control to standardize the transfection efficiency between each transfection.

Stable Cell Transfection:

Stable transfection was performed using the HepG2 cell line, which was adapted to grow in tissue culture dishes treated with 0.1% gelatin. The day before transfection, the cells were plated into 60 mm tissue culture dishes, so that they were 85-95% confluent on the day of transfection. For each dish of cells to be transfected, 10 μg of plasmid DNA (pGh4015.1) along with 1 μg of the vector pRSV-NEO (ATCC# 37198), was diluted in 500 μl of Opti-MEM (GIBCO-BRL) without serum. For each dish of cells to be transfected, 10 μl of Lipofectamine 2000 reagent (GIBCO BRL) was diluted in 500 μl of Opti-MEM, and incubated at room temperature for 5 min. The diluted DNA was mixed with the diluted lipofectamine and incubated at room temperature for 20 min. The DNA-lipofectamine complexes (1 ml) were put directly into each dish of cells to be transfected and mixed gently by rocking the dish back and forth The cells were incubated at 37° C. in a CO₂ incubator for 24 h Each transfected dish of cells was then passaged into two 150 mm tissue culture dishes. The following day, geneticin (GIBCO BRL) was added to the culture medium at a concentration of 800 μg/ml. The cells were kept in selection for 4 weeks. Clones were transferred into a 96-well plate (one clone per well) until they reached confluence. Subsequently, the clones were transferred to a 24-well plate before been tested for luciferase activity.

CAT (Chloramphenicol Acetyl Transferase) Enzyme Assay:

For the CAT assays, the transfected cells were harvested 48 h after transfection and cellular protein extracts were prepared using 1× Reporter Lysis Buffer (Promega). The CAT assay was done using the CAT Enzyme Assay System from Promega following the company's protocol. Essentially, about 20 μg of protein extract was incubated with 75 μCi of ¹⁴C-chloramphenicol (NEN, MA) and 25 μg of n-butyryl Coenzyme A provided in the kit. The reaction mixture was incubated at 37° C. for 1 h. The reaction was then stopped by the addition of 300 μl of mixed xylenes. The xylenes phase was extract twice with 100 μl of 0.25 M Tris-HCl (pH 8.0); 200 μl of the upper xylene phase was combined with 10 ml of scintillation fluid (Ready-Safe, Beckman, Calif.) and counted in a liquid scintillation counter. A standard curve was also performed with pure enzyme, at the same time, to ensure that the extracts were diluted enough to give a enzymatic reaction that is in the linear range of the standard curve.

Beta-Galactosidase Enzyme Assay:

The beta-galactosidase enzymatic activity was used as an internal control to standardize the transfection efficiency between transfections. To do the assay, the same amount of protein extract (20 μg) used for the CAT assay was diluted with 1× Reporter Lysis Buffer to 150 μl and incubated with the same volume of 2× Assay Buffer (Beta-Galactosidase Enzyme Assay System, Promega) which contained 200 mM sodium phosphate buffer, pH 7.3,2 mM MgCl₂, 100 mM beta-mercaptoethanol and 1.33 mg/ml ONPG (o-nitrophenyl-beta-D-galactopyranoside). The reaction mixture was incubated at 37° C. for 30 min to 1 h (until a faint yellow color has developed). The reaction was stopped by addition of 500 μl of 1 M sodium carbonate and the absorbance was read at 420 nm.

Luciferase Activity Assay:

The luciferase assays were done using the Luciferase Assay System from Promega (Promega, Wis.). Briefly, the cells grown in a 24well plate, were washed with PBS (Ca²⁺ and Mg²⁺ free) and lysed with about 100 μl of 1× CCLR (Cell Culture Lysis Reagent, Promega). Twenty μl of cell lysate was dispensed into a luminometer tube. The tube was placed in the luminometer (Berthold #9707, Monolight 3010, PharMingen, San Diego, Calif.) and 100 μl of Luciferase Assay Reagent (Promega) was injected into the tube. The luminometer was programmed to perform a 3-second measurement delay followed by a 15-second measurement read for luciferase activity. The results were expressed in Relative Light Unit per 15 seconds (RLU/15s).

Results:

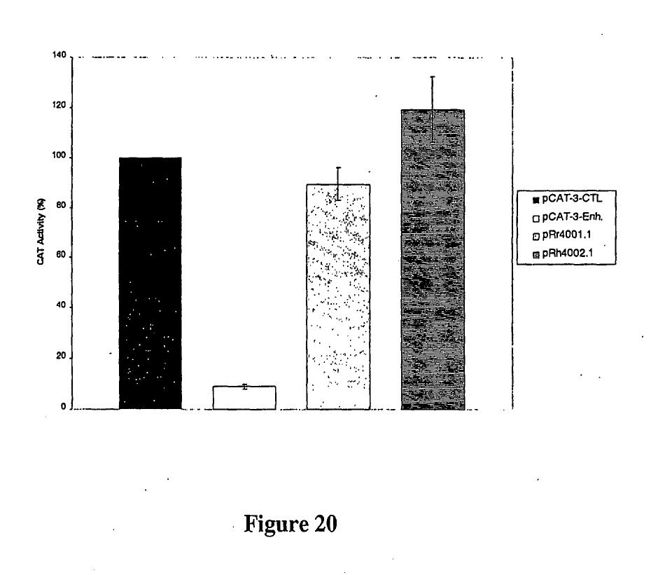

Refer to FIG. 20.

Conclusions:

The results of the transfections done in the human cell line ZR-75-1 shown in FIG. 20 indicate that the human and the rat delta-6-desaturase-1 gene control regions are as active as the SV40 promoter in this cell line. The fact that the rat control region is slightly less active than the human could be explained by the lack of species specific factors.

### Example 8

### Regulation of the HD6D-1 Promotor Activity By Pufa

Previous Northern blot results indicated possible regulation of the hD6D-1 promoter activity by PUFAs. We used 2 stable clones transfected with 1.5 kb fragment of the hD6D-1 control region fused to the luciferase coding region, to address the possible regulation of the promoter activity by PUFAs.

Chemicals:

The DHA was obtained from Sigma-Aldrich. The stock solution was diluted in ethanol to a final concentration of 140 mM.

Cell Culture and Incubation:

The HepG2 stable clone 19 and 72, were plated in a 24-well plate (one plate per clone) treated with 0.1% gelatin, containing α-MEM supplemented with 10% FBS (Fetal Bovine Serum) and 200 μg/ml of geneticin (all from GIBCO BRL). When the cells reached about 80% confluence, the culture medium was replace with Williams' medium E (GIBCO) containing insulin (1 μM), dexamethasone (1 nM) and 0.27% BSA (Bovine Serum Albumin) all from Sigma. Six wells for each plate were treated with DHA at a concentration of 0, 25, 50 and 100 μM. After an incubation time of 6, 24 and 48 h, the wells were washed with PBS and the cells lysed with 100 μl of 1× CCLR (Promega). The luciferase assays were performed using a single tube luminometer using the Luciferase Assay System from Promega as indicated above.

Results:

Treatment of the clones with DHA, dramatically decreases the luciferase activity which indicates that DHA directly or indirectly affects the transcription activity of the 1.5 kb fragment from the hD6D-1 control region present in the plasmid construction pGh4015.1 (Table 11).

TABLE 11
Luciferase activity from stable transfected cells treated with DHA.

| | RLU/15 s | | |
|---|---|---|---|
| DHA Concentration | Incubation time | | |
| (μM) | 6 h | 24 h | 48 h |
| Clone 19 | | | |
| 0 | 5.33E+06 | 1.92E+07 | 2.37E+07 |
| 25 | 3.91E+06 | 8.33E+06 | 1.35E+07 |
| 50 | 3.96E+06 | 6.32E+06 | 8.73E+06 |
| 100 | 2.11E+06 | 3.87E+06 | 4.16E+06 |
| Clone 72 | | | |
| 0 | 1.40E+06 | 8.86E+06 | 1.11E+07 |
| 25 | 8.27E+05 | 2.66E+06 | 3.68E+06 |
| 50 | 6.69E+05 | 1.43E+06 | 2.18E+06 |
| 100 | 3.07E+05 | 8.55E+05 | 9.80E+05 |

### Example 9

### Functional Analysis of Yeast Cells Transformed With Rat Delta-6-Desaturase gene (rD6D-1) With (pTr5004.1) or without (pYr5003.1) V5/6×His Tags

Chemicals and Radiochemicals:

Tris buffer, fatty acid free bovine serum albumin, tergitol, carbohydrates, amino acids and fatty acids were obtained from Sigma-Aldrich Canada (Oakville, ON, Canada). Yeast nitrogen base without amino acids was purchased from Difco (Becton Dickinson Co; Sparks, Md. U.S.A.) All organic solvents (HPLC grade) were obtained from Fisher-Scientific (Fair Lawn, N.J., U.S.A.).

[1-¹⁴C]-alpha-linolenic acid and [1-¹⁴C]-dihomogamma-linolenic acid (99% radiochemical purity; specific activity: 51, 52 and 52 μCi/μmol, respectively), were purchased from NEN (Boston, USA). These fatty acids were saponified with KOH (0.1 M) and dissolved in SC-U medium (minimum medium without uracil) with 1% tergitol.

Incubation:

Transformed Saccharomyces cerevisiae cells with a rat fatty acid delta-6-desaturase gene (rD6D-1), with (pTr5004.1) or without (pYr5003.1) V5/6×His tags, were incubated in 125 mL Erlenmeyers containing 10 mL of SC-U medium (1% raffinose), 1% tergitol (O.D. 0.4, approximately 3.2×10⁶ cells) and the potassium salts of either [1-¹⁴C]-linoleic, [1-¹⁴C]-alpha-linolenic or [1-¹⁴C]-dihomogamma-linolenic acids. After 5 h incubation in an orbital incubator at 280 rpm and 30° C., cells reached the log phase and the transgene expression was induced with galactose (2% final concentration). Yeast were further incubated for 19 h until they were harvested by centrifugation at 5000× g for 10 minutes at 4° C.

Cell were washed with Tris buffer (100 mM, pH 8.0) containing 0.1% BSA and total lipids were extracted as described below. The radioactivity from aliquots of the supernatant and the cells was determined by liquid scintillation counting using a LS6500-Scintillation System (Beckman).

The host yeast transformed with pYES2 vector alone was used as negative control.

Delta-6-Desaturase Activity of Transformed Yeast in Various Culture Conditions

Transformed yeast were incubated as described above with different concentrations and specific activities of [1-¹⁴C]-linoleic acid (range from 2 to 500 μM and 0.4 to 20 μCi/μmol, respectively) or with 25 μM [1-¹⁴C]-linoleic acid at 15° C. for 48 h. In another experiment, cells were incubated with 25 μM of [1-¹⁴C]-alpha-linolenic acid and harvested at 2, 5 and 19 h after the transgene induction with galactose.

Lipid Extraction:

Total lipids were extracted from cells with chloroform/methanol (2:1, v/v) according to the method of Folch et al., 1957, J. Biol. Chem., 226: 497-509. The total lipid extracts were methylated using boron trifluoride in methanol at 90° C. for 30 min (Morrison W. R and Smith L. M., 1964, J. Lipid Res., 5: 600-608). The resultant methyl esters (FAMEs) were analyzed as described below.

High Performance Liquid Chromatography (HPLC) Analysis:

Analyses of radiolabelled FAME were carried out on a Hewlett Packard (1090, series II) chromatograph equipped with a diode array detector set at 205 nm, a radioisotope detector (model 171, Beckman, Fullerton, Calif.) with a solid scintillation cartridge (97% efficiency for ¹⁴C-detection) and a reverse-phase ODS (C-18) Beckman column (250 mm×4.6 mm i.d.; 5 μm particle size) attached to a pre-column with a μBondapak C-18 (Beckman) insert. FAME were separated isocratically with acetonitrile/water (95:5, v:v) at a flow rate of 1 mL/min and were identified by comparison with authentic standards.

The eluted FAME were collected and the solvent evaporated. FAME were re-dissolved in hexane for further analysis by gas chromatography.

Gas Chromatography (GC) Analysis:

The FAME profile was determined using a Hewlett Packard Gas Chromatograph equipped with an interfaced ChemStation, a flame-ionization detector and a 30 m×0.25 mm i.d. fused silica column (HP-wax, cross linked polyethylene glycol, film thickness 0.25 μm). He was the gas carrier. The temperature of the injector and detector was set at 225 and 250° C., respectively. The oven temperature was programmed as follows: started at 180° C. and held it for 1 min, increased 4° C./min until 190° C. was reached and held it for 7 min, increased 10° C./min up to 200° C. and held it for 5 min, increased 25° C./min until 215° C. was reached and this final temperature was maintained for 17.9 min. FAME were identified by comparison with authentic standards (de Antueno et al., 1994, Lipids, 29: 327-331).

Results:

There was a modest incorporation of radiolabelled linoleic acid, (18:2n-6, LA) and alpha-linolenic acid (18:3n-3, ALA), both substrates of delta-6-desaturase, into the transformed yeast after 24 h incubation with 500 μM of each fatty acid (Table 12).

TABLE 12
Percent of radioactivity recovered in Saccharomyces cerevisiae cells
transformed with pYr5003.1 and pYES2, after the incubation for 24 h
with 500 μM linoleic acid ([1-¹⁴C]-18:2n-6) and alpha-linolenic acid
([1-¹⁴C]-18:3n-3).

%

| PLASMID | FATTY ACID (2 uCi) | Cells | Supernatant |
|---|---|---|---|
| pYr5003.1 | [1-¹⁴C]-18:2n-6 | 2.23 | 46.75 |
| pYES2 | [1-¹⁴C]-18:2n-6 | 2.34 | 48.26 |
| pYr5003.1 | [1-¹⁴C]-18:3n-3 | 3.19 | 28.35 |
| pYES2 | [1-¹⁴C]-18:3n-3 | 5.53 | 29.63 |

Values are the mean (dispersion. 10%) of two yeast cultures derived form the same transformed colony.
O.D₆₀₀: 19.35 ± 1.96

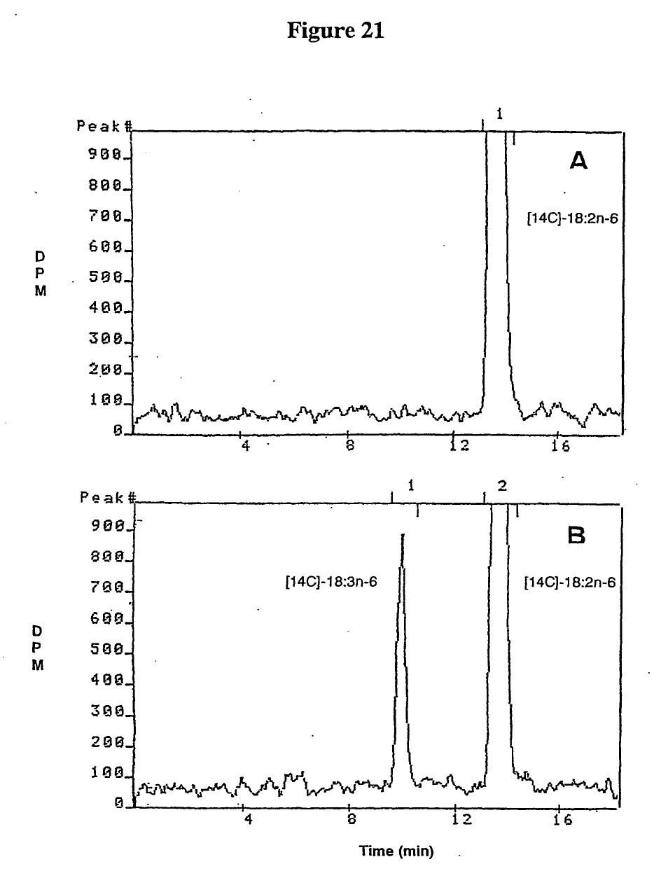

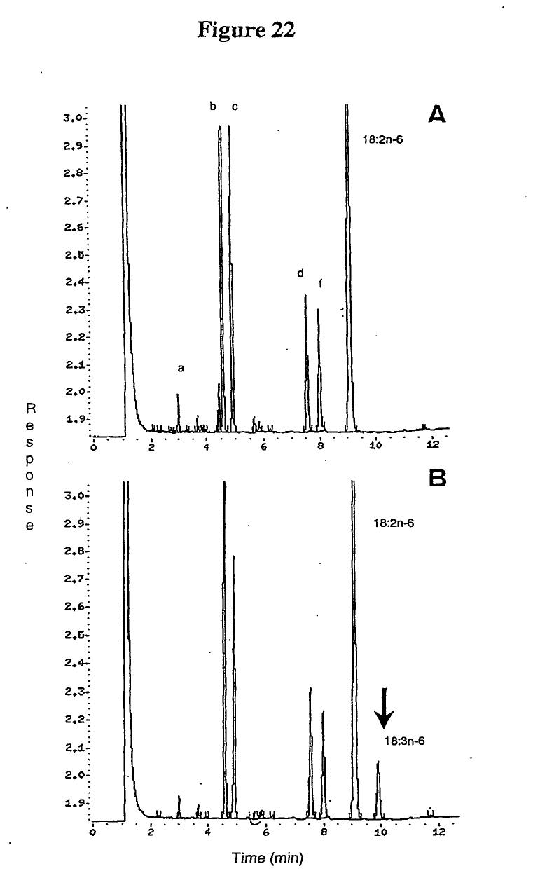

The analyses by two different methods, reverse phase-high performance liquid chromatography (RP-HPLC) and capillary column-gas chromatography (GC), revealed that linoleic acid was converted into gamma-linolenic acid (18:3n-6, GLA) in yeast transformed with pYr5003.1 (Table 13; FIGS. 21 and 22 panel B). Such enzyme activity was not detected in the host yeast transformed with pYES2 alone (FIGS. 21 and 22, panel A).

TABLE 13
Percent of substrate conversion and enzyme activity
in Saccharomyces cerevisiae cells transformed with pYr5003.1,
19 h after the induction with galactose.

| PLASMID | FATTY ACID Substrate (500 μM) | % | pmol/h/10⁶cells |
|---|---|---|---|
| pYr5003.1 | [1-¹⁴C]-18:2n-6 | 14.21 | 5.71 |
| pYr5003.1 | [1-¹⁴C]-18:3n-3 | 21.61 | 10.94 |

Values are the mean (dispersion. 10%) of two yeast cultures derived form the same transformed colony.
O.D₆₀₀: 19.35 ± 196

The percent uptake of radiolabelled linoleic acid (18:2n-6) by transformed yeast slightly varied with the fatty acid specific activity (ratio between radioactivity, in μCi, and concentration in μM; Table 14) with no significant changes in the conversion of linoleic acid to gamma-linolenic acid (Table 15).

TABLE 14
Percent of radioactivity recovered in Saccharomyces cerevisiae cells
transformed with pYr5003.1 after the incubation for 24 h with linoleic
acid ([1¹⁴C]-18:2n-6) at different concentrations and specific activities.

| Concentration | Radioactivity | Specific Activity | % |
|---|---|---|---|
| (μM) | (μCi) | (μCi/μmol) | Average |
| 500 | 2 | 0.4 | 1.48 |
| 50 | 2 | 4.0 | 1.76 |
| 25 | 2 | 8.0 | 2.29 |
| 10 | 2 | 20.0 | 2.08 |
| 500 | 1 | 0.2 | 1.79 |
| 50 | 1 | 2.0 | 2.16 |
| 25 | 1 | 4.0 | 2.18 |
| 10 | 1 | 10.0 | 1.95 |

Values are the mean (dispersion. 10%) of two yeast cultures derived form the same transformed colony
O.D₆₀₀: 21.92 ± 1.25

TABLE 15
Percent of linoleic acid (18:2n-6) conversion to gamma-linolenic acid
(18:3n-6) in Saccharomyces cerevisiae cells transf rmed with
pYr5003.1, 24 h after the incubation with different concentrations of
[1-¹⁴C]-18:2n-6 and 19 h after the induction with galactose.

| Concentration | Radioactivity | Specific Activity | % |
|---|---|---|---|
| (μM) | (μCi) | (μCi/μmol) | Average |
| 500 | 2 | 0.4 | 17.27 |
| 50 | 2 | 4.0 | 16.04 |
| 25 | 2 | 8.0 | 14.35 |
| 10 | 2 | 20.0 | 15.52 |
| 500 | 1 | 0.2 | 15.38 |
| 50 | 1 | 2.0 | 14.84 |
| 25 | 1 | 4.0 | 14.18 |
| 10 | 1 | 10.0 | 13.92 |

Values are the mean (dispersion. 10%) of two yeast cultures derived form the same transformed colony.
O.D₆₀₀: 19.35 ± 19.6

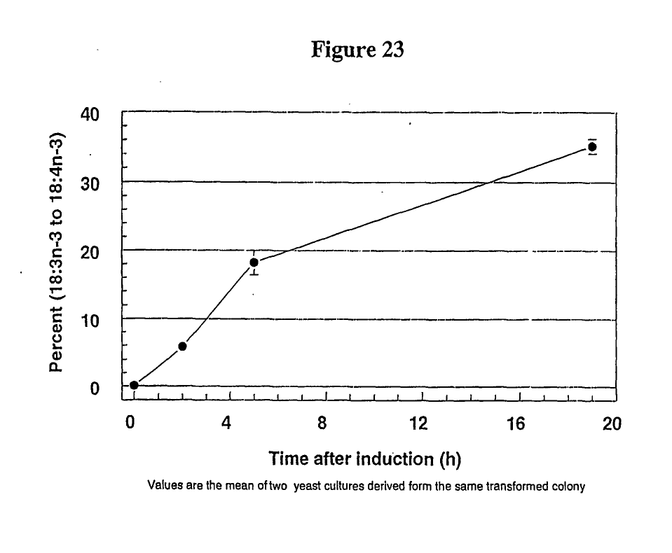

The uptake of radiolabelled alpha-linolenic acid (18:3n-3) and its conversion to 18:4n-6 were 1.5-fold higher than those detected for linoleic acid (Tables 12 and 13). The delta-6 desaturation of alpha-linolenic acid linearly increased with the induction time (FIG. 23). These findings showed that, in transformed yeast under these experimental conditions, delta-6-desaturase activity on alpha-linolenic can be accurately detected within the first 2 h of the induction of the gene expression with galactose.

No desaturation activity was detected when transformed yeast with pYr5003.1 were incubated with [1-¹⁴C]-dihomogamma-linolenic acid (20:3n-6).

There was a reduction of approximately 50% in the conversion of linoleic acid into gamma-linolenic acid in transformed yeast with delta-6-desaturase-V5/6xHis tags, pTr5004.1, when compared to that without tags, pYr5003.1 (Table 16).

TABLE 16
Percent of lin leic acid c nverted into gamma-linolenic acid in
transformed yeast with delta 6-desaturase rat gene (rD6D-1)
with (pTr5004.1) and without (pYr5003.1) V5/6xHis tags.
Transformed Yeast

| pTr5004.1 (+ tags) | pYr5003.1 (− tags) |
|---|---|
| 6.35 | 12.06 |

Values are the mean (dispersion. 10%) of two yeast cultures derived form the same transformed colony.
O.D₆₀₀ 9.0 ± 0.94

Approximately 11% desaturation of linoleic acid was observed in transformed yeast with pYr5003.1 incubated at 15° C. for 48 h.

Conclusion:

The functional analysis of the transgene construct pYr5003.1 in Saccharomyces cerevisiae revealed that the gene encodes a fatty acid delta-6-desaturase which is active on linoleic acid (18:2n-6) and alpha-linolenic acid (18:3n-3) under different experimental conditions.

### Example 10

### Functional Analysis of Yeast Cells Transformed With Human Delta-6 Desaturase Gene (hD6D-1) with (pTh5002.1) or without (pYh5001.2) V5/6×His tags

Chemicals and Radiochemicals:

Tris buffer, fatty acid free bovine serum albumin, tergitol, carbohydrates, amino acids and fatty acids were obtained from Sigma-Aldrich Canada (Oakville, ON, Canada). Yeast nitrogen base without amino acids was purchased from Difco (Becton Dickinson Co; Sparks, Md., U.S.A.). All organic solvents (HPLC grade) were obtained from Fisher-Scientific (Fair Lawn, N.J., U.S.A.).

[1-¹⁴C]-Linoleic acid, [1-¹⁴C]-alpha-linolenic acid and [1-¹⁴C]-dihomogamma-linolenic acid (99% radiochemical purity; specific activity: 51, 52 and 52 μCi/μmol, respectively), were purchased from NEN (Boston, Mass., U.S.A.). These fatty acids were saponified with KOH (0.1 M) and dissolved in SC-U medium (minimum medium without uracil) with 1% tergitol.

Incubation:

Transformed Saccharomyces cerevisiae with a human fatty acid delta-6-desaturase gene (hD6D-1), with (pTh5002.1) or without V5/6xHis tags (pYh5001.2), were incubated in a 125 ml Erlenmeyer containing 10 ml of SC-U medium (1% raffinose), 1% tergitol (O.D.₆₀₀0.4, approximately 3.2×10⁶ cells) and 25 μM (1 μCi) potassium salts of either [1-¹⁴C]-linoleic or [1-¹⁴C]-alpha-linolenic. Cells without V5/6×His tags (pYh5001.2) were also incubated with [1-¹⁴C]-dihomogamma-linolenic and [1-¹⁴C]-oleic acids. Yeast transformed with a rat delta-6-desaturase gene, with (pTr5004.1) or without V5/6×His tags (pYr5003. 1) were used as controls. After 5 h incubation in an orbital incubator set at 280 rpm and 30° C., cells reached the log phase and the transgene expression was induced with galactose (2% final concentration). Yeast were further incubated for 19 h (O.D.₆₀₀ approximately 9.55±1.62) until they were harvested by centrifugation at 5000× g for 10 minutes at 4° C.

Cell were washed with Tris buffer (100 mM, pH 8.0) containing 0.1% BSA and total lipids were extracted as described below. The radioactivity from aliquots of the supernatant and the cells at time zero and after the incubation was determined by liquid scintillation counting using a LS6500-Scintillation System (Beckmnan).

The host yeast transformed with the pYES2 vector alone was used as negative control (data not shown).

Lipid Extraction:

Total lipids were extracted from cells with chloroform/methanol (2:1, v/v) according to the method of Folch et al., 1957, J. Biol. Chem., 226: 497-509. The total lipid extracts were methylated using boron trifluoride in methanol at 90° C. for 30 min (Morrison W. R. and Smith L. M., 1964, J. Lipid Res., 5: 600-608). The resultant methyl esters (FAME) were analyzed and are described below.

High Performance Liquid Chromatography (HPLC) Analysis:

Analyses of radiolabelled FAME were carried out on a Hewlett Packard (1090, series II) chromatograph equipped with a diode array detector set at 205 nm, a radioisotope detector (model 171, Beckman, Fullerton, CA) with a solid scintillation cartridge (97% efficiency for ¹⁴C-detection) and a reverse-phase ODS (C-18) Beckman column (250mm×4.6 mm i.d.; 5 μm particle size) attached to a pre-column with a μBondapak C-18 (Beckman) insert. FAME were separated isocratically with acetonitrile/water (95:5, v:v) at a flow rate of 1 ml/min and were identified by comparison with authentic standards.

Results:

Fatty Acid Uptake:

The total radioactivity recovered from the administered [1-¹⁴C]-linoleic acid or [1-¹⁴]-alpha-linolenic acid in the transformed yeast and in the supernatant after 24 h incubation was low (Table 17). This indicated that the cells were able to uptake octadecadienoic and octadecatrienoic acids and metabolize them, probably through β-oxidation mechanisms (Van Roermund et al., 1998, EMBO J., 17: 677-687). Conversely, 65-70% of the total radioactivity of the administered [1-¹⁴C]-dihomogamma-linolenic acid or [1-¹⁴C]-oleic acid remained in the supernatant and only 1.0-1.4% of the administered radioactivity was recovered in yeast (data not shown).

TABLE 17
Percent of radioactivity recovered in Saccharomyces cerevisiae
cells transformed with pYh5001.2, pYr5003.1, pTh5002.1 or
pTr5004.1 after the incubation f r 24 h with 25 μM linoleic acid
([1-¹⁴C]-18:2n-6) or alpha-linolenic acid ([1-¹⁴C]-18:3n-3).

| | | % Average | |
|---|---|---|---|
| PLASMID | FATTY ACID (1 μCi) | Cells | Supernatant |
| pYh5001.2 (human; − tags) | [1-¹⁴C]-18:2n-6 | 1.66 | 28.91 |
| pYr5003.1 (rat; − tags) | [1-¹⁴C]-18:2n-6 | 2.26 | 25.46 |
| pTh5002.1 (human; + tags) | [1-¹⁴C]-18:2n-6 | 2.92 | 24.13 |
| pTr5004.1 (rat; + tags) | [1-¹⁴C]-18:2n-6 | 3.75 | 17.60 |
| pYh5001.2 (human; − tags) | [1-¹⁴C]-18:3n-3 | 2.05 | 12.36 |
| pYr5003.1 (rat; − tags) | [1-¹⁴C]-18:3n-3 | 2.83 | 11.82 |
| pTh5002.1 (human; + tags) | [1-¹⁴C]-18:3n-3 | 3.86 | 11.34 |
| pTr5004.1 (rat; + tags) | [1-¹⁴C]-18:3n-3 | 3.75 | 9.82 |

Values are the mean (dispersion 10%) of two yeast cultures derived form the same transformed colony.
The radioactivity recovered in cells and supernatant is based on the total radioactivity administered at time zero.
O.D.₆₀₀: 13.47 ± 1.88 (average S.D)

There were no major differences in the total radioactivity recovered from both, [1-¹⁴C]-linoleic and [1-¹⁴C]-alpha-linolenic acids, in any of the yeast strains transformed with either human or rat fatty acid delta-6-desaturase genes.

Fatty Acid Desaturation:

Table 18 shows the percent conversion of [1-¹⁴C]-linoleic acid (18:2n-6) to [1-¹⁴C]-gamma-linolenic acid (18:3n-6) and [¹-¹⁴C]-alpha-linolenic acid to 18:4n-3 acid. Yeast transformed with pYh500.2 (human; -tags) showed a lower activity for linoleic acid and alpha-linolenic acid, respectively, than the yeast transformed with pYr5003.1 (rat; -tags) .

TABLE 18
Percent of substrate conversion in Saccharomyces cerevisiae
transformed with pYh5001.2, pYr5003.1, pTh5002.1 or
pTr5004.1, after the incubation for 24 h with 25 μM linoleic
acid ([1-¹⁴C]-18:2n-6), alpha-linolenic acid
([1-¹⁴C]-18:3n-3), oleic acid ([1-¹⁴C]-18:1n-9) or
dihomogamma-linolenic ([1-¹⁴C]-20:3n-6).

| PLASMID | FATTY ACID (1 μCi) | % |
|---|---|---|
| pYh5001.2 (human; − tags) | [1-¹⁴C]-18:2n-6 | 4.00 |
| pYr5003.1 (rat; − tags) | [1-¹⁴C]-18:2n-6 | 13.85 |
| pTh5002.1 (human; + tags) | [1-¹⁴C]-18:2n-6 | 5.14 |
| pTr5004.1 (rat; + tags) | [1-¹⁴C]-18:2n-6 | 5.15 |
| pYh5001.2 (human; − tags) | [1-¹⁴C]-18:3n-3 | 15.21 |
| pYr5003.1 (rat; − tags) | [1-¹⁴C]-18:3n-3 | 37.99 |
| pTh5002.1 (human; + tags) | [1-¹⁴C]-18:3n-3 | 17.53 |
| pTr5004.1 (rat; + tags) | [1-¹⁴C]-18:3n-3 | 19.06 |
| pYh5001.2 (human; − tags) | [1-¹⁴C]-18:1n-9 | ND |
| pYr5003.1 (rat; − tags) | [1-¹⁴C]-18:1n-9 | ND |
| pYh5001.2 (human; − tags) | [1-¹⁴C]-20:3n-6 | ND |
| pYr5003.1 (rat; − tags) | [1-¹⁴C]-20:3n-6 | ND |

Values are the mean (dispersion. 10%) of two yeast cultures derived form the same transformed colony.
ND: not detected

In yeast with the V5/6×His-tagged rat transgene, the desaturation of both substrates was reduced by 50% when compared to yeast without the tagged gene, whereas no major differences were observed between the yeast transformed with the human desaturase gene with or without the tags. The delta-6 desaturation of [1-¹⁴C]-oleic acid (18:1n-9) to 18:2n-9 and the delta-5-desaturation on [1-¹⁴C]-dihomogamma-linolenic acid were not detected in both yeast strains transformed with either the non-tagged human or rat genes.

Conclusion:

Functional analysis experiments on Saccharomyces cerevisiae transformed with pYh5001.2 (without V5/6×His tags) or pTb5002.1 (with V5/6×His tags) revealed that the transgenes encode a human fatty acid delta-6desaturase, as functionally distinct from a delta-5-desaturase, which is active on linoleic acid (18:2n-6) and alpha-linolenic acid (18:3n-3).

### Example 11

### Functional Analysis of Saccharomyces cerevisiae Spheroplast Transformed with Rat Delta 6 Desaturase Gene (rD6D-1)

Chemicals and Radiochemicals:

Tris buffer, fatty acid free bovine serum albumin, tergitol, carbohydrates, sorbitol, amino acids, fatty acids, Lyticase and DDT (dithiothreitol) were obtained from Sigma-Aldrich Canada (Oakville, ON, Canada). Yeast nitrogen base without amino acids was purchased from Difco (Becton Dickinson Co; Sparks, Md., U.S.A.). All organic solvents (HPLC grade) were obtained from Fisher-Scientific (Fair Lawn, N.J., U.S.A.).

[1-¹⁴C]-alpha-linolenic acid (18:3n-3; 99% radiochemical purity; specific activity: 52 μCi/μmol), was purchased from NEN (Boston, Mass., U.S.A.). Δ^{9,21,15,18}[1-¹⁴C]-tetracosatetraenoic acid (24:4n-6; 99% radiochemical purity; specific activity: 55 μCi/μmol) was obtained from ARC (St Louis, Mo., U.S.A.). These fatty acids were saponified with KOH (0.1 M) and dissolved in SC-U medium (minimum medium with 1% raffinose and without uracil) with 1% tergitol.

Spheroplast Preparation:

Saccharomyces cerevisiae cells transformed with pYr5003.1 were grown in SC-U medium with 1% raffinose and 1% galactose to induce the expression of the gene that encodes the fatty acyl delta 6 desaturase. After 16 h incubation, cells were centrifuged at 2060×g for 5 min at 4° C., washed once with distilled water and centrifuged again. The volume and weight of the cell pellet were measured. Cells were suspended (1:2, w/v) in 0.1 M Tris.SO₄ (pH 9.4), 10 mM DTT and incubated at 30° C. After 10 min incubation, the cell pellet was obtained by centrifugation, washed once (1:20, w/v) with 1.2 M sorbitol and suspended (1:1, w/v) in 1.2 M sorbitol, 20 mM phosphate buffer (pH: 7.4) as described elsewhere (Daum et al., 1982, J. Biol. Chem., 257: 13028-13033). The 15,800×g (1 min) supernatant of Lyticase was added to the cell suspension at a concentration of 2000 U/mL and the suspension incubated at 30° C. with 50 rpm shaking. Conversion to spheroplasts was checked after 40 min incubation by diluting the suspension with distilled water followed by observation under the microscope (Schatz G. and Kovac L., 1974, Methods in Enzymology, 3 1A: 627-632). After 70 min incubation, approximately 90% of the cells were converted to spheroplasts.

Incubation:

Spheroplasts were harvested by centrifugation at 2060×g for 5 min at 4° C. and washed once with 1.2 M sorbitol. Spheroplasts and whole cells (controls) were suspended in SC-U medium with 1% raffinose, 1% tergitol, 1.2 M sorbitol and 2% galactose to maintain the induction conditions and to give an O.D.₆₀₀, reading of approximately 2.2. Both whole yeast and spheroplast suspensions were divided into two groups. One group was maintained in the same medium. The second group was centrifuged and the cell pellet was resuspended in medium without sorbitol. Ten ml aliquots of all the groups (spheroplasts and whole cells, with or without sorbitol) were incubated with 2 μM (1 μCi) of delta-6desaturase substrates, [1-¹⁴C]-alpha-linolenic or [1-¹⁴C]-24:4n-6 at 30° C. in an orbital incubator at 280 rpm. After 30 and 150 min incubation an O.D.₆₀₀ reading was taken, spheroplasts and whole cells were harvested by centrifugation and washed with Tris buffer (100 mM, pH 8.0) containing 0.1% BSA. Total lipids were extracted as described below. The radioactivity from aliquots of the supernatant, spheroplasts and whole cells was determined by liquid scintillation counting using a LS6500-Scintillation System (Beckmnan).

Lipid Extraction:

Total lipids were extracted from cells with chloroform/methanol (2:1, v/v) according to the method of Folch et al., 1957, J. Biol. Chem., 226: 497-509. The total lipid extracts were methylated using boron trifluoride in methanol at 90° C. for 30 min. The resultant methyl esters (FAME) were analyzed as described below.

High Performance Liquid Chromatography (HPLC) Analysis:

Analyses of radiolabelled FAME were carried out on a Hewlett Packard (1090, series II) chromatograph equipped with a diode array detector set at 205 nm, a radioisotope detector (model 171, Beckman, Fullerton, CA) with a solid scintillation cartridge (97% efficiency for ¹⁴C-detection) and a reverse-phase ODS (C-18) Beckman column (250 mm×4.6 mm i.d.; 5 μm particle size) attached to a pre-column with a μBondapak C-18 (Beckman) insert. FAME were separated isocratically with acetonitrile/water (95:5, v:v) at a flow rate of 1 ml/min and were identified by comparison with authentic standards.

The eluted FAME were collected and the solvent evaporated. FAME were re-dissolved in hexane for further analysis by gas chromatography.

Results:

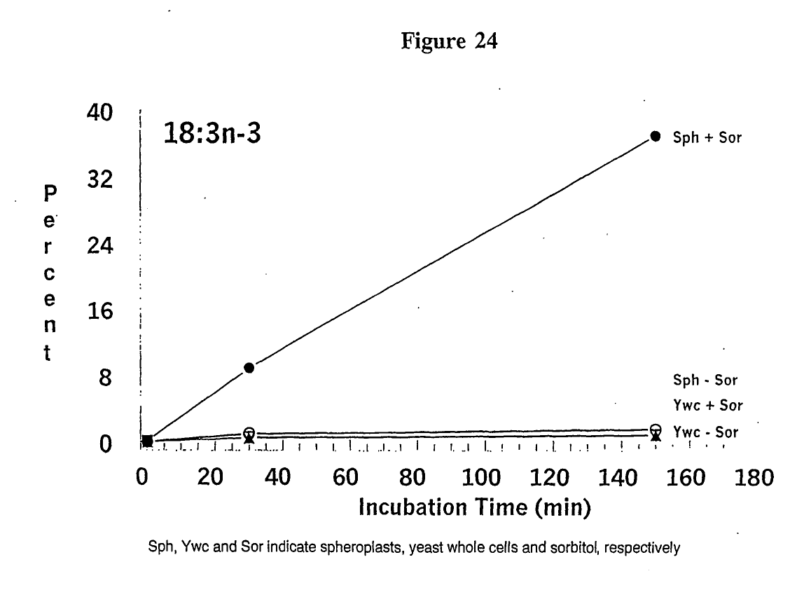

There was a significantly higher uptake of [1-¹⁴C]-alpha-linolenic by spheroplasts at either 30 or 150 min of incubation in the presence of 1.2 M sorbitol when compared to whole cells. Only 1.51% of the total radioactivity provided in the medium without sorbitol was recovered in spheroplasts. In the whole yeast, the low uptake of [1-¹⁴C]-alpha-linolenic acid (0.85%) was not altered by the presence of sorbitol in the medium (FIG. 24).

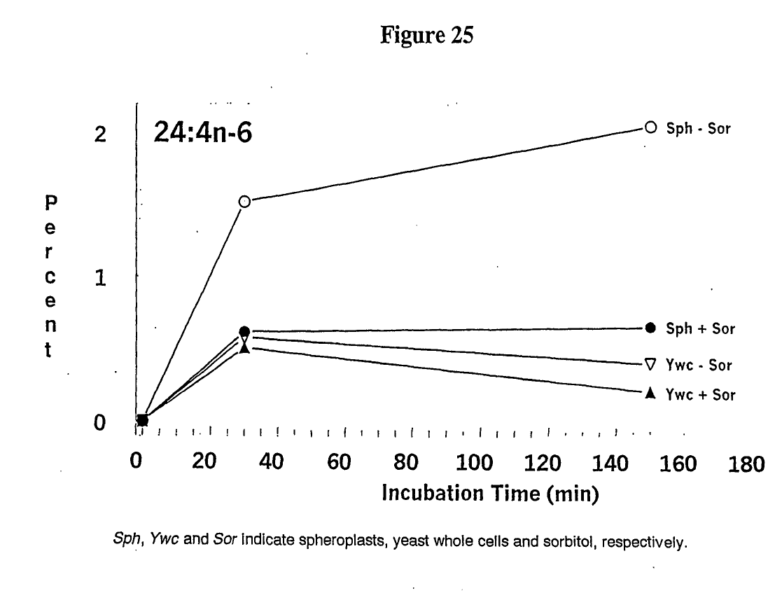

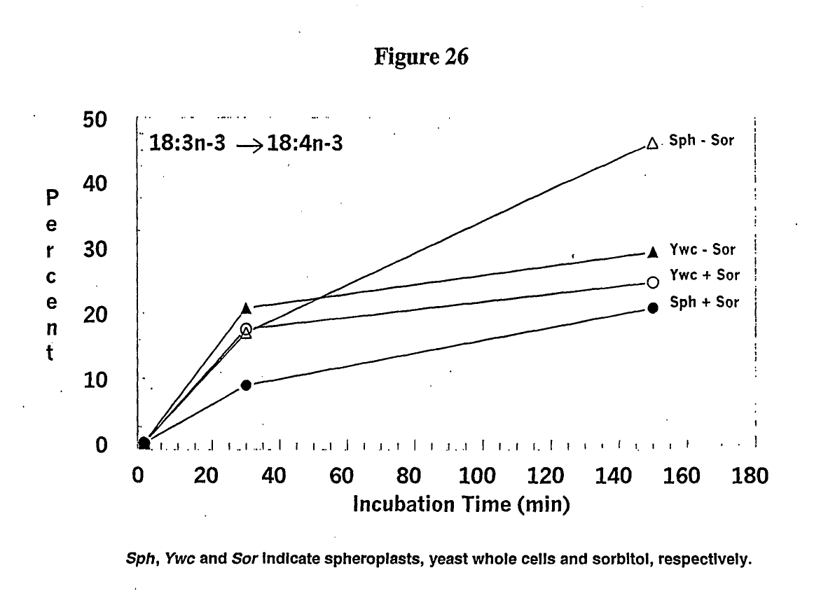

In spheroplasts incubated in SC-U medium without sorbitol, the radioactivity recovered from [1-¹⁴C]-24:4n-6 (2.05%) was similar to that from [1-¹⁴C]-alpha-linolenic (1.51%, FIGS. 25 and 26), but significantly higher than the radioactivity recovered from [1-¹⁴]-24:4n-6 in either spheroplasts or whole cells incubated with sorbitol (FIG. 25). Conversely, the uptake of [1-¹⁴C]-24:4n-6 by spheroplasts grown in a medium without sorbitol was greatly decreased to that detected in spheroplasts incubated with [1-¹⁴C]-alpha-linolenic in the presence of sorbitol (37.02%, FIGS. 25 and 26).

FIG. 26 shows that after 150 min of incubation, the conversion of [1-¹⁴C]-alpha-linolenic acid to 18:4n-3 was 2.3-fold higher (46.10%) in spheroplasts incubated in a SC-U medium without sorbitol than that detected in spheroplasts grown in medium containing sorbitol (20.74%). The delta-6-desaturation of [1-¹⁴C]-alpha-linolenic acid in whole cells was slightly affected by the presence of sorbitol in the medium, but the values remained significantly lower (29.32 and 24.61%/o) than those found in spheroplasts incubated in SC-U medium without sorbitol.

Under the experimental conditions of this study, the desaturation of [1-¹⁴C]-24:4n-6 was not detected.

Conclusion:

The treatment with Lyticase on Saccharomyces cerevisiae transformed with pYr5003.1 produced spheroplasts which were more efficient at uptake of [1-¹⁴C]-alpha-linolenic acid (18:3n-3) and Δ^{9,12,15,18}[1-¹⁴C]-tetracosatetraenoic acid (24:4n-6) than the yeast whole cells. The uptake of both fatty acids was affected by the presence of sorbitol in the incubation medium.

The desaturation of [1-¹⁴C]-alpha-linolenic acid (18:3n-3) by spheroplasts of Saccharomyces cerevisiae transformed with pYr5003.1 was time dependent and affected by the addition of sorbitol in the medium.

Delta-6 desaturation of [1-¹⁴C]-24:4n-6 was not detected in either spheroplasts or whole cells transformed with pYr5003.1. This seems to indicate that this rat delta-6-desaturase is specific for the two substrates alpha-linolenic acid (18:3n-3) and linoleic acid (18:2n-6) but not for the substrate 24:4n-6. This may imply the existence of another rat delta-6-desaturase specific for 24:4n-6, and possibly, 24:5n-3.

### Example 12

### Detection of Rat and Human Desaturase Gene Products In Saccharomyces Cerevisiae

Yeast Strain Construction:

The genotype of INVSc1 is (Mata/Mata his3Δ1/his3Δ1leu2/leu2 trp1-289/trp1-289 ura3-52/ura3-52). After having transformed Saccharomyces cerevisiae with the desaturase gene constructs as previously described, the resulting strains were isogenic to the INVSc1 except for the presence of the desaturase construct, namely, pYr5003.1, pTr5004.1, pYh5001.2 or pTh5002.1.

Growth and Induction of Expression:

Cloning in pYES2 results in the gene being expressed mostly in its native protein form. This is contrary to pYES2/CT where the gene of interest is expressed as a tagged protein with the V5/6×His epitope tags. The reason for working with the two vectors is to study function in the native form of the protein, and to monitor its expression by western blot using commercially available antibody against the V5 epitope (INVITROGEN).

Yeast cells were grown under selective pressure using synthetic complete medium lacking uracil (SC-uracil+2% raffinose) at 30° C. in incubator shaker using standard procedure (INVITROGEN). A 4 ml overnight pre-culture of each of the transformed yeast strains was prepared, and aliquots taken to inoculate a larger volume used for each experiment. On reaching OD₆₀₀=0.4±1.0, cells were divided and harvested at 3000 rpm for 5 minutes. One part was stored frozen and used as the zero induction time, and the second part was resuspended in SC-uracil+2% galactose and incubated at 30° C. in a shaker. The galactose will activate the GAL1 promoter to induce expression of the cloned gene. A time course for galactose induction of the cloned gene was assessed after 2, 4, 6 and 8 hrs by removing aliquots from the growing cells, harvesting and storing them.

Protein extraction was then performed on the samples using cell breaking buffer (50 mM sodium phosphate pH 7.4, 1 mM EDTA, 5% glycerol, 1 mM PMSF) as described by INVITROGEN, with slight modifications. The cells were induced to form spheroplasts by treating them with the cell wall digesting enzyme, lyticase (Sigma) at a final concentration of 2 units/ml in breaking buffer. Spheroplast formation was monitored microscopically. Cells were washed free of lyticase, harvested, weighed and resuspended in a corresponding volume of breaking buffer plus PMSF. About half volume of acid washed glass beads approximately 500 mm in diameter was added and cells were broken by vortexing 3× at 4° C. (30 sec vortex and 30 sec on ice). The crude protein extract was recovered at 3000 rpm for 3 minutes at 4° C. The crude extract was used for sodium dodecyl sulfate-polyacrylamide gel electrophoresis (SDS-PAGE) analysis and western blotting.

SDS-PAGE and Western Blotting:

Equal amounts of the crude protein extract was mixed with sample loading buffer (50 mM Tris pH 8, 2% SDS, 10 mM DTT, 0.1% bromophenol blue, 10% glycerol) and boiled at 100° C. for 5 minutes. The samples were loaded on 10% pre-cast SDS-polyacrylamide gels using standard procedures. Necessary molecular weight standards (Cruz Marker from Life Technologies) and controls were included. Protein samples were separated using electrophoresis buffer at a constant 100 V. After electrophoresis, the gel is either stained with Coomassie Blue to assess the presence of protein and as a loading control, or the protein is electrophoretically transferred onto a PVDF membrane (BIO-RAD). After the transfer, the membrane is blocked with a blocking solution and incubated with a 1:10,000 dilution of anti-V5-HRP antibody as described by the supplier (INVITROGEN). The membrane is washed and the antibody reaction detected with the Enhanced Chemi-Luminiscence reagent ECL (Amersham-Pharmacia Biotech). The membrane is exposed to Hyperfilm-ECL film (Amersham) in a cassette for 1-20 minutes. The film was developed and the signals scanned and quantified using the Gel Doc 2000 instrument (BioRad).

Results:

TABLE 19
Time course of protein expression of rat and human
delta-6-desaturases.

| Plasmid | 0 hours | 2 hours | 4 hours | 6 hours | 8 hours |
|---|---|---|---|---|---|
| pTr5004.1 | ND | ND | 4.5% | 15% | 100% |
| pTh5002.1 | ND | 3% | 3% | 49% | 100% |

ND: no protein detected with antibody
The band intensity at 8 hours was set to 100% for the purposes of this example.

Table 19 shows the relative time course over 8 hours for induction of the rD6D-1 and hD6D-1 tagged proteins in transformed yeast cells under galactose induction. The tagged enzymes are initially detected between 2 to 4 hours after induction and continue to accumulate throughout the course of the experiment.

### Example 13

### Screening Delta-6-Desaturase Modulators using Microsomes from Saccharomyces Cerevisiae Containing Human or Mammalian Desaturases

Yeast Microsome Preparation:

Two to 5 1 of Saccharomyces cerevisiae transformed with pYh5001.2 or pTh5002.1 are started with a cell density of approximately 3.2×10⁵cells/ml (O.D.₆₀₀0.4) using SC-U medium. After 8 h of incubation at 30° C. in an orbital incubator at 270 rpm, galactose is added to a final concentration of 2%. Yeast are further incubated for 12 h, harvested by centrifugation at 2060×g for 10 minutes at 4° C. and washed with water. The cell pellet is resuspended in ⅓ of its volume in isolation buffer containing 80 mM Hepes-KOH (pH 7.2) and 10 mM KCl and 320 mM of sucrose, 2 mM phenylmethylsulfonyl fluoride and an EDTA-free tablet of protease inhibitor cocktail (one tablet per 10 g cell pellet; Complete, Roche, Germany). The cell suspension is poured into a mortar containing liquid N₂ and ground with sand using a ceramic pestle. The yeast powder is transferred to a conical test tube, to which isolation buffer is added (⅔ of the pellet volume). The sand is removed by centrifugation at 57×g for 1 min and the suspension is further centrifuged at 10,000×g for 20 min to separate cell debris, nuclei, and mitochondria. The supernatant is then centrifuged at 106,000×g for 1 h to obtain the microsome pellet which is resuspended in 700 μl of isolation buffer. The protein concentration of the microsome suspension is measured by any technique known in the art.

Incubation of Delta-6-Desaturase Modulators with Yeast Microsomes:

The activity of delta-6-desaturase is determined by measuring the conversion of [1-¹⁴C]20:3n-6 (dihomogammalinolenic acid) to [1-¹⁴C]20:4-6 (arachidonic acid). Reactions are started by adding 500 μg of yeast microsomal protein, to pre-incubated tubes containing 0.20 μCi of the substrate fatty acid at a final concentration of 33 μM in 0.25 ml of the incubation solution, containing 80 mM Hepes-KOH (pH 7.2) and 43.2 mM MgCl₂, ATP (1.0 mM), NADH (500 μM) and coenzyme A (10 μM) and a range of concentrations of the enzyme modulators. The tubes are vortexed vigorously and after 15 min incubation in a shaking water bath (37° C.), the reactions are stopped by the addition of 2 ml of 10% (w/v) KOH in ethanol. Lipids in the incubation mixture are saponified at 80° C. for 45 min under N₂. The samples are left in ice for 5 min and then acidified with HCl. The fatty acids are extracted with hexane and esterified with BF₃/methanol at 90° C. for 30 min. The fatty acid methyl esters, substrate and product of the enzymatic reaction, are analyzed by HPLC as described above. Results are expressed in pmol of arachidonic acid produced per mg microsomal protein per minute. Alternatively, fatty acid methyl esters are analyzed by capillary column gas chromatography (GC).

### Example 14

### Screening Delta-6-Desaturase Modulators using Purified Enzymes Obtained from Saccharomyces Cerevisiae Expressing Human or Mammalian Desaturases

Isolation of the delta-6-desaturase from yeast microsomes:

Yeast microsomes containing delta-6-desaturase tagged with 6xHis are stirred with Zwittergent 3-14 or mixtures of deoxycholate/Triton X-100 (2%, w/w) for 2 h at 4° C. to solubilize the delta-6-desaturase. Alternatively, yeast microsomes can be treated with 2.5% (v/v) water in acetone to improve the solubilzing power of the detergents. The mixture is centrifuged at 106,000×g for 1 h The supernatant containing the enzyme is loaded onto a pre-equilibrated HiTrap chelating (Ni⁺⁺ charged iminodiacetate) column (Pharmacia) attached to a fast protein liquid chromatography system (Pharmacia). The column is washed with 50 mM sodium phosphate (pH 8.0). The tagged protein is eluted with sodium phosphate buffer containing imidazole (0-500 mM) and concentrated by ultrafiltration using Centriprep (Amicon, Mass.) concentrators.

### Incubation of Delta-6-Desaturase Modulators with Purified Enzyme:

The concentrated enzyme is incubated at 30-37° C. in Tris-HCl buffer (pH 7.2) containing 1 mM NADH, 80 μM of cytochrome b₅, 4 μM of NADH-cytochrome b₅ reductase, 6 mM egg phosphatidylcholine, 2% Triton X-100, 0.4% sodium deoxycholate, radiolabelled dihomogammalinolenyl-CoA as the enzyme substrate and a range of concentrations of each enzyme modulator. After 15-90 min of incubation, the reaction is stopped and fatty acids, substrate and product of the enzymatic reaction, are analyzed as described in herein.

Alternatively, the enzyme activity and the effect of modulators of the enzyme activity can be measured by the rate of NADH oxidation in the presence and absence of dihomogammalinolenyl-CoA.

### Example 15

### Validation of Assays Described in Examples above using Rat Liver Microsomes

Preparation Rat Liver Microsomes:

Wistar rats under light halothane (15% in mineral oil) anesthesia were sacrificed by exsanguination during periods of high enzyme activity. Livers are immediately rinsed with cold 0.9% NaCl solution, weighed and minced with scissors. All procedures are performed at 4° C. unless specified otherwise. Livers are homogenized in a solution (1:3 w/v) containing 0.25 M sucrose, 62 mM potassium phosphate buffer (pH 7.0), 0.15 M KCl, 1.5 mM N-acetylcysteine, 5 mM MgCl₂, and 0.1 mM EDTA using 4 strokes of a Potter-Elvehjem tissue homogenizer. The homogenate is centrifuged at 10,400×g for 20 min to eliminate mitochondria and cellular debris. The supernatant is filtered through a 3-layer cheesecloth and centrifuged at 105,000×g for 60 min. The microsomal pellet is gently resuspended in the same homogenization solution with a small glass/teflon homogenizer and stored at −70° C. The absence of mitochondrial contamination is enzymatically assessed. The protein concentration is measured using bovine serum albumin as the standard.

Incubation of Rat Liver Microsomes with Delta-6-Desaturase Modulators:

Reactions are started by adding 2 mg of microsomal protein to pre-incubated tubes containing 0.20 μCi of the substrate fatty acid (DGLA) at a final concentration of 33.3 μM in 1.5 ml of homogenization solution, containing NaF (42 mM), niacinamide (0.33 mM), ATP (1.57 mM), NADH (1.0 mM), coenzyme A (0.09 mM) and a range of concentrations of the enzyme modulators. N-propyl gallate was added to the incubation medium to a final concentration of 0.02-0.32 mM. The tubes are vortexed vigorously and after 15 min incubation in a shaking water bath (37° C.), the reactions are stopped and fatty acids are analyzed as described in herein. Alternatively, fatty acid methyl esters are analyzed by capillary column gas chromatography (GC).

Table 20 shows the in vitro inhibition of delta-6-desaturase with different concentrations of n-propyl gallate in rat liver microsomes. A plateau was reached at concentration of the inhibitor that ranged between 0.08-0.32 mM.

TABLE 20
Inhibition of delta-6-desaturase activity in rat liver microsomes incubated
with [1-¹⁴C]-dihomogammalinolenic acid and n-propyl gallate.

| Inhibitor conc. (mM) | % of inhibition |
|---|---|
| 0.00 | 0.0 |
| 0.02 | 62.9 |
| 0.04 | 74.9 |
| 0.08 | 86.4 |
| 0.16 | 88.7 |
| 0.32 | 100.0 |

Values are the mean of three determinations. Enzyme activity without inhibitor: 394 pmol/mg microsomal protein/min.

### Example 16

### Screening of Delta-6 and Delta-5-Desaturase Modulators Using Saccharomyces Cerevisiae Whole Cells, Spheroplasts or Microsomes Containing Human or Mammalian Delta-6 and Delta-5-Desaturases

This method is suitable for simultaneous drug screenings of both fatty acid desaturases under the same experimental conditions. The specificity of each drug for each enzyme is rapidly determined by this method.

Co-Expression of Human or Mammalian Delta-6 and Delta-5-Desaturases in Yeast:

Delta-6 and delta-5-desaturase genes are cloned in 2 separate yeast vectors (constitutive or inducible), having different nutritional selection markers, for examples, URA3 and LEU2 genes which confer uracil and leucine prototrophy for selection in yeast. A yeast strain having an auxotrophic requirement for uracil and leucine is transformed with the two plasmids. Yeast cells containing the plasmids are selected on synthetic minimal medium lacking both uracil and leucine. The activity of the two desaturases is then assayed and used for screening of modulators.

Alternatively, bi-directional yeast vectors, for example the pBEVY plasmids (Miller et al., 1998, Nucl. Acid Res. 26:3577-3583), are used to co-express the desaturase genes. The pBEVY plasmids provide for either constitutive or galactose-induced expression of exogenous genes.

The fatty acid desaturase genes are cloned downstream of the constitutive glyeraldehyde-3-phosphate dehydrogenase (GPD) and the alcohol dehydrogenase 1 (ADH1) promoters, respectively, using methods known to those skilled in the art. Alternatively, the genes are cloned on both sides of the bi-directional galactose inducible promoter GAL1/GAL10. A suitable yeast strain (auxotrophic for a nutritional requirement, e.g. uracil) is transformed with desaturase constructs (for example, which confer uracil prototrophy). Such yeast transformants are selected in SC-U medium. The selected transformants are grown in appropriate media to allow constitutive or inducible expression of the two proteins.

The present method utilizes bi-directional vectors expressing mammalian fatty acid delta-6- and delta-5-desaturases to screen simultaneously for unique modulators of both or either activities that may have therapeutic, diagnostic or nutritional function.

Whole Yeasts or Spheroplasts:

The enzymatic assay with modulators of both enzymes is similar to that described above (Example 15). In this model, the radiolabelled substrates for delta-6 and delta-5-desatuases, alpha-linolenic (18:3n-3) and DGLA (20:3n-6) acids, respectively, are both added in the incubation medium. After 2-19 h of incubation the remnant radiolabelled substrates and products (stearidonic acid, 18:4n-3, and arachidonic acid, 20:4n-6, respectively) of the enzymatic reaction are analyzed by HPLC as described herein. Alternatively, fatty acid methyl esters are analyzed by capillary column gas chromatography (GC).

Microsomes:

Microsomes from yeast containing both human delta-6- and delta-5-desaturases or both mammalian (e.g. rat) delta-6- and delta-5-desaturases are obtained as previously described (Example 13). The incubation is similar to that used with microsomes containing only one human or mammalian desaturase with the exception that radiolabelled alpha-linolenic acid (18:3n-3) and DGLA (20:3n-6), substrates for delta-6 and delta-5-desaturases, respectively, are both added to the incubation medium along with a range of different concentrations of desaturase modulators. The products of the enzymatic reaction are analyzed by HPLC as described herein. Alternatively, fatty acid methyl esters are analyzed by capillary column gas chromatography (GC).

### EXAMPLE 17

### Inhibition of Delta-6-Desaturase Activity in Saccharomyces Cerevisiae Whole Cells and Spheroplasts Transformed With Rat or Human Desaturase Genes

Introduction:

Several compounds are known to inhibit fatty acid delta-5- and delta-6-desaturases, in rat liver microsomes, microalgae or fungi. Early reports have shown the inhibitory effects of the positional isomers of trans-18:1 acids on desaturase activity (Mahfouz et al., 1980, Lipids, 15:100-107). More recently Kawashima et al., 1996, Biosci. Biotech. Biochem., 60:1672-1676 have described different types of inhibitors: (1) lignan compounds of sesame seeds or oils (sesamin, episesamin, sesaminol and sesamolin as specific inhibitors for delta-5-desaturase), (2) alkyl gallate, (3) diferuloyl methane (curcumin) and (4) nicardipine and nifedipine (Ca⁺⁺ channel blockers and anti-hypertension drugs). Other compounds such as salicylhydroxamic acid (Khozin-Goldberg et al., 1999, Biochim. Biophys. Acta, 1439: 384-394) or aniline derivatives (Obukowicz et al., 1998, Biochem. Pharmacol., 55:1045-1051), have been also used to inhibit fatty acid desaturases in microorganisms or rat microsomes.

This example demonstrates the use of a novel model (i.e. transformed yeast with rat or human fatty acid delta-6-desaturase genes) for inhibitor (or enhancer) screening of mammalian desaturases. Rat liver microsomes were used to corroborate the assays.

The propyl ester of 3,4,5-trihydoxybenzoic acid (n-propyl gallate), commonly used as antioxidant in the food industry (eg. for fats and oils) was selected for this example due to its substantial inhibitory effects on both delta-5- and delta-6-desaturases and its high solubility in water or ethanol. It has been reported that n-propyl gallate is a noncompetitive inhibitor of fatty acid desaturases (Kawashima et al., 1996, Biosci. Biotech. Biochem., 60: 1672-1676).

Chemicals and Radiochemicals:

Propyl ester of 3,4,5-trihydroxybenzoic acid (n-propyl gallate), tergitol, tris buffer, fatty acid free bovine serum albumin, carbohydrates, sorbitol, amino acids, fatty acids, Lyticase and DDT (dithiothreitol) were obtained from Sigma-Aldrich Canada (Qakville, ON, Canada). Yeast nitrogen base without amino acids was purchased from Difco (Becton Dickinson Co; Sparks, Md., U.S.A.). All organic solvents (HPLC grade) were obtained from Fisher-Scientific (Fair Lawn, N.J., U.S.A.).

[1-¹⁴C]-alpha-linolenic acid (18:3n-3; 99% radiochemical purity; specific activity: 52 μCi/μmol) and [1- ¹⁴C]-linoleic acid (18:2n-6; 99% radiochemical purity; specific activity: 51 μCi/μmol) were purchased from NEN (Boston, Mass., U.S.A.). These fatty acids were saponified with KOH (0.1 M) and dissolved in modified SC-U medium (minimum medium containing 1% raffinose but lacking uracil) with 1% tergitol.

Rat Liver Microsome Preparation:

Hepatic microsomes were obtained from female Wistar rats by differential centrifigation as described in Example 3.

Spheroplast Preparation:

Saccharomyces cerevisiae transformed with pYr5003.1 without V5/6×His tags or pTh5002.1 with V5/6×His tags were grown in modified SC-U medium with 2% galactose to induce the expression of the gene that encodes the fatty acid delta-6-desaturase. After 16 h incubation, cells were centrifuged at 2060×g for 5 min at 4° C., washed once with distilled water and centrifuged again. The volume and weight of the cell pellet were measured Cells were suspended (1:2 w/v) in 0.1 M Tris.SO₄ (pH 9.4), 10 mM DTT and incubated at 30° C. After 10 min incubation, the cell pellet was obtained by centrifugation, washed once (1:20 w/v) with 1.2 M sorbitol and suspended (1:1 w/v) in 1.2 M sorbitol, 20 mM phosphate buffer (pH 7.4) as described elsewhere (Daum et al., 1982, J. Biol. Chem., 257: 13028-13033). The 15,800× g (1 min) supernatant of Lyticase was added to the cell suspension at a concentration of 2000 U/ml and the suspension incubated at 30° C. with 50 rpm shaking. Conversion to spheroplasts was checked after 40 min incubation by diluting the suspension with distilled water followed by observation under the microscope (Schatz G. and Kovac L., 1974, Methods in Enzymology, 31A: 627-632). After 70 min incubation, approximately 90% of the cells were converted to spheroplasts.

Spheroplasts Incubation:

Spheroplasts were harvested by centrifugation at 2060×g for 5 min at 4° C. and washed once with 1.2 M sorbitol. Spheroplasts were suspended in modified SC-U medium with 1% tergitol, 1.2 M sorbitol and 2% galactose to maintain the induction conditions and to give an OD₆₀₀ reading of approximately 2.5-3.0. A 10 ml aliquot of the spheroplast suspension was transferred to a 125 ml Erlenmeyer flask and incubated with 200 μL of n-propyl gallate in ethanol (final concentration in the culture ranged between 0.7 and 14.1 mM) at 30° C. in an orbital incubator set at 270 rpm After 30 min incubation 1 μCi of [1-¹⁴C]-alpha-linolenic or [1-¹⁴C]-linoleic acids were added to the cultures to a final concentration of 2 μM and further incubated for 120 min. At this time point, turbidity readings at OD₆₀₀ were taken, spheroplasts were harvested by centrifugation and washed with Tris buffer (100 mM, pH 8.0) containing 0.1%/o BSA. Total lipids were extracted as described below.

Whole Yeast Incubations:

Transformed Saccharomyces cerevisiae pTh5002.1 with V5/6xHis tags were incubated in a 125 ml Erlenmeyer flask containing 9 ml of modified SC-U medium with 1% tergitol (O.D.₆₀₀0.4, approximately 3.2×10⁶ cells) and 200 μl of 7, 14 or 28 mM n-propyl gallate in ethanol. After 1 h incubation in an orbital incubator at 270 rpm and 30° C., 1 μCi of potassium salts of [1-¹⁴C]-linoleic or [1-¹⁴C]-alpha-linolenic acids (dissolved in modified SC-U medium and 1% tergitol) were added to the cell suspension to a final concentration of 2 μM. After 5 h incubation with the inhibitor, cells reached the log phase and the transgene expression was induced with the addition of 1 ml of galactose to a final concentration of 2%. Yeast were further incubated for 19 h (OD₆₀₀ range 7.2-9.3) until they were harvested by centrifugation at 5000×g for 10 minutes at 4° C. Cell were washed with Tris buffer (100 mM, pH 8.0) containing 0.1% BSA and total lipids were extracted as described below.

Rat Liver Microsome Incubation:

Incubations were performed as described in Example 3. N-propyl gallate was added to the incubation medium to a final concentration that ranged from 0.02 to 0.32 mM.

Lipid Extraction:

The radioactivity from aliquots of the supernatant, spheroplasts and whole cells was determined by liquid scintillation counting using a LS6500-Scintillation System (Beckman) prior to the lipid extraction. Total lipids were extracted from cells or spheroplasts with chloroform/methanol (2:1 v/v) according to the method of Folch et al., 1957, J. Biol. Chem., 226: 497-509. The total lipid extracts were methylated using boron trifluoride in methanol at 90° C. for 30 min. The resultant methyl esters (FAME) were analyzed as previously described in Example 3.

Calculations:

The desaturase activity was determined by measuring the conversion of radiolabelled linoleic acid to gammna-linolenic acid (18:2n-6 to 18:3n-6) and alpha-linolenic acid to stearidonic acid (18:3n-3 to 18:4n-3). The percent inhibition was calculated as described elsewhere (Kawashima et al., 1996, Biosci. Biotech. Biochem., 60:1672-1676):

Inhibition % =(activity without the inhibitor−activity with the inhibitor)/activity without the inhibitor

Results:

N-propyl gallate inhibited the conversion of substrates of fatty acid delta-6-desaturase in spheroplasts from Saccharomyces cerevisiae transformed with human or rat genes that encode the enzyme (Table 21 and 22). The constant O.D.₆₀₀ readings (i.e. constant number of cells) and the similar levels of radioactivity recovered in cells at concentrations of n-propyl gallate between 0.7-5.6 mM indicate that the inhibitor was not affecting the uptake of substrate and that it was not cytotoxic. However, at concentrations >5.6 mM, the cell number was slightly decreased and the radioactivity recovered in those cells from the fatty acid substrates was substantially reduced demonstrating that those concentrations may be toxic for the spheroplasts.

TABLE 21
Inhibition of hD6D-1 (with V5/6xHis tags) in spheroplasts of
transformed Saccharomyces cerevisiae incubated with
[1-¹⁴C]-alpha-linolenic acid and n-propyl gallate.

| Inhibitor | % of | | % of [1-¹⁴C]-ALA |
|---|---|---|---|
| Concentration (mM) | inhibition | O.D.₆₀₀ | Recovered in Cells |
| 0.0 | 0 | 3.3 | 42.6 |
| 0.7 | 11.6 | 2.2 | 46.5 |
| 1.4 | 18.8 | 3.4 | 36.3 |
| 2.8 | 56.4 | 3.1 | 39.0 |
| 5.6 | 82.3 | 3.5 | 34.9 |
| 14.1 | 100 | 2.5 | 17.6 |

Values are the mean of three determinations. The desaturase activity was assessed by measuring the conversion of alpha-linoleic acid to stearidonic acid (18:n-3 tp 18:n-3).

TABLE 22
Inhibition of rD6D-1 (without V5/6xHis tags) in spheroplasts
of transformed Saccharomyces cerevisiae incubated with
[1-¹⁴C]-linoleic acid and n-propyl gallate.

| Inhibitor | % of | | % of [1-¹⁴C]-LA |
|---|---|---|---|
| Concentration (mM) | inhibition | O.D.₆₀₀ | Recovered in Cells |
| 0.0 | 0 | 2.5 | 16.9 |
| 0.7 | 8.9 | 2.5 | 13.6 |
| 1.4 | 39.2 | 2.6 | 16.7 |
| 2.8 | 77.4 | 2.5 | 16.1 |
| 5.6 | 100 | 2.6 | 14.8 |
| 14.1 | 100 | 2.3 | 5.1 |

Values are the mean of three determinations. The desaturase activity was assessed by measuring the conversion of linolenic acid to gamma-linolenic acid (18:3n-3 to 18:4n-3).

The induction of desaturase gene expression for 16 h prior to the addition of the inhibitor guaranteed that the observed reduction in substrate conversion was not due to an inhibition of transcription or translation for the genes.

The inhibitory effect of n-propyl gallate was also detected in whole yeast in which the desaturase gene was induced after the addition of the inhibitor (Table 23).

TABLE 23
Inhibition of hD6D-1 (with V5/6xHis tags) in whole cells
of transformed Saccharomyces cerevisiae incubated with
[1-¹⁴C]-alpha-linolenic acid and n-pr pyl gallate.

| Inhibitor | % of | | % of [1-¹⁴C]-ALA |
|---|---|---|---|
| Concentration (mM) | inhibition | O.D.₆₀₀ | Recovered in Cells |
| 0 | 0 | 8.6 | 1.7 |
| 7 | 36.7 | 9.3 | 2.3 |
| 14 | 73.6 | 7.2 | 2.4 |
| 28 | — | 0 | — |

Values are the mean of three determinations. The desaturase activity was assessed by measuring the conversion of alpha-linolenic acid to stearidonic acid (18:3n-3 to 18:4n-3).

Table 24 shows the inhibition of delta-6-desaturase in rat liver microsomes. The conversion of both fatty acid substrates, linoleic and alpha-linolenic acids, was inhibited in vitro by different concentrations of n-propyl gallate. A plateau was reached between 0.16 and 0.32 mM.

TABLE 24
Inhibition of delta 6 desaturase activity in rat liver microsomes incubated
with [1-¹⁴C]-alpha-linolenic or [1-¹⁴C]-linoleic acids and n-propyl gallate.

| | |
|---|---|
| Inhibitor | % of inhibition |

| Concentration (mM) | 18:2n-6 → 18:3n-6 | 18:3n-3 → 18:4n-3 |
|---|---|---|
| 0.00 | 0 | 0 |
| 0.02 | 18.0 | 28.7 |
| 0.04 | 32.0 | 42.5 |
| 0.08 | 64.5 | 54.2 |
| 0.16 | 82.6 | 71.3 |
| 0.32 | 98.7 | 79.5 |

Values are the mean of three determinations. The desaturase activity was assessed by measuring the conversion of linoleic acid to gamma-linolenic acid (18:2n-6 to 18:3n-6) and alpha-linolenic acid to stearidonic acid (18:3n-3 to 18:4n-3).

Conclusions:

Spheroplasts transformed with fatty acid desaturase genes should be considered as the model of choice for desaturase assays since lower concentrations of inhibitors (than those used with the whole yeast) are required to obtain detectable changes in the enzyme activity. Therefore, in this model, the solubility restrictions of the inhibitors are reduced. In addition, in spheroplasts, due to the lack of an intact cell wall, the uptake of the desaturase substrates is higher than in whole yeast which helps to increase the threshold of detection (Tables 21 and 23). These assays should be performed using concentrations of the inhibitors below the cytotoxic levels.

The strength of this novel model for drug screening using mammalian desaturase genes in yeast is supported by the results obtained using the traditional method of rat liver microsomes.

In summary, spheroplasts from Saccharomyces cerevisiae transformed with mammalian fatty acid desaturase genes are useful for screening inhibitors and enhancers of delta-6-desaturase. The cell wall mutant yeast allow a greater flux of fatty acids to enter the yeast cell which is beneficial in determining D6D, D5D or elongase activity in a dual enzyme assay, for example.

### Reference List

U.S. Pat. No. 4,806,569, February, 1989, Horrobin D. F.

U.S. Pat. No. 4,826,877, May 2, 1989, Stewart et al.

International Patent Application No. WO 88/04300, June, 1988, Cech et al.

Patent Cooperation Treaty International Publication No., WO 93/05182, March, 1993, Bruice

Actis Dato et al., 1973, Lipids, 8: 1-6

Aki et al., 1999, Biochem. Biophys. Res. Commun., 255: 575-579

Altschul et al., 1990, J. Mol. Biol., 215: 403-410

Altschul et al., 1997, Nucleic Acids Res, 25: 3389-3402

Anderson et al., 1998, Anticancer Res., 18: 791-800

Arisaka et al., 1986, J. Paediatr. Gastroenterol. Nutr., 5: 878-882

Ausubel et al., 1994-, Current Protocols in Molecular Biology, John Wiley & Sons, New York, N.Y

Been M. D. and Cech T. R., 1986, Cell, 47: 207-216

Begin et al., 1986, J. Nat. Cancer Inst., 77: 1053-1062

Betteridge D. J.,1999, Eur. J. Clin. Invest., Vol. June 29, Suppl. 2: 12-6

Biagi et al., 1991, Biochimica et Biophysica Acta, 1083: 187-192

Brenner et al., 1968, Am. J. Physiol., 215: 63-70

Calviello et al., 1998, Int. J. Cancer, 75: 699-705

Carillo H. and Lipman D., 1988, SIAM J.Applied Math., 48:1073

Chapman & Hall ed., 1992, Unsaturated Fatty Acids: Nutritional and Physiological Significance,

British Nutrition Foundation, London, pp. 175

Cho et al., 1999a, J. Biol. Chem., 274: 471-477

Cho et al., 1999b, J. Biol. Chem., 274: 37335-37339

Cook H. W., 1996, Fatty Acid Desaturation and Chain Elongation in Eukaryotes: in Biochemistry of Lipids, Lipoproteins and Membranes, Vance D. E. and Vance J. E. (eds.) Elsevier, Amsterdam, pp.150

Copsey D. N. and Delnatte S. Y. J., 1988, Genetically Engineered Human Therapeutic Drugs, Stockton Press, New York

Coste et al., 1999, J. Nutr. Biochem., 10: 411-420

Cotter M. A and Cameron N. E., 1997, Diabetic Neuropathy, Marius Press, Camforth, U. K., pp. 97-119

Dang et al., 1989, Lipids, 24: 882-889

Daum et al., 1982, J. Biol. Chem., 257: 13028-13033

de Antueno et al., 1993, Lipids, 28: 285-290

de Antueno et al., 1994, Lipids, 29: 327-331

Deutcher, M. (editor), 1990, Guide to Protein Purification. Meth. Enzymol.: 182

Devereux et al., 1984, Nucl. Acid Res., 12: 387-395

Dines et al., 1993, Diabetologia, 30: 1132-1138

du Toit et al., 1994, Prostaglandins Leukot. Essent. Fatty Acids, 51: 121-124

Faas F. H. and Carter W. J., 1980, Lipids, 15: 953-961

Faas F. H. and Carter W. J., 1983, Lipids, 18: 339-342

Falconer et al., 1994, Br. J. Cancer, 69: 826-832

Folch et al., 1957, J. Biol. Chem., 226: 497-509

Giron et al., 1999, Metabolism, 48: 455-460

Gribskov M. and Devereux J., eds., 1991, Sequence Analysis Primer, M Stockton Press, NY

Griffin A. M. and Griffin H. G., eds., 1994, Computer Analysis of Sequence Data, Part 1, Humana Press, NJ

Guthrie C. and Fink G., 1991, Meth. Enzymol., 194

Hanahan et al., 1983, J. Mol. Biol., 166: 557-580

Hansen A. E., 1933, Proc. Soc. Exp. Biol. Med. 31: 160-161

Haseloff J. and Gerlach W. L., 1988, Nature, 334: 585-591

Herbst et al., 1994, Gene, 151: 137-142

Horrobin D. F., 1990, Rev. Contemp. Pharmacother., 1: 1-45

Horrobin D. F., 1992, Prog. Lipid Res., 31: 163-194

Hrelia et al., 1989, Biochem. Biophys. Res. Comm., 163: 348-355

Igal et al., 1991, Mol Cell Endocrinol., 77: 217-227

Inkpen et al., 1969, J. Lipid Res., 10: 277-282

Izant J. G. and Weintraub H., 1984, Cell, 36: 1007-1015

Janknecht et al., 1991, Proc. Natl. Acad Sci, USA, 88: 8972-8976

Jiang et al, 1995a, Br. J. Cancer, 71: 5043-5048

Jiang et al., 1995b, Br. J. Cancer, 71: 744-752

Jiang et al., 1997, Prostaglandins Leukot. Essent. Fatty Acids, 56: 307-316

Jiang et al., 1998a, Br. J. Cancer, 77: 731-738

Jiang et al., 1998b, Biochem. Biophys. Res. Commun., 244: 414-420

Johnston M., 1987, Microbiol. Rev., 51: 458-476

Julu P., 1997, in Essential Fatty Acids and Eicosanoids, pp.168-175

Kawashima et al., 1996, Biosci. Biotech. Biochem., 60: 1672-1676

Kawashima Y. and Kozuka H., 1985, Biochimica et Biophysica Acta, 834: 118-123

Keen et al. 1993, Diabetes Care, 16: 8-15

Khozin-Goldberg et al., 1999, Biochim. Biophys. Acta, 1439: 384-394

Kilberg M. S. and Christensen H. N., 1979, Biochemistry, 18: 1525-1530

Kreisberg R. A., 1998, Am. J Cardiol., 82: 67U-73U discussion 85U-86U

Kuruvilla et al., 1998, Prostaglandins Leukot. Essent. Fatty Acids, 59: 196-202

Lai et al., 1996, Br. J. Cancer, 74: 1375-1383

Leikin A. I. and Brenner R. R, 1987, Biochim. Biophys. Acta, 922: 294-303

Leildn A. I. and Brenner R. R, 1989, Biochim. Biophys. Acta. September 25, 1005: 187-191

Lesk A. M., ed., 1988, Computational Molecular Biology, Oxford University Press, NY

Llewellyn et al., 1987, J. Mol. Biol., 195: 115-123

Lowry et al., 1951, J. Biol. Chem., 193: 265-275

Lue et al., 1987, Mol. Cell. Biol., 7: 3446-3451

Mack E. W., 1990, Remington's Pharmaceutical Sciences, Mack Publishing Company, Easton, Pa., 13^{th} edition

Mahfouz et al., 1980, Lipids, 15: 100-107

Manku et al., 1984, Br. J. Dermatol., 110: 643-680

Margolskee, et al., 1988, Mol. Cell. Biol. 8: 2837-2847

McLaughlin et al., 1988, J. Virol. 62: 1963-1973

Miller et al, 1998, Nucl. Acid Res. 26:3577-3583

Mimouni V. and Poisson J. P., 1991, Archives internationales de Physiologie et de Biochimie, 99: 111-121

Mimouni V. and Poisson J. P., 1992, Biochim. Biophys. Acta, 1123: 296-302

Morrison W. R. and Smith L. M., 1964, J. Lipid Res., 5: 600-608

Moss et al., 1987, Annu. Rev. Immunol. 5: 305-324

Nishi et al., 2000, Biochim. Biophys. Acta., 1490: 106-108

Obukowicz et al., 1998, Biochem. Pharmacol., 55:1045-1051

Okano et al., 1988, EMBO J., 7: 3407-3412

Old R. W. and Primrose S. B., 1994, Principles of Gene Manipulation, 5^{th} Edition, Blackwell Scientific Publications, U.S.A

Orkin, et al., 1988, Prog. Med Genet. 7: 130-142

Persson et al., 1996, Scand. J. Clin. Lab. Invest., 56: 183-190

Plumb et al., 1993, Br. J. Cancer, 67: 728-733

Rasmussen, et al., 1987, Meth. Enzymol. 139: 642-654

Rosenberg et al., 1985, Nature, 313: 703-706

Sambrook et al., 1989, Molecular Cloning, A Laboratory Manual, 2^{nd} edition, Cold Spring Harbour Press, Cold Spring Harbour, N.Y.

Saitou N and Nei M., 1987, Mol. Biol. Evol., 4: 406-425

Schatz G. and Kovac L., 1974, Methods in Enzymology, 31A: 627-632

Seegers et al., 1997, Prostaglandins Leukot Essent. Fatty Acids, 56: 271-280

Sinclair H. M., 1984, Hum. Nutr. Clin. Nutr., 38: 245-260

Smith D. W., ed., 1993, Biocomputing: Informatics and Genome Project, Academic Press, NY

Sonnhammer et al., 1998, in Proc of Sixth Int Conf on Intelligent Systems for Molecular Biology, AAAI Press, Menlo Park, CA pp. 175-182

Takeda et al., 1993, Anticancer Res., 13:193-200

Thompson et al., 1994, Nucleic Acid Res., 22: 4673-4680

Tilvis R. S. and Miettinen T. A, 1985, J. Clin. Endocrinol. Metab., 61: 741-745

Todd et al., 1999, Plant J., 17: 119-130

Tomlinson D. R, 1998, Diabetes and Metabolism (Paris) , 24 (suppl) 3: 79-83

Ulman et al., 1991, Lipids: 26: 127-133

Van Doormaal et al., 1988, Diabetologia, 31: 576-584

Van Roermund et al., 1998, EMBO J., 17: 677-687

Vartak et al., 1997, Br. J. Cancer, 77: 1612-1620

von Heijne G., 1987, Sequence Analysis in Molecular Biology, Academic Press, NY

Water K. M., 1997, Biochim. Biophys. Acta., 1349: 33-42

Weber et al., 1994, J. Nat. Cancer Inst., 86: 638-639

Wigmore et al., 1997, Clin. Sci., 92: 215-221

Wright et al., 1982, Lancet, 2: 1120-1122

Zaug A. J. and Cech T. R, 1986, Science, 231: 470-475

Zaug et al., 1984, Science, 224: 574-578

Zaug, et al., 1986, Nature, 324: 429-433

We claim:

1. An isolated polynucleotide segment, comprising a polynucleotide sequence which is selected from the group consisting of:
(a) a sequence comprising SEQ ID NO: 1;
(b) a sequence comprising SEQ ID NO: 2;
(c) a sequence which is at least 80% homologous with a sequence of any of (a) to (b);
(d) a sequence which is at least 90% homologous with a sequence of any of (a) to (b);
(e) a sequence which is at least 95% homologous with a sequence of any of (a) to (b);
(f) a sequence which is at least 98% homologous with a sequence of any of (a) to (b);
(g) a sequence which is at least 99% homologous with a sequence of any of (a) to (b); and;
(h) a sequence which hybridizes to any of (a) to (g) under stringent conditions.

2. An isolated polynucleotide segment of claim 1, wherein the isolated polynucleotide segment is genomic DNA.

3. A vector comprising a polynucleotide segment of claim 1 in a suitable vector.

4. A host cell comprising a polynucleotide segment of claim 1 in a host cell which is heterogeneous to said segment.

5. A method for producing a polypeptide encoded by a gene operably inked to a polynucleotide segment of claim 1 comprising the step of culturing the host cell of claim 4 under conditions sufficient for the production of said polypeptide.

6. An isolated polynucleotide fragment selected from the group consisting of:
(a) a sequence having at least 15 sequential bases of nucleotides of a segment of claim 1;
(b) a sequence having at least 30 sequential bases of nucleotides of a segment of claim 1; and
(c) a sequence having at least 50 sequential bases of nucleotides of a segment of claim 1.

7. A vector comprising a polynucleotide segment of claim 6 contained in a vector which is heterogeneous to said segment.

8. An isolated polynucleotide segment, comprising a polynucleotide sequence which retains substantially the same biological function or activity as the polynucleotide encoded by a segment of claim 1.

9. A method for identifying a compound which inhibits or promotes the activity of a polynucleotide segment of claim 1, comprising the steps of:
(a) selecting a control animal having said segment and a test animal having said segment;
(b) treating said test animal using a compound; and,
(c) determining the relative quantity of an expression product of an operably linked polynucleotide to said segment, as between said control animal and said test animal.

10. A method of claim 9, wherein said animals are mammals.

11. A method of claim 10, wherein said mammals are rats.

12. A method for identifying a compound which inhibits or promotes the activity of a polynucleotide segment of claim 1, comprising the steps of:
(a) selecting a host cell of claim 4;
(b) cloning said host cell and separating said clones into a test group and a control group;
(c) treating said test group using a compound; and
(d) determining the relative quantity of an expression product of a polynucleotide operably linked to said polynucleotide segment, as between said test group and said control group.

13. A method for identifying a compound which inhibits or promotes the activity of a polynucleotide segment of claim 1, comprising the steps of:
(a) selecting a test group having a host cell of claim 4, a part thereof or an isolated polynucleotide thereof and a control group;
(b) treating said test group using a compound; and
(c) determining the relative quantity of an expression product of an operably linked polynucleotide to said segment, as between said test group and said control group.

14. A composition for treating a lipid metabolism disorder comprising a compound which modulates a segment according to claim 1 and a pharmaceutically acceptable carrier.

15. A composition as claimed in claim 14, wherein said disorder is selected from the group consisting of atopic eczema, mastalgia, rheumatoid arthritis, Sjögren'syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

16. A composition as claimed in claim 15, wherein said diabetic complication is selected from the group consisting of diabetic neuropathy, nephropathy and retinopathy.

17. A composition as claimed in claim 16, wherein said compound is selected from the group consisting of small organic molecules, peptides, polypeptides, antisense molecules, oligonucleotides, polynucleotides, fatty acids and derivatives thereof.

18. The use of a composition as claimed in claim 14 for treating a lipid metabolic disorders.

19. The use of claim 18, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

20. The use of claim 19, wherein said diabetic complication is selected from the group consisting of diabetic neuropathy, nephropathy and retinopathy.

21. A method for diagnosing the presence of or a predisposition for a lipid metabolic disorder in a subject by detecting a germline alteration in a segment of claim 1 in said subject, comprising comparing the germine sequence of a segment of claim 1 from a tissue sample from said subject with the germline sequence of a wild-type of said segment, wherein an alteration in the germline sequence of said subject indicates the presence of or a predisposition to said lipid metabolic disorder.

22. A method for diagnosing the presence of or a predisposition for a disorder as claimed in claim 21, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

23. A method for diagnosing the presence of or a predisposition for a disorder as claimed in claim 22, wherein said diabetic complication is selected from the group consisting of diabetic neuropathy, nephropathy and retinopathy.

24. The method of claims 21 to 23, wherein said comparing is performed by a method selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

25. A method for identifying a compound which inhibits or promotes the activity of a polynucleotide, wherein the polynucleotide encodes a mammalian delta-6-desaturase, comprising the steps of:
(a) selecting a control animal having said polynucleotide and a test animal having said polynucleotide;
(b) treating said test animal using a compound; and,
(c) determining the relative quantity of an expression product of said polynucleotide, as between said control animal and said test animal.

26. A method of claim 25, wherein said animals are mammals.

27. A method of claim 26, wherein said mammals are rats.

28. A method for identifying a compound which inhibits or promotes the activity of a mammalian delta-6-desaturase, comprising the steps of:
(a) selecting a host cell of claim 4;
(b) cloning said host cell and separating said clones into a test group and a control group;
(c) treating said test group using a compound; and
(d) determining the relative quantity of an expression product of an expression polynucleotide operably linked to said polynucleotide segment, as between said test group and said control group.

29. A method for identifying a compound which inhibits or promotes the activity of a mammalian delta-6-desaturase, comprising the steps of:
(a) selecting a test group having a host cell of claim 4, a part thereof or an isolated polynucleotide thereof and a control group;
(b) treating said test group using a compound; and
(c) determining the relative quantity or relative activity of a product of said polynucleotide segment or of the said polynucleotide segment, as between said test group and said control group.

30. A method for identifying a compound which inhibits or promotes the activity of a mammalian delta-6-desaturase, comprising the steps of:
(a) selecting a control animal having a polypeptide segment of claim 1 and a test animal having said polypeptide segment;
(b) treating said test animal using a compound;
(c) determining the relative quantity or relative activity of an expression product of said polypeptide segment or of the said polypeptide segment, as between said control animal and said test animal.

31. A method of claim 30, wherein said animals are mammals.

32. A method of claim 31, wherein said mammals are rats.

33. A method for identifying a compound according to any one of the claims 25 to 32, wherein said relative activity of said expression product is determined by assaying for a conversion of 18:2n6 to 22:5n6.

34. A method for identifying a compound according to any one of the claims 25 to 32, wherein said relative activity of said expression product is determined by assaying for a conversion of 18:3n3 to 22:6n3.

35. A method for identifying a compound according to any one of the claims 25 to 32, wherein said relative activity of said expression product is determined by assaying for a conversion of 16:0 to 22:4n9.

36. A use of a method according to any one of the claims 25 to 34 for identifying a modulator that modulates lipid metabolism disorders.

37. A use according to claim 36 for identifying a modulator that modulates a disorder selected from the group consisting of atopic eczema, mastalgia, rheumatoid arthritis, Sjögren's syndrome, gastrointestinal disorders, viral infections and postviral fatigue, pre-menstral syndrome, endometriosis, cystic fibrosis, schizophrenia, alcoholism, congenital liver disease, Alzieimer's syndrome, Crohn's disease, cardiovascular disease, cancer, diabetes and diabetic complications.

38. A use according to claims 37, wherein said diabetic complication is selected from the group consisting of diabetic neuropathy, diabetic nephropathy and diabetic retinopathy.

39. A composition for treating a lipid metabolism disorder comprising a compound identified by any one of the methods of claims 25 to 35 and a pharmaceutically acceptable carrier.

40. A composition as claimed in claim 39, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

41. A composition as claimed in claim 40, wherein said diabetic complication is selected from the group consisting of diabetic neuropathy, nephropathy and retinopathy.

42. A composition as claimed in any one of claims 39 to 41, wherein said compound is selected from the group consisting of small organic molecules, peptides, polypeptides, antisense molecules, oligonucleotides, polynucleotides, fatty acids and derivatives thereof.

43. The use of a composition as claimed in claims 39 to 40 for treating lipid metabolism disorders.

44. The use of claim 43, wherein said disorder is selected from the group consisting of atopic eczema, mastalgia, rheumatoid arthritis, Sjögren's syndrome, gastrointestinal disorders, viral infections and postviral fatigue, premenstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

45. The use of claim 44, wherein said diabetic complication is selected from the group consisting of diabetic neuropathy, nephropathy and retinopathy.

46. A method for diagnosing the presence of or a predisposition for a lipid metabolism disorder in a subject by detecting a germline alteration in a polynucleotide of claim 1 in said subject, wherein the polynucleotide encodes a mammalian delta-6-desaturasecomprising comparing the germline sequence of said polynucleotide from a tissue sample from said subject with the germline sequence of a wild-type of said polynucleotide, wherein an alteration in the germline sequence of said subject indicates the presence of or a predisposition to said lipid metabolism disorder.

47. A method for diagnosing the presence of or a predisposition for a disorder as claimed in claim 46, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

48. A method for diagnosing the presence of or a predisposition for a disorder as claimed in claim 47, wherein said diabetic complication is selected from the group consisting of diabetic neuropathy, nephropathy and retinopathy.

49. The method of claims 46 to 48, wherein said comparing is performed by a method selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

50. A method for diagnosing the presence of or a predisposition for a lipid metabolic disorder in a subject, comprising comparing the polypeptide sequence of a control region of delta-6desaturase from a tissue sample from said subject with the sequence of a wild-type of said delta-6-desaturase, wherein an alteration in the sequence of said subject as compared to said wild-type indicates the presence of or a predisposition to said lipid metabolic disorder.

51. A method as claimed in claim 50, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

52. A method as claimed in any one of claims 50 to 51, wherein said diabetic complication is selected from the group consisting of diabetic neuropathy, nephropathy and retinopathy.

53. The method of any one of claims 50 to 52, wherein said comparing is performed by a method selected from the group consisting of immunoblotting, immunocytochemistry, enzyme-linked immunosorbent assay, DNA fingerprinting, radioimmunoassay, immunoradiometric assay, immunoenzymatic assay and polypeptide microarrays.

54. A method for identifying a compound which inhibits or promotes the activity of control regions of mammalian delta-6- and/or delta-5-desaturases, comprising the steps of:
(a) selecting one or more host cells comprising said polynucleotides, wherein such host cells are heterogeneous to said polynucleotides;
(b) cloning said host cells and separating said clones into a test group and a control group;
(c) treating said test group using a compound; and
(d) determining the relative quantities of expression products of operably linked polynucleotides to said control regions, as between said test group and said control group.

55. A composition for treating a lipid metabolism disorder comprising a compound identified by a method of claim 54 and a pharmaceutically acceptable carrier.

56. A composition as claimed in claim 55, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

57. A composition as claimed in claim 56, wherein said diabetic complication is selected from the group consisting of diabetic neuropathy, nephropathy and retinopathy.

58. A composition as claimed in any one of claims 55 to 57, wherein said compound is selected from the group consisting of small organic molecules, peptides, polypeptides, antisense molecules, oligonucleotides, polynucleotides, fatty acids and derivatives thereof.

59. The use of a composition as claimed in any one of claims 55 to 58 for treating lipid metabolism disorders.

60. The use of claim 59, wherein said disorder is selected from the group consisting of atopic eczema, mastalgia, rheumatoid arthritis, Sjögren's syndrome, gastrointestinal disorders, viral infections and postviral fatigue, premenstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

61. The use of claim 60, wherein said diabetic complication is selected from the group consisting of diabetic neuropathy, nephropathy and retinopathy.

62. A compound identified by the methods of any one of claims 9 to 13 or 25 to 35.

63. The use of a compound as claimed in claim 62 for treating a lipid metabolism disorder.

64. The use of claim 63, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

65. The use of claim 64, wherein said diabetic complication is selected from the group consisting of diabetic neuropathy, nephropathy and retinopathy.

66. A host cell as claimed in claim 4, wherein said host cell is a spheroplast

67. A spheroplast as claimed in claim 66, wherein said spheroplast is a Saccharomyces cerevisiae.

68. A method as claimed in any one of claims 5, 12, 13, 28, 29 or 54, wherein said host cell is a spheroplast.

69. A method as claimed in claim 68, wherein said spheroplast is a Saccharomyces cerevisiae.

70. A composition for treating a lipid metabolism disorder comprising a compound identified by any one of the methods of claims 68 or 69 and a pharmaceutically acceptable carrier.

71. A composition as claimed in claim 70, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

72. A composition as claimed in claim 71, wherein said diabetic complication is selected from the group consisting of diabetic neuropathy, nephropathy and retinopathy.

73. A composition as claimed in any one of claims 70 to 72, wherein said compound is selected from the group consisting of small organic molecules, peptides, polypeptides, antisense molecules, oligonucleotides, polynucleotides, fatty acids and derivatives thereof.

74. The use of a composition as claimed in any one of claims 70 to 73 for treating lipid metabolism disorders.

75. The use of claim 74, wherein said disorder is selected from the group consisting of atopic eczema, mastalgia, rheumatoid arthritis, Sjögren's syndrome, gastrointestinal disorders, viral infections and postviral fatigue, pre-menstrual syndrome, endometriosis, cystic fibrosis, schizophrenia, alcoholism, congenital liver disease, Alzheimer's syndrome, Crohn's disease, cardiovascular disease, cancer, diabetes and diabetic complications.

76. The use of claim 75, wherein said diabetic complication is selected from the group consisting of diabetic neuropathy, nephropathy and retinopathy.


SEQUENCE LISTING (70 sequences)

SEQ ID NO: 1 - DNA, 1595 bp, rat
  misc-feature 1558: n = a,c,t or g
ctgctgttcatccttctgagaactctcagaactttctcactaacgtcgtttgtgtattccggtgatccatctttagaaaacaaacaaaccaatacaaaaccgctttcttgactttattatgtcacatcgttaacatgtataaaagccagtcttgctccgtatgcatagctgtgtcccatctccctacaggatcttgctttgcagcccagaccagcctcaaactctggcaatattctgcctcaggcttctgattgttgacagtatggacatggccaccacacctacttcttatctttccggcgggcaggcggcactgtttggatgtttgtggcttcactcacattccttgggcctggagtgtgtggaatgaagaaaaggtgacggatgccaggacagaatgggatgaggatgccgtgggagggactgtggggaggccattacccccgcaccaggtacttcatttggaccagcatcatgaggatctagggagcccaaagtctggaaagggccgtggaagagaccaagcagggccactagctaagcatgtgcattttgaccattttatctctcttaacctcagactcttaattttgaagtgagtgataacaatattacagggttgtggtgagactttagtggacaggcccctcttgctactcccttagtatcagcatctttctttcctgtaattggtgcactattgtcgaatcaattgcgggcttttaatgcttccatgtttcccaaccaggcttgtgaactctttttcagtcaaagacaggaacggtgtccagttcatcctggcatcctccccaggagccacccccgcggcacttagcacgaggaagaagcagaggttctgcaattcttttccaagatggtctgataaaaggagtcagagccctggttaaagagaaacccacttctgactcctttctctaggaaggttgctggaaggcaacaaacaatatggatcgcttagacaatgcgagatacgggagtctgcgtcccagctagccagggagagaccgtcgcctagccaagacagcacaggggcccttgtccagccggaggccgagtgttggtcaccaaagccgttgcgcctcctttgtgcgagcatcaggtgcaaatccccggagcgcgggccgagcgcgctcgcgctttgaagcgcgcaaagaaaagcccagccgccgcccccgctctccctctcagctcctcagagatctactccgtctttctccgctccagccagcgaagggaggaggaggtgtcgagggccctgagctacccgggagttttcactagaggcaaaagtccacagcaggcgggccgagggcgggtggaggaaggggaccgctggggggcactgggaagccaggggtactcctgagggaaggcaggggtactggctagctagtgtaggcgaggaggagcgaaggcgaggggaggaggcgcggtgggaggaggagtagaagacaaaagctgaaagcgaggagggcgcctaccgcacactgcggccaggagagggaccgtcggtgctacgagccgcgggcgcccgganggccgtagcgcacgggtcatccgggtagacaggcagc

SEQ ID NO: 2 - DNA, 1497 bp, Human
tctcaggctctccattttcaagtgagatgtaataatatgcgtcctgtttacctctcagcctgtgatgagaatctaatgattagggtgtgctagcacacaggcacctgtaaatcccattgaaatctgagggccctataactcctctagtgattcccaagcctcgtgcaccccaccctctctgttcatccttacttcccacgtgtcccgttagccctccggatgcagtcaggcccatttcccccaggacgcccggcactaagccgccccatccagctggggtctgaggggcctgtctcttgccccacgcctaaaagacctaaccctcctcctatcccttcgatacggcagtctttatttgctggagtctgcacaacatcactgcccaatgatgtgtctgcttccacgattcccaaagagactgagcttactgagaccagggcaaggaccgcgccagttcctcatcgcccccttcctctgccttcccctcccttccccctcaccgcagccatggcgcccagaccaagaaagcagagcagaggttccgcaattcttttctaagattgtctgactagagggttcaaagccctctaatccaaggccggcttgtgtctctagggaggttgcagaaaggcgccagaatgtggatggcgcggacaatgtgggatactggagtctcgactgccgcgcgccaaggaagggcgtcacctagccaagccggccctggagttgagtgccccgcagcccgccggtccacccgcccggccgcttgctggcaccctggtggccgcgccggctttgtgtcggcatcaggtgcaaacccccagagcgccgggcgtgcgcgcacgcgccccggagcgcgcggagcccattcgttgcccccaccgggatctcctctaggatctcctccggcgcccggggccggagagtgggggagggaggaggtcggacacgtagcctgcctcgcagcagggctcgactccacgcgggagggcgggggagccggggacccgccgctccagcccgctggccttcgaaagatcctcctgggccaatggcaggcggggcgacgcgaccggattggtgcaggcgctctgctgatcgctgtggaaactcgggcggcggggaacgcgggaggatgtggaacccgaggcggggggagccggaggggcgggcagaggaggtgtcgaggccctgagctcccggggagtttttactggaggcaaaagtccatagcgggagggctgagggaggggcggaggaaggggaccgcttgggggcactgggaagccagggatcctccgccaggaaggcagggacactcccgagcgcaggcgagaaggctgggggagggggcgcggtgggaggagtaggagaagacaaaagccgaaagcgaagagggcccgggctgcacacaccggctgggaggcagccgtctgtgcagcgagcagccggcgcggggaggccgcagtgcacggggcgtcacagtcggcaggcagc

SEQ ID NO: 3 - DNA, 1335 bp, rat
atggggaagggaggtaaccagggagaggggagcaccgagctccaggctccgatgcccaccttccgctgggaggagattcagaagcacaacctgcgcaccgaccggtggctcgtcatcgaccgcaaggtctacaacgttaccaaatggtcccagcggcacccaggggggcaccgtgtcatcggacactattcgggagaagatgctacggatgccttccgtgccttccacctggacctggatttcgtgggcaagttcttgaagcccctgctgattggtgagctggccccagaggagcccagcctggaccggggcaaaagctctcagatcaccgaggacttcagggccctgaagaagactgctgaggacatgaaccttttcaaaaccaaccacctgttcttctttctcctcctgtcccacatcatcgtcatggaaagcatcgcctggttcatcctctcgtacttcggcaatggctggattcccaccgtcatcacggcctttgtccttgctacctcccaggcccaagctggatggctacaacatgattatggccacctttctgtctataagaaatccatatggaaccacattgtccacaagtttgtcattggccacttaaagggtgcctccgccaactggtggaaccatcgacatttccagcaccatgcgaagcccaacatcttccacaaggaccccgacataaagagcctgcacgtgtttgcccttggagagtggcagcccctcgagtatggcaagaagaagctgaaatatctgccctacaaccaccagcatgaatacttcttcctgatcggaccaccgctgctcatccctatgtacttccagtaccagatcatcatgaccatgatcagacgcagagactgggtggacttggcctgggccatcagctactatgcacgtttcttctacacctatatccctttctatggcatcttgggagccctggttttcctcaactttatcaggttcctggagagccactggtttgtgtgggtcacacagatgaaccacattgtcatggagattgatcttgatcactaccgggactggttcagcagccagctggcagccacctgcaatgtggagcagtccttcttcaatgactggttcagcgggcacctcaatttccagattgagcaccacctcttccccactatgccaagacacaacttgcacaagattgccccactggtgaagtctctctgcgccaagcatggcattgaataccaagagaagccgctgctgagggccctgctcgacattgtgagttcactgaagaagtctggggagctgtggctggatgcctacctccacaaatga

SEQ ID NO: 4 - DNA, 1335 bp, Human
atggggaagggagggaaccagggcgagggggccgccgagcgcgaggtgtcggtgcccaccttcagctgggaggagattcagaagcataacctgcgcaccgacaggtggctggtcattgaccgcaaggtttacaacatcaccaaatggtccatccagcacccggggggccagcgggtcatcgggcactacgctggagaagatgcaacggatgccttccgcgccttccaccctgacctggaattcgtgggcaagttcttgaaacccctgctgattggtgaactggccccggaggagcccagccaggaccacggcaagaactcaaagatcactgaggacttccgggccctgaggaagacggctgaggacatgaacctgttcaagaccaaccacgtgttcttcctcctcctcctggcccacatcatcgccctggagagcattgcatggttcactgtcttttactttggcaatggctggattcctaccctcatcacggcctttgtccttgctacctctcaggcccaagctggatggctgcaacatgattatggccacctgtctgtctacagaaaacccaagtggaaccaccttgtccacaaattcgtcattggccacttaaagggtgcctctgccaactggtggaatcatcgccacttccagcaccacgccaagcctaacatcttccacaaggatcccgatgtgaacatgctgcacgtgtttgttctgggcgaatggcagcccatcgagtacggcaagaagaagctgaaatacctgccctacaatcaccagcacgaatacttcttcctgattgggccgccgctgctcatccccatgtatttccagtaccagatcatcatgaccatgatcgtccataagaactgggtggacctggcctgggccgtcagctactacatccggttcttcatcacctacatccctttctacggcatcctgggagccctccttttcctcaacttcatcaggttcctggagagccactggtttgtgtgggtcacacagatgaatcacatcgtcatggagattgaccaggaggcctaccgtgactggttcagtagccagctgacagccacctgcaacgtggagcagtccttcttcaacgactggttcagtggacaccttaacttccagattgagcaccacctcttccccaccatgccccggcacaacttacacaagatcgccccgctggtgaagtctctatgtgccaagcatggcattgaataccaggagaagccgctactgagggccctgctggacatcatcaggtccctgaagaagtctgggaagctgtggctggacgcctaccttcacaaatga

SEQ ID NO: 5 - protein, 444 aa, rat
MGKGGNQGEGSTELQAPMPTFRWEEIQKHNLRTDRWLVIDRKVYNVTKWSQRHPGGHRVIGHYSGEDATDAFRAFHLDLDFVGKFLKPLLIGELAPEEPSLDRGKSSQITEDFRALKKTAEDMNLFKTNHLFFFLLLSHIIVMESIAWFILSYFGNGWIPTVITAFVLATSQAQAGWLQHDYGHLSVYKKSIWNHIVHKFVIGHLKGASANWWNHRHFQHHAKPNIFHKDPDIKSLHVFVLGEWQPLEYGKKKLKYLPYNHQHEYFFLIGPPLLIPMYFQYQIIMTMIRRRDWVDLAWAISYYARFFYTYIPFYGILGALVFLNFIRFLESHWFVWVTQMNHIVMEIDLDHYRDWFSSQLAATCNVEQSFFNDWFSGHLNFQIEHHLFPTMPRHNLHKIAPLVKSLCAKHGIEYQEKPLLRALLDIVSSLKKSGELWLDAYLHK

SEQ ID NO: 6 - protein, 473 aa, rat
MGKGGNQGEGSTELQAPMPTFRWEEIQKHNLRTDRWLVIDRKVYNVTKWSQRHPGGHRVIGHYSGEDATDAFRAFHLDLDFVGKFLKPLLIGELAPEEPSLDRGKSSQITEDFRALKKTAEDMNLFKTNHLFFFLLLSHIIVMESIAWFILSYFGNGWIPTVITAFVLATSQAQAGWLQHDYGHLSVYKKSIWNHIVHKFVIGHLKGASANWWNHRHFQHHAKPNIFHKDPDIKSLHVFALGEWQPLEYGKKKLKYLPYNHQHEYFFLIGPPLLIPMYFQYQIIMTMIRRRDWVDLAWAISYYARFFYTYIPFYGILGALVFLNFIRFLESHWFVWVTQMNHIVMEIDLDHYRDWFSSQLAATCNVEQSFFNDWFSGHLNFQIEHHLFPTMPRHNLHKIAPLVKSLCAKHGIEYQEKPLLRALLDIVSSLKKSGELWLDAYLHKSRGPFEGKPIPNPLLGLDSTRTGHHHHHH

SEQ ID NO: 7 - protein, 444 aa, Human
MGKGGNQGEGAAEREVSVPTFSWEEIQKHNLRTDRWLVIDRKVYNITKWSIQHPGGQRVIGHYAGEDATDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHHLFPTMPRHNLHKIAPLVKSLCAKHGIEYQEKPLLRALLDIIRSLKKSGKLWLDAYLHK

SEQ ID NO: 8 - protein, 473 aa, Human
MGKGGNQGEGAAEREVSVPTFSWEEIQKHNLRTDRWLVIDRKVYNITKWSIQHPGGQRVIGHYAGEDATDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHHLFPTMPRHNLHKIAPLVKSLCAKHGIEYQEKPLLRALLDIIRSLKKSGKLWLDAYLHKSRGPFEGKPIPNPLLGLDSTRTGHHHHHH

SEQ ID NO: 9 - DNA, 33 bp, Artificial Sequence
  Description of Artificial Sequenceprimer
cacgcgaagcttatggggaagggaggtaaccag

SEQ ID NO: 10 - DNA, 33 bp, Artificial Sequence
  Description of Artificial Sequenceprimer
cacgcgtctagatcatttgtggaggtaggcatc

SEQ ID NO: 11 - DNA, 33 bp, Artificial Sequence
  Description of Artificial Sequenceprimer
cacgcgtctagatttgtggaggtaggcatccag

SEQ ID NO: 12 - DNA, 30 bp, Artificial Sequence
  Description of Artificial Sequenceprimer
cacgcgaagcttatggggaagggagggaac

SEQ ID NO: 13 - DNA, 32 bp, Artificial Sequence
  Description of Artificial Sequenceprimer
cacgactctagaggggctgtggcttcatttgt

SEQ ID NO: 14 - DNA, 33 bp, Artificial Sequence
  Description of Artificial Sequenceprimer
cacgcgtctagatttgtgaaggtaggcgtccag

SEQ ID NO: 15 - DNA, 20 bp, Artificial Sequence
  Description of Artificial Sequenceprimer
tggggaagatcctctctgtg

SEQ ID NO: 16 - DNA, 20 bp, Artificial Sequence
  Description of Artificial Sequenceprimer
ggacttggcctggatgatta

SEQ ID NO: 17 - DNA, 20 bp, Artificial Sequence
  Description of Artificial Sequenceprimer
actattggggctgaaagcct

SEQ ID NO: 18 - DNA, 20 bp, Artificial Sequence
  Description of Artificial Sequenceprimer
catcgtgggaaaaagatggt

SEQ ID NO: 19 - DNA, 20 bp, Artificial Sequence
  Description of Artificial Sequenceprimer
gggaaaaagatggtgctcaa

SEQ ID NO: 20 - DNA, 20 bp, Artificial Sequence
  Description of Artificial Sequenceprimer
atgatcaatgtgcatgggaa

SEQ ID NO: 21 - DNA, 20 bp, Artificial Sequence
  Description of Artificial Sequenceprimer
ggcactacgctggagaagat

SEQ ID NO: 22 - DNA, 18 bp, Artificial Sequence
  Description of Artificial Sequenceprimer
ccattcgcccagaacaaa

SEQ ID NO: 23 - DNA, 20 bp, Artificial Sequence
  Description of Artificial Sequenceprimer
cccctgctgattggtgaact

SEQ ID NO: 24 - DNA, 20 bp, Artificial Sequence
  Description of Artificial Sequenceprimer
aaaggccgtgatgagggtag

SEQ ID NO: 25 - DNA, 20 bp, Artificial Sequence
  Description of Artificial Sequenceprimer
acacaaaccagtggctctcc

SEQ ID NO: 26 - DNA, 21 bp, Artificial Sequence
  Description of Artificial Sequenceprimer
ccatgatcgtccataagaact

SEQ ID NO: 27 - DNA, 22 bp, Artificial Sequence
  Description of Artificial Sequenceprimer
atttgtgaaggtaggcgtccag

SEQ ID NO: 28 - DNA, 21 bp, Artificial Sequence
  Description of Artificial Sequenceprimer
catccctttctacggcatcct

SEQ ID NO: 29 - DNA, 20 bp, Artificial Sequence
  Description of Artificial Sequenceprimer
gcggcttctcctggtattca

SEQ ID NO: 30 - DNA, 20 bp, Artificial Sequence
  Description of Artificial Sequenceprimer
gggccgtcagctactacatc

SEQ ID NO: 31 - DNA, 20 bp, Artificial Sequence
  Description of Artificial Sequenceprimer
tggctactgaaccagtcacg

SEQ ID NO: 32 - DNA, 21 bp, Artificial Sequence
  Description of Artificial Sequenceprimer
ccgaggacatgaagctgtttg

SEQ ID NO: 33 - DNA, 22 bp, Artificial Sequence
  Description of Artificial Sequenceprimer
ctgctggttgtagggtaggtat

SEQ ID NO: 34 - DNA, 21 bp, Artificial Sequence
  Description of Artificial Sequenceprimer
agtcccaccttctttgctttc

SEQ ID NO: 35 - DNA, 22 bp, Artificial Sequence
  Description of Artificial Sequenceprimer
agggtaggtatctgcgtttctt

SEQ ID NO: 36 - DNA, 22 bp, Artificial Sequence
  Description of Artificial Sequenceprimer
cttctttgttgctgtcagggtc

SEQ ID NO: 37 - DNA, 21 bp, Artificial Sequence
  Description of Artificial Sequenceprimer
ggtgctcgatctggaagttga

SEQ ID NO: 38 - DNA, 21 bp, Artificial Sequence
  Description of Artificial Sequenceprimer
aaaatctggcgtacatgctgg

SEQ ID NO: 39 - DNA, 20 bp, Artificial Sequence
  Description of Artificial Sequenceprimer
aggtggtgctcgatctggaa

SEQ ID NO: 40 - DNA, 21 bp, Artificial Sequence
  Description of Artificial Sequenceprimer
tcaacttccagatcgagcacc

SEQ ID NO: 41 - DNA, 20 bp, Artificial Sequence
  Description of Artificial Sequenceprimer
tccagccagatgtcaccaga

SEQ ID NO: 42 - DNA, 20 bp, Artificial Sequence
  Description of Artificial Sequenceprimer
gatgtggcccagtaggaaag

SEQ ID NO: 43 - DNA, 20 bp, Artificial Sequence
  Description of Artificial Sequenceprimer
gttcctacagcccctgttga

SEQ ID NO: 44 - DNA, 26 bp, Artificial Sequence
  Description of Artificial Sequenceprimer
ccacccacttctttcgctggataaca

SEQ ID NO: 45 - DNA, 24 bp, Artificial Sequence
  Description of Artificial Sequenceprimer
tgtgctggtggttgtacggcatat

SEQ ID NO: 46 - DNA, 33 bp, Artificial Sequence; identical to SEQ ID NO: 9
  Description of Artificial Sequenceprimer

SEQ ID NO: 47 - DNA, 33 bp, Artificial Sequence; identical to SEQ ID NO: 10
  Description of Artificial Sequenceprimer

SEQ ID NO: 48 - DNA, 33 bp, Artificial Sequence; identical to SEQ ID NO: 11
  Description of Artificial Sequenceprimer

SEQ ID NO: 49 - DNA, 30 bp, Artificial Sequence; identical to SEQ ID NO: 12
  Description of Artificial Sequenceprimer

SEQ ID NO: 50 - DNA, 32 bp, Artificial Sequence; identical to SEQ ID NO: 13
  Description of Artificial Sequenceprimer

SEQ ID NO: 51 - DNA, 33 bp, Artificial Sequence; identical to SEQ ID NO: 14
  Description of Artificial Sequenceprimer

SEQ ID NO: 52 - DNA, 22 bp, Artificial Sequence
  Description of Artificial Sequenceprimer
gtaatacgactcactatagggc

SEQ ID NO: 53 - DNA, 24 bp, Artificial Sequence
  Description of Artificial Sequenceprimer
caggttgtgcttctgaatctcctc

SEQ ID NO: 54 - DNA, 19 bp, Artificial Sequence
  Description of Artificial Sequenceprimer
actatagggcacgcgtggt

SEQ ID NO: 55 - DNA, 27 bp, Artificial Sequence
  Description of Artificial Sequenceprimer
tctccctggttacctcccttccccatg

SEQ ID NO: 56 - DNA, 34 bp, Artificial Sequence
  Description of Artificial Sequenceprimer
cacgacgagctcctgctgttcattccttctgaga

SEQ ID NO: 57 - DNA, 33 bp, Artificial Sequence
  Description of Artificial Sequenceprimer
cacgacctcgaggctgcctgtctacccggatga

SEQ ID NO: 58 - DNA, 20 bp, Artificial Sequence
  Description of Artificial Sequenceprimer
tctcaggctctccattttca

SEQ ID NO: 59 - DNA, 20 bp, Artificial Sequence
  Description of Artificial Sequenceprimer
ctcttcgctttcggcttttg

SEQ ID NO: 60 - DNA, 30 bp, Artificial Sequence
  Description of Artificial Sequenceprimer
cacgcgctgcaggctgcctgccgactgtga

SEQ ID NO: 61 - DNA, 33 bp, Artificial Sequence
  Description of Artificial Sequenceprimer
gcgggtacctctcaggctctccattttcaagtg

SEQ ID NO: 62 - DNA, 27 bp, Artificial Sequence
  Description of Artificial Sequenceprimer
gcgggtaccgctgcctgccgactgtga

SEQ ID NO: 63 - DNA, 34 bp, Artificial Sequence; identical to SEQ ID NO: 56
  Description of Artificial Sequenceprimer

SEQ ID NO: 64 - DNA, 33 bp, Artificial Sequence; identical to SEQ ID NO: 57
  Description of Artificial Sequenceprimer

SEQ ID NO: 65 - DNA, 20 bp, Artificial Sequence
  Description of Artificial Sequenceprimer
tctcaggctctccattttca

SEQ ID NO: 66 - DNA, 20 bp, Artificial Sequence
  Description of Artificial Sequenceprimer
ctcttcgctttcggcttttg

SEQ ID NO: 67 - DNA, 20 bp, Artificial Sequence
  Description of Artificial Sequenceprimer
tctcaggctctccattttca

SEQ ID NO: 68 - DNA, 30 bp, Artificial Sequence; identical to SEQ ID NO: 60
  Description of Artificial Sequenceprimer

SEQ ID NO: 69 - DNA, 33 bp, Artificial Sequence; identical to SEQ ID NO: 61
  Description of Artificial Sequenceprimer

SEQ ID NO: 70 - DNA, 27 bp, Artificial Sequence
  Description of Artificial Sequenceprimer
gcgggtaccgctgcctgccgactgtga